(19) United States
(12) Patent Application Publication
(10) Pub. No.: US 2006/0183759 A1
(43) Pub. Date: Aug. 17, 2006

(54) Tissue transglutaminase inhibitors

(76) Inventors: Ross L. Stein, Sudbury, MA (US); April Case, Watertown, MA (US); Li-An Yeh, Cary, NC (US); Gregory D. Cuny, Somerville, MA (US); Eric Duval, Den Haag (NL)

Correspondence Address: WOLF GREENFIELD & SACKS, PC, FEDERAL RESERVE PLAZA, 600 ATLANTIC AVENUE, BOSTON, MA, 02210-2206, US

(21) Appl. No.: 11/294,674

(22) Filed: Dec. 5, 2005

Related U.S. Application Data
(60) Provisional application No. 60/633,400, filed on Dec. 3, 2004.

(51) Int. Cl. A61K 31/519; C07D 487/02; C07D 491/02; C07D 498/02

(52) U.S. Cl. 514/260.1; 544/278; 544/280; 514/265.1

(57) ABSTRACT

The present invention provides novel compounds and methods useful for treating transglutaminase associated disorders such as celiac spru, Alzheimer's disease and Huntington's disease. Certain compounds of the invention are tissue transglutaminase inhibitors that comprise thiophene moieties. Methods of the invention include treatment of transglutaminase associated disorders with inhibitors of transglutaminase.

### RELATED APPLICATIONS

This application claims benefit under 35 U.S.C. 119(e) of the filing date of U.S. Ser. No. 60/633,400 filed on Dec. 3, 2004, the entire disclosure of which is incorporated herein by reference.

### FIELD OF INVENTION

The invention relates to pharmaceutical compositions and methods for the treatment of transglutaminase associated disorders such as celiac spru, Alzheimer's disease and Huntington's disease with novel inhibitors of transglutaminase.

### BACKGROUND OF INVENTION

Transglutaminases are a family of Ca⁺²-dependent enzymes that catalyze the formation of isopeptide bonds between the carboxamide group of protein/peptide-bound glutamine residues and the ε-amino group of protein/peptide-bound lysine residues to form N^{ε}-(γ-L-glutamyl)-L-lysine cross links with loss of ammonia. Currently, eight transglutaminase isoforms have been identified. Transglutaminases are normally expressed at low levels in many different tissues and serve important roles, such as in blood clotting and epithelia formation. However, transglutaminase isozymes also are involved in diverse pathological conditions, such as celiac disease, inclusion body myositis, cataract formation, atherosclerosis and neurodegenerative disorders (Kim et al., Neurochem. Int. 2002, 40, 85-103; Gentile et al., Curr. Drug Targets CNS Neurol. Disord. 2004, 3(2), 99-104).

Tissue transglutaminases are involved in several general biological functions, including apoptosis, cell adhesion and signal transduction (Gentile et al., Curr. Drug Targets CNS Neurol. Disord. 2004, 3(2), 99-104). In addition, tissue transglutaminases have been linked to celiac disease (Dieterich et al., Nature Med. 30 1997, 3, 797-801), Alzheimer's disease (Grierson et al., Neurosci. Lett. 2001, 298, 9-12) and Huntington's disease (Mastroberardino et al., Cell. Death Diff. 2002, 9, 873-880). Therefore, potent and selective tissue transglutaminase inhibitors are needed in order to provide compounds and methods for therapeutic use.

### SUMMARY OF INVENTION

The present invention provides compounds and pharmaceutical preparations that are useful for treating disorders associated with tissue transglutaminase.

According to one aspect of the invention, the compound has the formula:

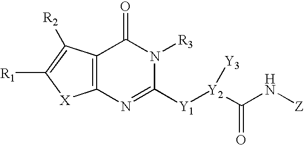

wherein R₁ is selected from the group consisting of: H, halogen, alkyl, substituted alkyl, aryl and substituted aryl; R₂ is selected from the group consisting of: H, alkyl, substituted alkyl, aryl and substituted aryl; R₃ is selected from the group consisting of: alkyl, substituted alkyl, aryl, substituted aryl, pyridine, substituted pyridine; X is selected from the group consisting of: S, O and NH; Y₁ is selected from the group consisting of: S, CH₂, NH, O, N-alkyl; Y₂ is selected from the group consisting of: CH, alkyl and substituted alkyl; Y₃ is selected from the group consisting of: H and CH₃; Z is selected from the group consisting of: OH and NH₂; with the provision that when X is S, Y₁ is S, Y₂ is CH, Y₃ is H, Z is NH₂, R₁ is Me, and R₂ is Me, R₃ can not be Ph or a propylene group (CH₂═CH—CH₂—); and with the provision that when X is S, Y₁ is S, Y₂ is CH, Y₃is H, Z is NH₂, R₁ is H, and R₂ is Ph, R₃ can not be Ph. In certain embodiments R₁ is selected from the group consisting of: H, Me and Cl; R₂ is selected from the group consisting of: phenyl and substituted phenyl; R₃ is selected from the group consisting of: phenyl and substituted phenyl; X is S; Y₁ is S; Y₂ is CH; Y₃ is H; and Z is NH₂.

According to one aspect of the invention, the compound has the formula:

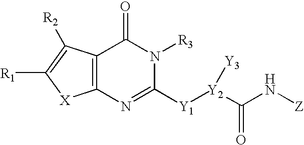

wherein R₁ is selected from the group consisting of: H, halogen, alkyl, substituted alkyl, aryl and substituted aryl; R₂ is selected from the group consisting of: H, alkyl, substituted alkyl, aryl and substituted aryl; R₃ is selected from the group consisting of: H, alkyl, substituted alkyl, aryl, substituted aryl, pyridine, substituted pyridine; X is selected from the group consisting of: S, O and NH; Y₁ is selected from the group consisting of: S, CH₂, NH, O, N-alkyl; Y₂ is selected from the group consisting of: CH, alkyl and substituted alkyl; Y₃ is selected from the group consisting of: H and CH₃; Z is selected from the group consisting of: OH and NH₂; with the provision that when X is S, Y₁ is S, Y₂ is CH, Y₃ is H, Z is NH₂, R₁ is Me, and R₂ is Me, R₃ can not be H.

According to another aspect of the invention, a method of treating a transglutaminase-associated disorder is provided comprising administering to a subject having the transglutaminase-associated disorder a compound of the formula:

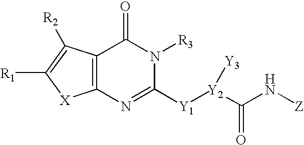

R₁ is selected from the group consisting of: H, halogen, alkyl, substituted alkyl, aryl and substituted aryl; R₂ is selected from the group consisting of: H, alkyl, substituted alkyl, aryl and substituted aryl; R₃ is selected from the group consisting of: H, alkyl, substituted alkyl, aryl, substituted aryl, pyridine, and substituted pyridine; X is selected from the group consisting of: S, O and NH; Y₁ is selected from the group consisting of: S, CH₂, NH, O and N-alkyl; Y₂ is selected from the group consisting of: CH, alkyl and substituted alkyl; Y₃ is selected from the group consisting of: H and CH₃; Z is selected from the group consisting of: OH and NH₂; and wherein the compound is administered in an amount effective to treat the transglutaminase-associated disorder.

According to another aspect of the invention, the compound has the formula:

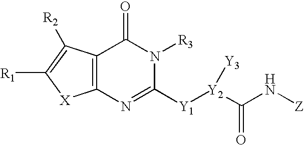

wherein R₁ is selected from the group consisting of: H, alkyl, and substituted alkyl; R₂ is selected from the group consisting of: H, alkyl, substituted alkyl, aryl and substituted aryl, ring system, substituted ring system, carbocyclic ring system, substituted carbocyclic ring system, heterocyclic ring system, and substituted heterocyclic ring system; R₃ is selected from the group consisting of: H, alkyl, substituted alkyl, aryl and substituted aryl, ring system, substituted ring system, carbocyclic ring system, substituted carbocyclic ring system, heterocyclic ring system, and substituted heterocyclic ring system; X is selected from the group consisting of: S, O and NH; Y₁ is selected from the group consisting of: S, CH₂, NH and N-alkyl; Y₂ is selected from the group consisting of: CH, alkyl and substituted alkyl; Y₃ is selected from the group consisting of: H and CH₃; Z is selected from the group consisting of: OH and NH₂; with the provision that when X is S, Y₁ is S, Y₂ is CH, Y₃ is H, Z is NH₂, R₁ is Me, and R₂ is Me, R₃ can not be Ph or a propylene group (CH₂═CH—CH₂—); and with the provision that when X is S, Y₁ is S, Y₂ is CH, Y₃ is H, Z is NH₂, R₁ is H, and R₂ is Ph, R₃ can not be Ph. In another aspect, Y₁ in this compound may be O.

According to another aspect of the invention, a method of treating a transglutaminase-associated disorder is provided comprising administering to a subject having the tissue transglutaminase disorder a compound of the formula:

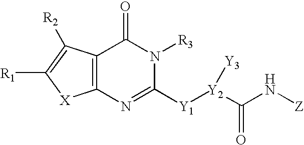

wherein R₁ is selected from the group consisting of: H, alkyl, and substituted alkyl; R₂ is selected from the group consisting of: H, alkyl, substituted alkyl, aryl and substituted aryl, ring system, substituted ring system, carbocyclic ring system, substituted carbocyclic ring system, heterocyclic ring system, and substituted heterocyclic ring system; R₃ is selected from the group consisting of: H, alkyl, substituted alkyl, aryl and substituted aryl, ring system, substituted ring system, carbocyclic ring system, substituted carbocyclic ring system, heterocyclic ring system, and substituted heterocyclic ring system, X is selected from the group consisting of: S, O and NH; Y₁ is selected from the group consisting of: S, CH₂, NH and N-alkyl; Y₂ is selected from the group consisting of: CH, alkyl and substituted alkyl; Y₃ is selected from the group consisting of: H and CH₃; Z is selected from the group consisting of: OH and NH₂; and wherein the compound is administered in an amount effective to treat the transglutaminase-associated disorder. In another aspect, Y₁ in this compound may be O.

According to another aspect of the invention, the compound has the formula:

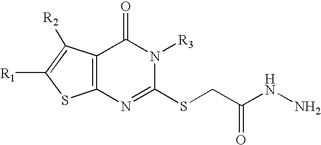

wherein R₁ is selected from the group consisting of: H, Cl, Me, iPr, Ph and substituted Ph; R₂ is selected from the group consisting of: Ph, substituted Ph and H; and R₃ is selected from the group consisting of: Ph and substituted Ph; with the provision that R₃ can not be Ph or a propylene group (CH₂═CH—CH₂—) when R₁ and R₂ are Me; and R₃ can not be Ph when R₁ is H and R₂ is Ph. In one embodiment, R₁ is selected from the group consisting of: H, Cl, Me, iPr, and Ph; R₂ is selected from the group consisting of: 4-F-Ph, Ph, Me, iPr, 4-OMe-Ph, 3-OMe-Ph, 2-OMe-Ph, 2-OH-Ph, 2-(OC₃H₆NEt₂)-Ph, 3-F-Ph, 2-F-Ph, 4-Cy-Ph, 2-CN-Ph, 3-CN-Ph, 4-CN-Ph, nitrile, 3-CF₃-Ph, 3-CF₃—O-Ph and H; and R₃ is selected from the group consisting of: 4-F-Ph, Me, CH₂Ph, 3-Py, Cy, 2-OMe-Ph, 3-OMe-Ph, 4-OMe-Ph, 2-Cl-Ph, 3-Cl-Ph, 4-Cl-Ph, 2-F-Ph, 3-F-Ph, 4-F-Ph, 2,5-di-F-Ph, 3,5-di-F-Ph, 2,6-di-F-Ph, 4-Cy-Ph, 2-CN-Ph, 3-CN-Ph, 4-CN-Ph, 3-CF₃-Ph, 3-CF₃—O-Ph and Ph; with the provision that R₃ can not be Ph or a propylene group (CH₂═CH—CH₂—) when R₁ and R₂ are Me; and with the provision that R₃ can not be Ph when R₁ is H and R₂ is Ph. In another embodiment, R₁ is selected from the group consisting of: H, Cl or Me; R₂ is selected from the group consisting of: Ph, 3-OMe-Ph, 2-OMe-Ph, and 2-F-Ph; and R₃ is 2-F-Ph or 3-F-Ph.

According to another aspect of the invention, a method of treating a transglutaminase-associated disorder is provided comprising administering to a subject having transglutaminase-associated disorder a compound of the formula:

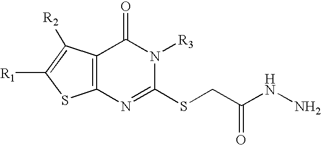

wherein R₁ is selected from the group consisting of: H, Cl, Me, iPr, Ph and substituted Ph; R₂ is selected from the group consisting of: Ph, substituted Ph and H; R₃ is selected from the group consisting of: Ph and substituted Ph; and wherein the compound is administered in an amount effective to treat the transglutaminase-associated disorder. In one embodiment, R₁ is selected from the group consisting of: H, Cl, Me, iPr, and Ph; R₂ is selected from the group consisting of: 4-F-Ph, Ph, Me, iPr, 4-OMe-Ph, 3-OMe-Ph, 2-OMe-Ph, 2-OH-Ph, 2-(OC₃H₆NEt₂)-Ph, 3-F-Ph, 2-F-Ph, 4-Cy-Ph, 2-CN-Ph, 3-CN-Ph, 4-CN-Ph, nitrile, 3-CF₃-Ph, 3-CF₃—O-Ph and H; and R₃ is selected from the group consisting of: 4-F-Ph, Me, CH₂Ph, 3-Py, Cy, 2OMe-Ph, 3-OMe-Ph, 4-OMe-Ph, 2-Cl-Ph, 3-Cl-Ph, 4-Cl-Ph, 2-F-Ph, 3-F-Ph, 4-F-Ph, 2,5-di-F-Ph, 3,5-di-F-Ph, 2,6-di-F-Ph, 4-Cy-Ph, 2-CN-Ph, 3-CN-Ph, 4-CN-Ph, 3-CF₃-Ph, 3-CF₃—O-Ph and Ph; with the provision that R₃ can not be Ph or a propylene group (CH₂═CH—CH₂—) when R₁ and R₂ are Me; and with the provision that R₃ can not be Ph when R₁ is H and R₂ is Ph. In yet another embodiment, R₁ is selected from the group consisting of: H, Cl or Me; R₂ is selected from the group consisting of: Ph, 3-OMe-Ph, 2-OMe-Ph, and 2-F-Ph; and R₃ is 2-F-Ph or 3-F-Ph.

According to another aspect of the invention, the compound has the formula:

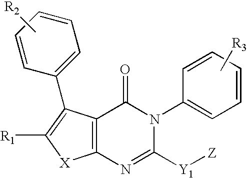

wherein R₁ is selected from the group consisting of: H, halogen and Me; R₂ is selected from the group consisting of: H, 4-F, and 2-F; R₃ is selected from the group consisting of: H, 4-F, and 3-F; X is selected from the group consisting of: S, O and NH; Y₁ is selected from the group consisting of: S, O, NH and NMe; Z is selected from the group consisting of: CH₂C(O)NHNH₂, CH₂CH₂C(O)NHNH₂, CH(Me)C(O)NHNH₂, CH₂C(O)NMeNH₂, CH₂C(O)NHNHMe, CH₂CO₂H, CH₂CO₂Et, CH₂C(O)NHMe, CH₂C(O)NH₂, CH₂C(O)NHOH, and CH₂C(O)CH₂NH₂. In one embodiment, R₁ is Cl or F.

According to another aspect of the invention, a method of treating a transglutaminase-associated disorder is provided comprising administering to a subject having the transglutaminase-associated disorder a compound of the formula:

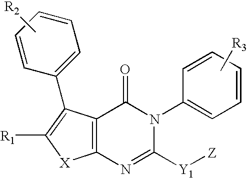

wherein R₁ is selected from the group consisting of: H, halogen and Me; R₂ is selected from the group consisting of: H, 4-F, and 2-F; R₃ is selected from the group consisting of: H, 4-F, and 3-F; X is selected from the group consisting of: S, O and NH; Y₁ is selected from the group consisting of: S, O, NH and NMe; Z is selected from the group consisting of: CH₂C(O)NHNH₂, CH₂CH₂C(O)NHNH₂, CH(Me)C(O)NHNH₂, CH₂C(O)NMeNH₂, CH₂C(O)NHNHMe, CH₂CO₂H, CH₂CO₂Et, CH₂C(O)NHMe, CH₂C(O)NH₂, CH₂C(O)NHOH, and CH₂C(O)CH₂NH₂; and wherein the compound is administered in an amount effective to treat the transglutaminase-associated disorder. In one embodiment, R₁ is Cl or F.

According to another aspect of the invention, the compound has the formula:

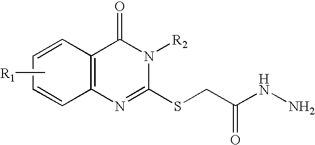

wherein R₁ is selected from the group consisting of: H, and Ph; and R₂ is Ph or 3-F-Ph.

According to another aspect of the invention, a method of treating a transglutaminase-associated disorder is provided comprising administering to a subject having the transglutaminase-associated disorder a compound of the formula:

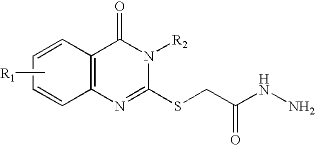

wherein R₁ is selected from the group consisting of: H, and Ph; and R₂ is Ph or 3-F-Ph; and wherein the compound is administered in an amount effective to treat the transglutaminase-associated disorder.

According to another aspect of the invention, the compound has the formula:

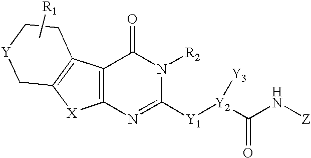

wherein Y is selected from the group consisting of: CH₂, N-Boc, NH, NMe and N-alkyl; and R₁ is selected from the group consisting of: H, Me, Ph, alkyl, arylalkyl, t-butyl, and CH₂Ph; R₂ is selected from the group consisting of: alkyl, substituted alkyl, aryl, substituted aryl, pyridine, and substituted pyridine; X is selected from the group consisting of: S, O and NH; Y₁ is selected from the group consisting of: S, CH₂, O, NH, N-alkyl; Y₂ is selected from the group consisting of: CH, alkyl and substituted alkyl; Y₃ is selected from the group consisting of: H and CH₃; and Z is selected from the group consisting of: OH and NH₂.

According to another aspect of the invention, a method of treating a transglutaminase-associated disorder is provided comprising administering to a subject having the transglutaminase-associated disorder a compound of the formula:

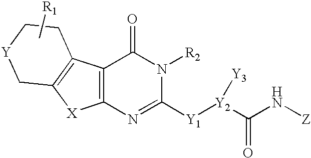

wherein Y is selected from the group consisting of: CH₂, N-Boc, NH, NMe and N-alkyl; and R₁ is selected from the group consisting of: H, Me, Ph, alkyl, arylalkyl, t-butyl, and CH₂Ph; R₂ is selected from the group consisting of: alkyl, substituted alkyl, aryl, substituted aryl, pyridine, and substituted pyridine; X is selected from the group consisting of: S, O and NH; Y₁ is selected from the group consisting of: S, CH₂, O, NH, alkyl; Y₂ is selected from the group consisting of: CH, alkyl and substituted alkyl; Y₃ is selected from the group consisting of: H and CH₃; Z is selected from the group consisting of: OH and NH₂ and wherein the compound is administered in an amount effective to treat the transglutaminase-associated disorder.

According to one aspect of the invention, one or more of R₁, R₂ and R₃, in any of the structures disclosed herein may be independently an aromatic ring or an electron deficient aromatic ring. Accordingly, in some compounds R₁ and R₂, or R₁ and R₃, or R₂ and R₃ may be electron deficient aromatic rings. In other compounds, each of R₁, R₂ and R₃ may be an electron deficient aromatic ring. In other compounds, only one of R₁, R₂ and R₃, is an electron deficient aromatic ring, etc. When any one or more of R₁, R₂ and R₃ are electron deficient aromatic rings, the remaining R₁, R₂ and/or R₃ groups may be an aromatic ring or another group as described herein.

According to another aspect of the invention, compounds of the invention are provided in a stereoisomeric form, or a pharmaceutically acceptable acid or base addition salt of the compound described herein.

Another aspect of the invention provides a pharmaceutical preparation comprising one or more compounds of the invention and a pharmaceutically acceptable carrier. In some embodiments, the pharmaceutically acceptable carrier is chosen from a diluent, a solid filler, and a solvent encapsulating material. In certain embodiments of the invention, the pharmaceutically acceptable carrier is chosen from a sugar, a starch, cellulose, powdered tragacanth, malt, gelatin, talc, an excipient, an oil, a glycol, a polyol, an ester, an agar, a buffering agent, alginic acid, pyrogen free water, isotonic saline, Ringer's solution, ethyl alcohol, a pH buffered solution, a polyester, a polyanhydride, and a polycarbonate.

In one aspect of the invention, the compounds of the invention are administered via a route chosen from oral, parenteral, topical, ocular, transdermal, and nasal routes. In certain aspects of the invention, the compounds of the invention are administered by subcutaneous, intramuscular, intravenous, or epidural injection. In certain embodiments, the compounds of the invention are administered in combination with a pharmaceutically acceptable carrier. In yet other embodiments, the compounds of the invention are administered in combination with another compound.

Another aspect of the invention provides a method of treating a transglutaminase-associated disorder (e.g. a tissue transglutaminase or transglutaminase-2 associated disorder) comprising administering to a subject having the transglutaminase-associated disorder one or more compounds of the invention in an amount effective to treat the transglutaminase-associated disorder.

In certain embodiments, the transglutaminase-associated disorder is a neurodegenerative disorder chosen from the list consisting of: Parkinson's disease, Alzheimer's disease, and progressive supranuclear palsy. In one embodiment, the transglutaminase-associated neurodegenerative disorder is Parkinson's disease or Alzheimer's disease.

In yet another embodiment, the transglutaminase-associated disorder is a CAG-expansion disorder chosen from the list consisting of: spinocerebellar ataxia type 1, spinocerebellar ataxia type 2, Machado-Joseph disorder, spinocerebellar ataxia type 6, spinocerebellar ataxia type 7, spinocerebellar ataxia type 12, spinocerebellar ataxia type 17, spinobulbar muscular atrophy, dentatorubral-pallidoluysian atrophy and Huntington's disease. In one embodiment, the transglutaminase-associated CAG-expansion disorder is Huntington's disease.

In another embodiment, the transglutaminase-associated disorder is an autoimmune disorder chosen from the list consisting of: hepatitis, hemolytic anemia, myasthenia, subepidermal blisters, multiple sclerosis, lupus, necrobiosis lipoidica, myasthenia gravis, bullous pemphigoid, Goodpasture disease, rheumatoid arthritis, amyloid lateral sclerosis, inclusion body myositis and celiac spru. In one embodiment, the transglutaminase-associated autoimmune disorder is celiac spru.

In yet another embodiment, the transglutaminase-associated disorder is selected from the list consisting of: inflammation, cataract forrnation and atherosclerosis.

In one aspect of the invention, any one or more of the compounds described herein may be used to inhibit one or more transglutaminases, including a tissue transglutaminase or transglutaminase-2, or to treat one or more transglutaminase associated disorders, including one or more tissue transglutaminase or transglutaminase-2 associated disorders.

An additional aspect of the invention relates to processes for producing one or more compounds comprising a thiophene, thienopyrimidinone, thienopyrimidinone acylhydrazide, or quinazolinone moiety.

In one aspect, the invention provides one or more intermediates that are useful to synthesize one or more transglutaminase inhibitors. In another aspect, the invention provides one or more methods for synthesizing one or more transglutaminase inhibitors or one or more intermediates in the synthesis of a transglutaminase inhibitor.

A further aspect of the present invention relates to the synthesis of combinatorial libraries of the heterocyclic compounds comprising a thiophene, thienopyrimidinone, thienopyrimidinone acylhydrazide, or quinazolinone moiety and the screening of those libraries for biological activity, e.g. in assays based on transglutaminase activity and in animal models of disease associated with tissue transglutaminase, celiac spru, Parkinson's disease, Alzheimer's disease, Huntington's disease etc.

In one aspect, one or more transglutaminase inhibitors of the invention may be administered to a subject (e.g. a patient) in combination with one or more other compounds (e.g. therapeutic agents). Two or more inhibitors and/or other compounds may be administered in combination in the form of a single composition that combines the inhibitors and/or other compounds. Alternatively, two or more inhibitors and/or other compounds may be administered in combination when they are provided as separate compositions that are nonetheless administered simultaneously, sequentially, or otherwise combined as part of a therapeutic regimen.

In one aspect, one or more compositions or combinations of the invention may be provided in a sterilized form (e.g. solution, suspension, gel, powder, or other solid, etc.). Compositions or combinations may be sterilized using any appropriate technique including filtration, heat sterilization, irradiation, chemical treatment, etc.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 depicts a general scheme for synthesizing a 2-hydroxy- and 2-thiohydroxy-3-H-thieno[2,3-d]pyrimidin-4-ones from aldehydes and ketones.

FIG. 2 depicts a scheme for synthesizing a compound comprising a thiophene moiety.

FIG. 3 depicts a scheme for synthesizing oxygen and nitrogen analogs of the compound comprising a thiophene moiety.

FIG. 4 depicts a general scheme for synthesizing an analog of a thiophene compound wherein the thiophene moiety has been replaced by benzene.

FIG. 5 depicts a scheme for the preparation thiophene derivatives.

FIG. 6 shows a scheme for the preparation of a phenol derivative of the thiophene compounds.

### DETAILED DESCRIPTION

In one aspect, the invention relates to compounds that may inhibit one or more transglutaminase activities, including one or more tissue transglutaminase or transglutaminase-2 activities. Transglutaminase (Tgase) activities include but are not limited to the formation of isopeptide bonds between the carboxamide group of protein/peptide-bound glutamine residues and the ε-amino group of protein/peptide-bound lysine residues to form N^{ε}-(γ-L-glutamyl)-L-lysine, cross links with loss of ammonia. Furthermore, transglutaminase activity is functionally related to cellular GTP and/or Ca²⁺ levels (Zhang et al., 1998; Andersson et al., 1998). Currently, eight TGase isoforms have been identified. TGases are normally expressed at low levels in many different tissues and serve vital roles, including but not limited to blood clotting, cellular envelope formation, epidermal differentiation, apoptosis, cell adhesion, signal transduction, and hair follicle differentiation. In one embodiment, inhibition of a transglutaminase activity or a transglutaminase-associated symptom may result in essentially a complete loss of that activity or symptom. In another embodiment, inhibition of a transglutaminase activity or transglutaminase-associated symptom may result in a partial reduction or decrease or loss of that activity or symptom (e.g. more than about a 5%, 10%, 25%, 50%, or 75% inhibition, or more or less inhibition).

One or more transglutaminase inhibitors may be used to treat a subject for which a decreased level of transglutaminase activity is beneficial. A decreased level of transglutaminase activity may be beneficial for a subject with aberrant transglutaminase activity. Aberrant transglutaminase activity may be associated with above-normal levels of transglutaminase expression; above-normal levels of transglutaminase activity due to the presence of one or more active transglutaminase variants such as a mutant form or an allelic variant; above-normal levels of transglutaminase activity due to other factors such as Ca²⁺, GTP, or other regulatory factors or proteins; abnormal transglutaminase activity including abnormal substrate selection; or any combination of the above. A decreased level of transglutaminase activity also may be beneficial for a subject with normal transglutaminases, if the subject has one or more other metabolic disorders or disturbances that result in abnormal isopeptide bond formation (or abnormally high levels of isopeptide bond formation) on at least one substrate.

In one aspect, embodiments of the invention may be useful to treat a subject suffering from a transglutaminase related disorder, including a tissue transglutaminase related disorder such as a celiac disease (e.g. celiac spru), Parkinson's disease, Alzheimer's disease, Huntington's disease, or other autoimmune or neurodegenerative diseases, and the like. One or more compounds of the invention also may be useful for understanding the patho-physiology of transglutaminase related diseases.

In a celiac disease (e.g. celiac spru), transglutaminase modification of a dietary peptide can result in the formation of products (e.g. peptide complexes) that are inflammatory or immunostimulatory. Accordingly, in one aspect, methods of the invention include preventing the formation of inflammatory or immunostimulatory products, particularly in a subject with a gluten-rich diet. In another aspect, methods of the invention include inhibiting transglutaminase activity on one or more dietary peptides. In one embodiment, methods of the invention include inhibiting the activity of tissue transglutaminase or transglutaminase-2 in the presence of one or more gluten peptides. In one embodiment, methods of the invention include inhibiting the modification of one or more gluten peptides. In one embodiment, methods of the invention include inhibiting the cross-linking of tissue transglutaminase or transglutaminase-2 onto one or more gluten peptides. In one embodiment, methods of the invention include inhibiting the cross-linking of tissue transglutaminase or transglutaminase-2 onto gliadin.

In another aspect, the invention relates to methods and compositions that are useful for synthesizing one or more transglutaminase inhibitors. A composition may be useful as an intermediate in the synthesis of a transglutaminase inhibitor, even if the composition has little or no transglutaminase inhibitory properties. Accordingly, in one aspect the invention relates to methods for synthesizing one or more intermediates described herein.

In another aspect, the invention relates to combinations comprising one or more transglutaminase inhibitors along with one or more additional therapeutic agents. Additional therapeutic agents may be other forms of transglutaminase inhibitors. Additional therapeutic agents may be agents that do not inhibit transglutaminase activity.

In one aspect of the invention, compounds may be low molecular weight molecules of the formula:

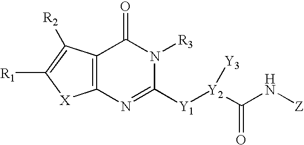

wherein R₁ is selected from the group consisting of: H, halogen, alkyl, substituted alkyl, aryl and substituted aryl; R₂ is selected from the group consisting of: H, alkyl, substituted alkyl, aryl and substituted aryl; R₃ is selected from the group consisting of: alkyl, substituted alkyl, aryl, substituted aryl, pyridine and substituted pyridine; X is selected from the group consisting of: S, O and NH; Y₁ is selected from the group consisting of: S, CH₂, NH, O and N-alkyl; Y₂ is selected from the group consisting of: CH, alkyl and substituted alkyl; Y₃ is selected from the group consisting of: H and CH₃; Z is selected from the group consisting of: OH and NH₂; with the provision that when X is S, Y₁ is S, Y₂ is CH, Y₃ is H, Z is NH₂, R1 is Me, and R2 is Me, R3 can not be Ph or a propylene group (CH₂═CH—CH₂—); and with the provision that when X is S, Y₁ is S, Y₂ is CH, Y₃is H, Z is NH₂, R₁ is H, and R₂is Ph, R₃ can not be Ph. In certain embodiments R₁ is selected from the group consisting of: H, Me and Cl; R₂ is selected from the group consisting of: phenyl and substituted phenyl; R₃ is selected from the group consisting of: phenyl and substituted phenyl; X is S; Y₁ is S; Y₂ is CH; Y₃ is H; and Z is NH₂.

According to one aspect the invention, the compound has the formula:

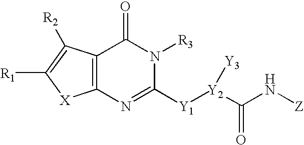

wherein R₁ is selected from the group consisting of: H, halogen, alkyl, substituted alkyl, aryl and substituted aryl; R₂ is selected from the group consisting of: H, alkyl, substituted alkyl, aryl and substituted aryl; R₃ is selected from the group consisting of: H, alkyl, substituted alkyl, aryl, substituted aryl, pyridine, substituted pyridine; X is selected from the group consisting of: S, O and NH; Y₁ is selected from the group consisting of: S, CH₂, NH, O, N-alkyl; Y₂ is selected from the group consisting of: CH, alkyl and substituted alkyl; Y₃ is selected from the group consisting of: H and CH₃; Z is selected from the group consisting of: OH and NH₂; with the provision that when X is S, Y₁ is S, Y₂ is CH, Y₃ is H, R₁ is Me, and R₂ is Me, R₃ can not be H.

According to another aspect of the invention, a compound may have the formula:

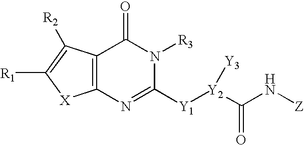

wherein R₁ is selected from the group consisting of: H, alkyl, and substituted alkyl; R₂ is selected from the group consisting of: H, alkyl, substituted alkyl, aryl and substituted aryl, ring system, substituted ring system, carbocyclic ring system, substituted carbocyclic ring system, heterocyclic ring system, and substituted heterocyclic ring system; R₃ is selected from the group consisting of: H, alkyl, substituted alkyl, aryl and substituted aryl, ring system, substituted ring system, carbocyclic ring system, substituted carbocyclic ring system, heterocyclic ring system, and substituted heterocyclic ring system; X is selected from the group consisting of: S, O and NH; Y₁ is selected from the group consisting of: S, CH₂, NH and N-alkyl; Y₂ is selected from the group consisting of: CH, alkyl and substituted alkyl; Y₃ is selected from the group consisting of: H and CH₃; Z is selected from the group consisting of: OH and NH₂; with the provision that when X is S, Y₁ is S, Y₂ is CH, Y₃ is H, R₁ is Me, and R₂ is Me, R₃ can not be Ph or a propylene group (CH₂═CH—CH₂—); and with the provision that when X is S, Y₁ is S, Y₂ is CH, Y₃ is H, R₁ is H, and R₂ is Ph, R₃ can not be Ph. In another aspect, Y₁ in this compound may be O.

According to another aspect of the invention, the compound may have the formula:

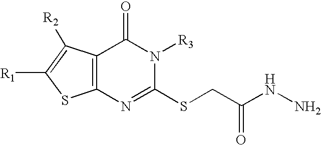

wherein R₁ is selected from the group consisting of: H, Cl, Me, iPr, Ph and substituted Ph; R₂ is selected from the group consisting of: Ph, substituted Ph and H; and R₃ is selected from the group consisting of: Ph and substituted Ph; with the provision that R₃ can not be Ph or a propylene group (CH₂═CH—CH₂—) when R₁ and R₂ are Me; and R₃ can not be Ph when R₁ is H and R₂ is Ph. In one embodiment, R₁ is selected from the group consisting of: H, Cl, Me, iPr, and Ph; R₂ is selected from the group consisting of: 4-F-Ph, Ph, Me, iPr, 4-OMe-Ph, 3-OMe-Ph, 2-OMe-Ph, 2-OH-Ph, 2-(OC₃H₆NEt₂)-Ph, 3-F-Ph, 2-F-Ph, 4-Cy-Ph, 2-CN-Ph, 3-CN-Ph, 4-CN-Ph, nitrile, 3-CF₃-Ph, 3-CF₃—O-Ph and H; and R₃ is selected from the group consisting of: 4-F-Ph, Me, CH₂Ph, 3-Py, Cy, 2OMe-Ph, 3-OMe-Ph, 4-OMe-Ph, 2-Cl-Ph, 3-Cl-Ph, 4-Cl-Ph, 2-F-Ph, 3-F-Ph, 4-F-Ph, 2,5-di-F-Ph, 3,5-di-F-Ph, 2,6-di-F-Ph, 4-Cy-Ph, 2-CN-Ph, 3-CN-Ph, 4-CN-Ph, 3-CF₃-Ph, 3-CF₃—O-Ph and Ph; with the provision that R₃ can not be Ph or a propylene group (CH₂═CH—CH₂—) when R₁ and R₂ are Me; and with the provision that R₃ can not be Ph when R₁ is H and R₂ is Ph. In another embodiment, R₁ is selected from the group consisting of: H, Cl or Me; k₂ is selected from the group consisting of: Ph, 3-OMe-Ph, 2-OMe-Ph, and 2-F-Ph; and R₃ is 2-F-Ph or 3-F-Ph.

According to another aspect of the invention, the compound may have the formula:

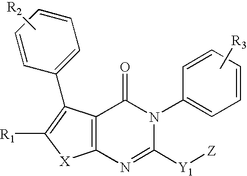

wherein R₁ is selected from the group consisting of: H, halogen and Me; R₂ is selected from the group consisting of: H, 4-F, and 2-F; R₃ is selected from the group consisting of: H, 4-F, and 3-F; X is selected from the group consisting of: S, O and NH; Y₁ is selected from the group consisting of: S, O, NH and NMe; Z is selected from the group consisting of: CH₂C(O)NHNH₂, CH₂CH₂C(O)NHNH₂, CH(Me)C(O)NHNH₂,CH₂C(O)NMeNH₂,CH₂C(O)NHNHMe, CH₂CO₂H, CH₂CO₂Et, CH₂C(O)NHMe, CH₂C(O)NH₂, CH₂C(O)NHOH, and CH₂C(O)CH₂NH₂. In one embodiment, R₁ is Cl or F.

According to another aspect of the invention, the compound may have the formula:

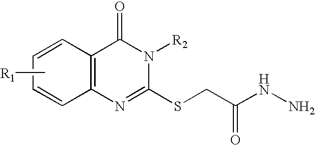

wherein R₁ is selected from the group consisting of: H, and Ph; and R₂ is Ph or 3-F-Ph.

According to another aspect of the invention, the compound may have the formula:

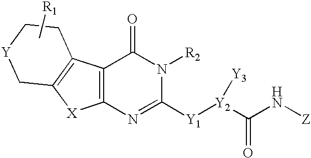

wherein Y is selected from the group consisting of: CH₂, N-Boc, NH, NMe, N-alkyl; and R₁ is selected from the group consisting of: H, Me, Ph, alkyl, arylalkyl, t-butyl, and CH₂Ph; R₂ is selected from the group consisting of: alkyl, substituted alkyl, aryl, substituted aryl; pyridine, and substituted pyridine; X is selected from the group consisting of: S, O and NH; Y₁ is selected from the group consisting of: S, CH₂, O, NH, N-alkyl; Y₂ is selected from the group consisting of: CH, alkyl and substituted alkyl; Y₃ is selected from the group consisting of: H and CH₃; and Z is selected from the group consisting of: OH and NH₂.

Aspects of the invention are based, in part, on the discovery that the series of compounds described herein act as inhibitors of tissue transglutaminase. According to the invention, these compounds, and certain derivatives thereof, are useful to treat diseases such as celiac disease, Alzheimer's disease, Parkinson's disease and Huntington's disease.

In one aspect, the present invention provides compounds and pharmaceutical preparations that are useful for treating disorders associated with tissue transglutaminase.

According to one aspect of the invention, a method of treating a transglutaminase-associated disorder is provided comprising administering to a subject having the transglutaminase-associated disorder a compound of the formula:

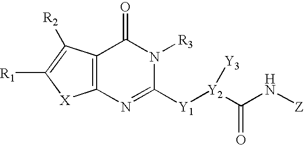

wherein R₁ is selected from the group consisting of: H, halogen, alkyl, substituted alkyl, aryl and substituted aryl; R₂ is selected from the group consisting of: H, alkyl, substituted alkyl, aryl and substituted aryl; R₃ is selected from the group consisting of: H, alkyl, substituted alkyl, aryl, substituted aryl, pyridine, and substituted pyridine; X is selected from the group consisting of: S, O and NH; Y₁ is selected from the group consisting of: S, CH₂, NH, O and N-alkyl; Y₂ is selected from the group consisting of: CH, alkyl and substituted alkyl; Y₃ is selected from the group consisting of: H and CH₃; Z is selected from the group consisting of: OH and NH₂; and wherein the compound is administered in an amount effective to treat the transglutaminase-associated disorder.

According to another aspect of the invention, a method of treating a transglutaminase-associated disorder is provided comprising administering to a subject having the tissue transglutaminase disorder a compound of the formula:

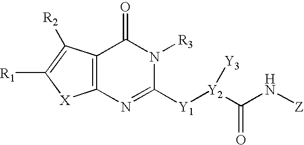

wherein R₁ is selected from the group consisting of: H, alkyl, and substituted alkyl; R₂ is selected from the group consisting of: H, alkyl, substituted alkyl, aryl and substituted aryl, ring system, substituted ring system, carbocyclic ring system, substituted carbocyclic ring system, heterocyclic ring system, and substituted heterocyclic ring system; R₃ is selected from the group consisting of: H, alkyl, substituted alkyl, aryl and substituted aryl, ring system, substituted ring system, carbocyclic ring system, substituted carbocyclic ring system, heterocyclic ring system, and substituted heterocyclic ring system, X is selected from the group consisting of: S, O and NH; Y₁ is selected from the group consisting of: S, CH₂, NH and N-alkyl; Y₂ is selected from the group consisting of: CH, alkyl and substituted alkyl; Y₃ is selected from the group consisting of: H and CH₃; Z is selected from the group consisting of: OH and NH₂; and wherein the compound is administered in an amount effective to treat the transglutaminase-associated disorder. In another aspect, Y₁ in this compound may be O.

According to another aspect of the invention, a method of treating a transglutaminase-associated disorder is provided the method comprising administering to a subject having the transglutaminase-associated disorder a compound of the formula:

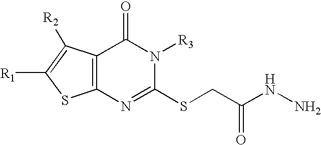

wherein R₁ is selected from the group consisting of: H, Cl, Me, iPr, Ph and substituted Ph; R₂ is selected from the group consisting of: Ph, substituted Ph and H; R₃ is selected from the group consisting of: Ph and substituted Ph; and wherein the compound is administered in an amount effective to treat the transglutaminase- associated disorder. In one embodiment, R₁ is selected from the group consisting of: H, Cl, Me, iPr, and Ph; R₂ is selected from the group consisting of: 4-F-Ph, Ph, Me, iPr, 4-OMe-Ph, 3-OMe-Ph, 2-OMe-Ph, 2-OH-Ph, 2-(OC₃H₆NEt₂)-Ph, 3-F-Ph, 2-F-Ph, 4-Cy-Ph, 2-CN-Ph, 3-CN-Ph, 4-CN-Ph, nitrile, 3-CF₃-Ph, 3-CF₃—O-Ph and H; and R₃ is selected from the group consisting of: 4-F-Ph, Me, CH₂Ph, 3-Py, Cy, 2OMe-Ph, 3-OMe-Ph, 4-OMe-Ph, 2-Cl-Ph, 3-Cl-Ph, 4-Cl-Ph, 2-F-Ph, 3-F-Ph, 4-F-Ph, 2,5-di-F-Ph, 3,5-di-F-Ph, 2,6-di-F-Ph, 4-Cy-Ph, 2-CN-Ph, 3-CN-Ph, 4-CN-Ph, 3-CF₃-Ph, 3-CF₃—O-Ph and Ph; with the provision that R₃ can not be Ph or a propylene group (CH₂═CH—CH₂—) when R₁ and R₂ are Me; and with the provision that R₃ can not be Ph when R₁ is H and R₂ is Ph. In yet another embodiment, R₁ is selected from the group consisting of: H, Cl or Me; R₂ is selected from the group consisting of: Ph, 3-OMe-Ph, 2-OMe-Ph, and 2-F-Ph; and R₃ is 2-F-Ph or 3-F-Ph.

According to another aspect of the invention, a method of treating a transglutaminase-associated disorder is provided comprising administering to a subject having the transglutaminase-associated disorder a compound of the formula:

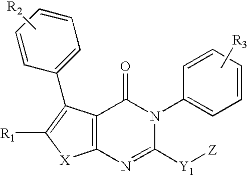

wherein R₁ is selected from the group consisting of: H, halogen and Me; R₂ is selected from the group consisting of: H, 4-F, and 2-F; R₃ is selected from the group consisting of: H, 4-F, and 3-F; X is selected from the group consisting of S, O and NH; Y₁ is selected from the group consisting of: S, O, NH and NMe; Z is selected from the group consisting of: CH₂C(O)NHNH₂, CH₂CH₂C(O)NHNH₂, CH(Me)C(O)NHNH₂, CH₂C(O)NMeNH₂, CH₂C(O)NHNHMe, CH₂CO₂H, CH₂CO₂Et, CH₂C(O)NHMe, CH₂C(O)NH₂, CH₂C(O)NHOH, and CH₂C(O)CH₂NH₂; and wherein the compound is administered in an amount effective to treat the transglutaminase-associated disorder. In one embodiment, R₁ is Cl or F.

According to another aspect of the invention, a method of treating a transglutaminase-associated disorder is provided comprising administering to a subject having the transglutaminase-associated disorder a compound of the formula:

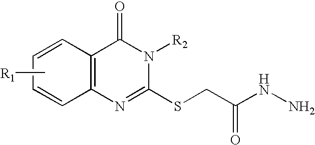

wherein R₁ is selected from the group consisting of: H, and Ph; and R₂ is Ph or 3-F-Ph; and wherein the compound is administered in an amount effective to treat the transglutaminase-associated disorder.

According to another aspect of the invention, a method of treating a transglutaminase-associated disorder is provided comprising administering to a subject having the transglutaminase-associated disorder a compound of the formula:

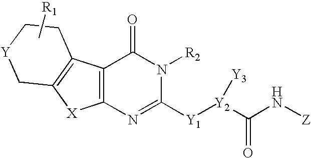

wherein Y is selected from the group consisting of: CH₂, N-Boc, NH, NMe, N-alkyl; and R₁ is selected from the group consisting of: H, Me, Ph, alkyl, arylalkyl, t-butyl, and CH₂Ph; R₂ is selected from the group consisting of: alkyl, substituted alkyl, aryl, substituted aryl; pyridine, and substituted pyridine; X is selected from the group consisting of: S, O and NH; Y₁ is selected from the group consisting of: S, CH₂, O, NH, and N-alkyl; Y₂ is selected from the group consisting of: CH, alkyl and substituted alkyl; Y₃ is selected from the group consisting of: H and CH₃; Z is selected from the group consisting of: OH and NH₂ and wherein the compound is administered in an amount effective to treat the transglutaminase-associated disorder.

Transglutaminases are a family of Ca⁺²-dependent enzymes that catalyze the formation of isopeptide bonds between the carboxamide group of protein/peptide-bound glutamine residues and the ε-amino group of protein/peptide-bound lysine residues to form N^{ε}-(γ-L-glutamyl)-L-lysine cross links with loss of ammonia.

Transglutaminases are normally expressed at low levels in many different tissues. Tissue transglutaminases are inappropriately activated in a variety of pathologies including neurodegenerative diseases, CAG-expansion diseases, autoimmune disease and other conditions (Kim et al., Neurochemistry International 40 (2002) 85).

Transglutaminase-associated disorders are neurodegenerative diseases including, but not limited to: Parkinson's disease, Alzheimer's disease, and progressive supranuclear palsy. In one embodiment of the invention, the compounds of the invention are used for treating a transglutaminase-associated neurodegenerative disorder that is Parkinson's disease or Alzheimer's disease.

Certain CAG-expansion disorders are transglutaminase-associated disorders including but not limited to: spinocerebellar ataxia type 1, spinocerebellar ataxia type 2, Machado-Joseph disorder, spinocerebellar ataxia type 6, spinocerebellar ataxia type 7, spinocerebellar ataxia type 12, spinocerebellar ataxia type 17, spinobulbar muscular atrophy, dentatorubral-pallidoluysian atrophy and Huntington's disease. In one embodiment, of the invention, the compounds of the invention are used for treating Huntington's disease.

Certain autoimmune disorders are associated with tissue transglutaminase including, but not limited to: hepatitis, hemolytic anemia, myasthenia, subepidermal blisters, multiple sclerosis, lupus, necrobiosis lipoidica, myasthenia gravis, bullous pemphigoid, Goodpasture disease, rheumatoid arthritis, amyloid lateral sclerosis, inclusion body myositis and celiac spru. In one embodiment, compounds of the invention may be used for treating celiac spru.

Compounds of the invention may be small organic molecules. The term “alkyl” refers to the radical of saturated aliphatic groups, including straight-chain alkyl groups, branched-chain alkyl groups, cycloalkyl (alicyclic) groups, carbocyclic groups, carbon ring systems, alkyl substituted cycloalkyl groups, and cycloalkyl substituted alkyl groups. In certain embodiments, a straight chain or branched chain alkyl has 12 or fewer carbon atoms in its backbone (e.g., C₁-C₁₂ for straight chain, C₃-C₁₂ for branched chain), and more preferably 6 or fewer, and even more preferably 4 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure, and even more preferably from one to four carbon atoms in its backbone structure. Likewise, “lower alkenyl” and “lower alkynyl” have similar chain lengths. Preferred alkyl groups are lower alkyls. In certain embodiments, a substituent designated herein as alkyl is a lower alkyl.

As used herein, the term “halogen” designates —F, —Cl, —Br or —I; the term “sulfhydryl” means —SH; and the term “hydroxyl” means —OH.

The term “methyl” refers to the monovalent radical —CH₃, and the term “methoxyl” refers to the monovalent radical —CH₂OH.

The term “aralkyl” or “arylalkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as heterocyclic ring systems, aryl heterocycles or heteroaromatics. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” or “heteroaryl” refer to 3-to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, heterocyclyl, aromatic or heteroaromatic moiety, —CF₃, —CN, or the like.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit provision that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, heterocyclyl, aromatic or heteroaromatic moiety, —CF₃, —CN, or the like. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that the compounds of the invention can be synthesized by the utilization of appropriate protecting groups as it is appreciated in the art of organic synthesis. The protective groups include, but are not limited to N-Boc (N-tert-butoxycarbonyl).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, the present invention relates to a compound represented by any of the structures outlined herein, wherein the compound is a single stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as inhibitors of cellular necrosis), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants, which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

In another aspect, the present invention provides pharmaceutically acceptable compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Examples of such formulations include, but are not limited to DMSO, 10 mM DMSO, 8% hydroxypropyl-beta-cyclodextrin in PBS, propylene glycol, etc.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

As set out herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J Pharm. Sci. 66:1-19.)

Pharmaceutically acceptable salts of the subject compounds may include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants also can be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention also may be administered as a bolus, electuary or paste.

In solid dosage forms of the invention for oral administration (capsules, tablets, pills, dragees, powders, granules and the like), the active ingredient may be mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions of the present invention, such as dragees, capsules, pills and granules, optionally may be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter (and/or a filter that retains viruses and/or other microorganisms), or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the invention include pharmaceutically acceptable emulsions, microemulsions, solutions, suspensions, syrups and elixirs. In addition to the active ingredient, a liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions also can include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of pharmaceutical compositions of the invention for rectal or vaginal administration may be presented as a suppository, which may be prepared by mixing one or more compounds of the invention with one or more suitable non-irritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention that are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In another aspect, the present invention relates to a method of treating a disorder associated with tissue transglutaminase. In particular, the invention provides methods for treating a disorder associated with tissue transglutaminase in a mammal, comprising the step of administering to said mammal a therapeutically effective amount of a compound or therapeutic preparation of the present invention. In certain embodiments, the disorder associated with tissue transglutaminase is an autoimmune disorder such as celiac spru. In other embodiments, the disorder is a neurological disease, such as Parkinson's disease (PD), Alzheimer's disease or Huntington's disease. In other embodiments the disorder is a tissue transglutaminase disease of organs including but not limited to brain, heart, kidney, and liver. In certain embodiments, the mammal is a primate, canine or feline subject. In other embodiments, the mammal is a human subject.

An additional aspect of the invention relates to the use of the compounds described herein for treating necrotic cell diseases associated with tissue transglutaminase including trauma, ischemia, stroke, cardiac infarction, infection and sepsis. Trauma is any physical damage to the body caused by violence, accident, fracture etc. Ischemia refers to a cardiovascular disorder characterized by a low oxygen state usually due to the obstruction of the arterial blood supply or inadequate blood flow leading to hypoxia in the tissue. Stroke refers to cardiovascular disorders caused by a blood clot or bleeding in the brain, most commonly caused by an interruption in the flow of blood in the brain as from clot blocking a blood vessel, and in certain embodiments of the invention the term stroke refers to ischemic stroke or hemorrhagic stroke. Myocardial infarction refers to a cardiovascular disorder characterized by localized necrosis resulting from obstruction of the blood supply.

The compounds of the invention can be tested in standard animal models of stroke using protocols described by Hara, H., et al. Proc Natl Acad Sci USA, 1997. 94(5): 2007-12.

Accordingly, the compounds of the invention can be used in combination with agents for the treatment of cardiovascular disorders. Agents for treating cardiovascular disorders include compounds selected from the group consisting of anti-inflammatory agents, anti-thrombotic agents, anti-platelet agents, fibrinolytic agents, lipid reducing agents, direct thrombin inhibitors, glycoprotein IIb/IIIa receptor inhibitors, agents that bind to cellular adhesion molecules and inhibit the ability of white blood cells to attach to such molecules (e.g. anti-cellular adhesion molecule antibodies), calcium channel blockers, beta-adrenergic receptor blockers, cyclooxygenase-2 inhibitors, angiotensin system inhibitors, and/or any combinations thereof.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. A therapeutically effective amount for treating a neurological disorder is an amount sufficient to inhibit necrosis in at least a subset of cells that were exposed to a cell-death initiating event. Accordingly, a therapeutically effective amount prevents or minimizes disease progression associated with cellular necrosis. Disease progression can be monitored relative to an expected disease progression that is based on population studies, controlled observations in individuals, or a combination of both.

In certain embodiments, a compound or pharmaceutical preparation is administered orally. In other embodiments, the compound or pharmaceutical preparation is administered intravenously. Alternative routes of administration include sublingual, intramuscular, and transdermal administrations.

In certain embodiments, the present invention relates to ligands for inhibiting cell death, wherein the ligands are represented by any of the structures outlined above, and any sets of definitions associated with one of those structures. In certain embodiments, the ligands of the present invention are inhibitors of cell death. In any event, the ligands of the present invention preferably exert their effect on inhibiting cell death at a concentration less than about 50 micro molar, more preferably at a concentration less than about 10 micro molar, and most preferably at a concentration less than 1 micro molar.

The compounds of the invention can be tested in standard protocols such as described by Case et al., Biochemistry 2003, 42, 9466-9481. The compounds of the invention can be tested in animal models of disease, such as Huntington's disease (Brouillet, E. Funct. Neurol. 2000, 15, 239-251) and celiac disease (Gregersen, J. W. et al Tissue Antigens 2004, 63, 383-94).

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier. The compounds of the invention are of substantial purity, i.e. substantially free of reaction side-products, for example 0.1% to 99.5% pure, and more preferably, 0.5% to 90% pure.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In certain embodiments, oral administrations may be preferred.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts. A daily, weekly, or monthly dosage (or other time interval) can be used.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In general, a suitable daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition).

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: oral administration, for example, drenches (aqueous or non-aqueous solutions or suspensions), tablets, boluses, powders, granules, pastes for application to the tongue; parenteral administration, for example, by subcutaneous, intramuscular or intravenous injection as, for example, a sterile solution or suspension; topical application, for example, as a cream, ointment or spray applied to the skin, lungs, or oral cavity; or intravaginally or intrarectally, for example, as a pessary, cream or foam; sublingually; ocularly; transdermally; nasally; pulmonary or to other mucosal surfaces.

Compounds according to the invention may be formulated for administration in any convenient way for use in human or veterinary medicine, by analogy with other pharmaceuticals.

The terms “treatment” and “treating” are intended to encompass also prophylaxis, therapy and cure. Accordingly, in one aspect, a treatment includes preventing or delaying or slowing the onset of a disease, condition, or disorder (e.g. the symptoms associated with the disease, condition, or disorder). In another aspect, a treatment includes treating (e.g. minimizing or reducing or slowing the development or reversing) an existing disease, condition, or disorder (e.g. the symptoms associated with the disease, condition, or disorder). In one embodiment, a treatment provides a cure for a disease, condition, or disorder. The patient receiving this treatment may be any animal in need of such treatment, including primates, in particular humans, and other mammals such as equines, cattle, swine and sheep; and poultry and pets in general.

Compounds of the invention described herein can be combined with other therapeutic agents, such as memantine, tacrine, donepezil, rivastigmine, galantamine, levodopa, ropinirole, pramipexole, and pergolide. The compounds of the invention and other therapeutic agent may be administered simultaneously or sequentially. When the other therapeutic agents are administered simultaneously they can be administered in the same or separate formulations, but are administered at the same time. The other therapeutic agents are administered sequentially with one another and with compounds of the invention, when the administration of the other therapeutic agents and the compounds of the invention is temporally separated. The separation in time between the administration of these compounds may be a matter of minutes or it may be longer. Other therapeutic agents include but are not limited to apoptosis inhibitors, PARP poly(ADP-ribose) polymerase inhibitors, Src inhibitors, anti- inflammatory agents, calcium channel blockers, COX-2 inhibitors etc.

In one aspect of the invention, compounds can be administered in combination with compounds that are apoptosis inhibitors. The term “apoptosis inhibitor” refers to compounds that inhibit apoptosis, including but not limited to reversible and irreversible caspase inhibitors. An example of an apoptosis inhibitor includes zVAD (N-benzyloxycarbonyl-Val-Ala-Asp-(OMe) fluoromethyl ketone), IETD (N-acetyl-Ile-Glu-Thr-Asp-al) (SEQ ID NO.:1), YVAD (N-benzyloxycarbonyl-Tyr-Val-Ala-Asp-(OMe) fluoromethyl ketone) (SEQ ID NO.:2), DEVD (N-[2-(6-hydroxy-3-oxo-3H-xanthen-9-yl)benzoyl]-L-α-aspartyl-L-α-glutamyl-N-[(1S)-1-(carboxymethyl)-3 -fluoro-2-oxopropyl]-L-Valinamide (SEQ ID NO.:3)), and LEHD (N-acetyl-Leu-Glu-His-Asp-al) (SEQ ID NO. :4).

In some embodiments, the compounds of the invention are administered in combination with PARP poly(ADP-ribose) polymerase inhibitors. Non-limiting examples of PARP inhibitors include 6(5H)-Phenanthridinone, 4-Amino-1,8-naphthalimide, 1,5-Isoquinolinediol, and 3-Aminobenzamide.

In yet other embodiments, the compounds of the invention are administered in combination with Src inhibitors. Src proteins are mammalian cytoplasmic tyrosine kinases that play an extensive role in signal transduction. Examples of Src inhibitors include but are not limited to: PP1(1-(1,1-dimethylethyl)-1-(4-methylphenyl)-1H-pyrazolo[3,4-d]pyrimidin-4-amine), PP2 (3-(4-chlorophenyl)-1-(1,1-dimethylethyl)-1H-pyrazolo[3,4-d]pyrimidin-4-amine), damnacanthal (3-hydroxy-1-methoxy- 2-anthraquinonecarboxaldehyde), and SU-5565.

In one aspect of the invention, one or more compounds can be administered in combination with compounds that are anti-inflammatory agents.

In one embodiment, a transglutaminase inhibitor may be administered in combination with aspirin.

“Anti-inflammatory” agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

In one aspect of the invention, compounds can be administered in combination with compounds that are calcium channel blockers. “Calcium channel blockers” are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogeneous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, amrinone, amlodipine, bencyclane, felodipine, fendiline, flunarizine, isradipine, nicardipine, nimodipine, perhexilene, gallopamil, tiapamil and tiapamil analogues (such as 1993RO-11-2933), phenytoin, barbiturates, and the peptides dynorphin, omega-conotoxin, and omega-agatoxin, and the like and/or pharmaceutically acceptable salts thereof.

Cyclooxygenase-2 (COX-2) is a recently identified new form of a cyclooxygenase. “Cyclooxygenase” is an enzyme complex present in most tissues that produces various prostaglandins and thromboxanes from arachidonic acid. Non-steroidal, anti-inflammatory drugs exert most of their anti-inflammatory, analgesic and antipyretic activity and inhibit hormone-induced uterine contractions and certain types of cancer growth through inhibition of the cyclooxygenase (also known as prostaglandin G/H synthase and/or prostaglandin-endoperoxide synthase). Initially, only one form of cyclooxygenase was known, the “constitutive enzyme” or cyclooxygenase-1 (COX-1). It and was originally identified in bovine seminal vesicles.

Cyclooxygenase-2 (COX-2) has been cloned, sequenced and characterized initially from chicken, murine and human sources (See, e.g., U.S. Pat. No. 5,543,297, issued Aug. 6, 1996 to Cromlish , et al., and assigned to Merck Frosst Canada, Inc., Kirkland, Calif., entitled: “Human cyclooxygenase-2 cDNA and assays for evaluating cyclooxygenase-2 activity”). This enzyme is distinct from the COX-1. COX-2, is rapidly and readily inducible by a number of agents including mitogens, endotoxin, hormones, cytokines and growth factors. As prostaglandins have both physiological and pathological roles, it is believed that the constitutive enzyme, COX-1, is responsible, in large part, for endogenous basal release of prostaglandins and hence is important in their physiological functions such as the maintenance of gastrointestinal integrity and renal blood flow. By contrast, it is believed that the inducible form, COX-2, is mainly responsible for the pathological effects of prostaglandins where rapid induction of the enzyme would occur in response to such agents as inflammatory agents, hormones, growth factors, and cytokines. Therefore, it is believed that a selective inhibitor of COX-2 has similar anti-inflammatory, antipyretic and analgesic properties to a conventional non-steroidal anti-inflammatory drug, and in addition inhibits hormone-induced uterine contractions and also has potential anti-cancer effects, but with reduced side effects. In particular, such COX-2 inhibitors are believed to have a reduced potential for gastrointestinal toxicity, a reduced potential for renal side effects, a reduced effect on bleeding times and possibly a decreased potential to induce asthma attacks in aspirin-sensitive asthmatic subjects, and are therefore useful according to the present invention.

A number of selective “COX-2 inhibitors” are known in the art. These include, but are not limited to, COX-2 inhibitors described in U.S. Pat. No. 5,474,995 “Phenyl heterocycles as cox-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat. No. 5,639,780 N-benzyl indol-3-yl butanoic acid derivatives as cyclooxygenase inhibitors”; U.S. Pat. No. 5,677,318 Diphenyl-1,2-3-thiadiazoles as anti-inflammatory agents”; U.S. Pat. No. 5,691,374 “Diaryl-5-oxygenated-2-(5H)-furanones as COX-2 inhibitors”; U.S. Pat. No. 5,698,584 “3,4-diaryl-2-hydroxy-2,5-dihydrofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada, Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenylheterocycles as cyclooxygenase-2 and 5-lipoxygenase inhibitors.”

A number of the above-identified COX-2 inhibitors are prodrugs of selective COX-2 inhibitors, and exert their action by conversion in vivo to the active and selective COX-2 inhibitors. The active and selective COX-2 inhibitors formed from the above-identified COX-2 inhibitor prodrugs are described in detail in WO 95/00501, published Jan. 5, 1995, WO 95/18799, published Jul. 13, 1995 and U.S. Pat. No. 5,474,995, issued Dec. 12, 1995. Given the teachings of U.S. Pat. No. 5,543,297, entitled: “Human cyclooxygenase-2 cDNA and assays for evaluating cyclooxygenase-2 activity,” a person of ordinary skill in the art would be able to determine whether an agent is a selective COX-2 inhibitor or a precursor of a COX-2 inhibitor, and therefore part of the present invention.

The invention also provides combinations of two or more compounds that inhibit tissue transglutaminase. The invention provides combinations of the compounds of the invention with known transglutaminase inhibitors such as cystamine, monodansyl cadaverine, as described in U.S. Pat. No.6,335,690, hereby incorporated by reference in its entirety. The invention also provides combinations of the compounds of the invention with 1,3,4,5-tetramethyl-2-[(2-oxopropyl)thio]imidazolium chloride (L-682777), a peptide comprising the amino acid sequence RKLMEI (SEQ ID NO.:5), a peptide comprising the amino acid sequence GTLAKKLT (SEQ ID NO.:6), a peptide comprising the amino acid sequence SHLRKVFDK (SEQ ID NO.:7), a peptide comprising the amino acid sequence HDMNKVLDL (SEQ ID NO.:8), a peptide comprising the amino acid sequence MQMKKVLDS (SEQ ID NO.:9), a peptide comprising the amino acid sequence KVLD (SEQ ID NO.:10), a peptide comprising the amino acid sequence KVLDPVKG (SEQ ID NO.:11), a peptide comprising the amino acid sequence KVLDGQDP (SEQ ID NO.:12), a peptide comprising the amino acid sequence PVKG (SEQ ID NO.:13), a peptide comprising the amino acid sequence DPVKG (SEQ ID NO.:14), a peptide comprising the amino acid sequence GQDP (SEQ ID NO.:15) and other transglutaminase inhibitors described in PCT Application No: PCT/US2004/00272, WO2004/069175 which is hereby incorporated by reference. The invention also provides combinations of the compounds of the invention with monoamines and diamines such as cystamine, putrescine, gamma-amino benzoic acid (GABA), N-benzyloxy carbonyl, 5-deazp-4-oxonorvaline p-nitrophenylester, glycine methyl ester, CuSO₄, the oral anti-hyperglycemic agent tolbutamide and other transglutaminase inhibitors described in U.S. Pat. No: 6,794,414 which is hereby incorporated by reference.

The invention also provides combinations of one or more compounds that inhibit tissue transglutaminase combined with one or more additional agents or compounds (e.g., other therapeutic compounds for treating a disease, condition, or inflammation).

The invention also provides kits including one or more compounds or combinations of the invention (e.g., the thiophene, thienopyrimidinone, thienopyrimidinone acylhydrazide, or quinazolinone compounds, or combinations thereof). A kit can also include one or more additional agents or compounds described herein. The different components of the kit can be provided in different containers. The kit can be compartmentalized to receive the containers in close confinement. The kit can also contain instructions for using the compounds according to the invention.

As used herein, a kit such as a compartmentalized kit includes any kit in which compounds or agents are contained in separate containers. Illustrative examples of such containers include, but are not limited to, small glass containers, plastic containers or strips of plastic or paper. Particularly preferred types of containers allow the skilled worker to efficiently transfer reagents from one compartment to another compartment such that the samples and reagents are not cross-contaminated and the agents or solutions of each container can be added in a quantitative fashion from one compartment to another. Such containers include, but are not limited to, a container that will accept a compound or combination of compounds and/or other agents of the invention. One or more compounds or agents can be provided as a powder (e.g. lyophilized powder) or precipitate. Such compound(s) can be resuspended prior to administration in a solution that may be provided as part of the kit or separately available. A kit can contain compounds or agents in other forms such as liquids, gels, solids, as described herein. Different compounds and/or agents may be provided in different forms in a single kit.

The term “IC₅₀” means the dose of a drug that produces 50% of its maximum response or effect. Alternatively, “IC₅₀” means the dose that produces a pre- determined response in 50% of test subjects or preparations. The term “IC₅₀” also means the concentration of a drug that produces 50% of its maximum response or effect in a test assay. Alternatively, “IC₅₀” means the effective concentration that produces a pre-determined response in 50% of test assays.

The compounds of the invention inhibit any measurable activity of transglutaminase. The compounds of the invention are considered inhibitors of transglutaminase if they inhibit transglutaminase activity by 5%, 10%, 20%, 30%, 40%, 50%, 60%, 70%, 80%, 90%, 95% or higher. The compounds of the invention affect any measurable biochemical activity further regulated by transglutaminase although the effect on transglutaminase per se might not be identifiable, for example downstream targets in a biological pathway that are activated or deactivated by transglutaminase activity.

The term “structure-activity relationship (SAR)” refers to the way in which altering the molecular structure of drugs alters their interaction with a receptor, enzyme, etc.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

The following examples relate to methods of synthesizing and assaying certain compounds and intermediates of the invention. Aspects of the invention include. The following reactions may be used to obtain compounds of invention.

### Example 1

### Gewald Reaction—Method A

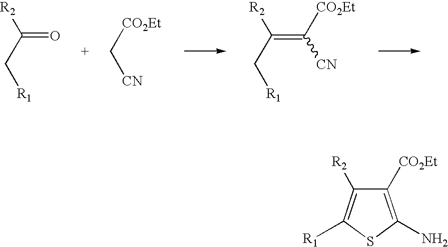

A mixture of ethyl cyanoacetate (1 eq.), ketone (1 eq.), glacial acetic acid (0.8 eq.), ammonium acetate (0.2 eq.) and benzene (5M) was heated at reflux in a flask equipped with a Dean-Stark apparatus and a condenser for 20 h. During that time, the condensed benzene-water mixture was periodically discarded and replaced with dry benzene (2 times). After the reaction mixture was allowed to cool, the residue was poured into a saturated aqueous solution of sodium hydrogencarbonate. The aqueous layer was extracted with dichloromethane and then the combined organic phases were washed with water, dried over anhydrous sodium sulfate, filtered and evaporated. The residue was used directly in the next step.

To a mixture of the crude α,β-unsaturated ester, sulfur (1.1 eq.) and ethanol (1M) was added diethylamine (1.1 eq.). The heterogeneous mixture was vigorously stirred at 50° C. for 18 h, and then the solvent was evaporated. The residue was dissolved in ethyl acetate, washed with brine, evaporated and purified by chromatography on silica gel, eluting with hexane and ethyl acetate, to afford the 2-amino-thiophene-3-carboxylic acid ethyl ester.

This reaction was used to obtain the following components:

2-amino-4-phenylthiophene-3-carboxylic acid ethyl ester

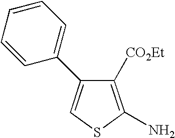

¹H NMR (DMSO-d₆, 500 MHz) δ (ppm): 7.36 (bs, 2H), 7.22-7.31 (m, 5H), 6.16 (s, 1H), 3.93 (q, 2H, J=6.9 Hz), 0.88 (t, 3H, J=6.8 Hz).

2-amino-4-(4-fluorophenyl)thiophene-3-carboxylic acid ethyl ester

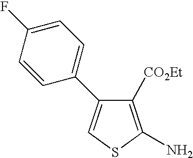

¹H NMR (DMSO-d₆, 500 MHz) δ (ppm): 7.39 (bs, 2H), 7.26 (m, 2H), 7.12 (m, 2H), 6.17 (s, 1H), 3.95 (q, 2H, J=7.0 Hz), 0.91 (t, 3H, J=7.0 Hz).

2-amino-4-(3-fluorophenyl)thiophene-3-carboxylic acid ethyl ester

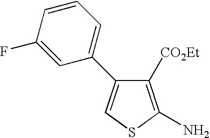

¹H NMR (CDCl₃, 500 MHz) δ (ppm): 7.26 (m, 1H), 7.07 (d, 1H, J=7.5 Hz), 6.97-7.02 (m, 2H), 6.11 (bs, 2H), 6.08 (s, 1H), 4.06 (q, 2H, J=7.2 Hz), 0.96 (t, 3H, J=7.2 Hz).

2-amino-4-(2-fluorophenyl)thiophene-3-carboxylic acid ethyl ester

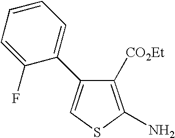

¹H NMR (CDCl₃, 500 MHz) δ (ppm): 7.25-7.31 (m, 2H), 7.10 (td, 1H, J=7.5 Hz, J′=1 Hz), 7.02 (t, 1H, J=9 Hz), 6.13 (bs, 2H), 6.06 (s, 1H), 4.03 (q, 2H, J=7.2 Hz), 0.92 (t, 3H, J=7.0 Hz).

2-Amino-4-(4-methoxyphenyl)thiophene-3-carboxylic acid ethyl ester

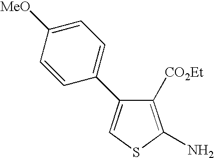

¹H NMR (CDCl₃, 500 MHz) δ (ppm): 7.23 (d, 2H, J=8.5 Hz), 6.85 (d, 2H, J=8.5 Hz), 6.06 (bs, 2H), 6.02 (s, 1H), 4.06 (q, 2H, J=7.2 Hz), 3.83 (s, 3H), 1.00 (t, 3H, J=7.0 Hz).

2-Amino-4-(3-methoxyphenyl)thiophene-3-carboxylic acid ethyl ester

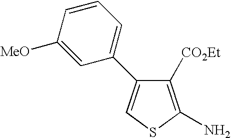

¹H NMR (CDCl₃, 500 MHz) δ (ppm): 7.22 (m, 1H), 6.89 (d, 1H, J=7 Hz), 6.84 (m, 2H), 6.08 (s, 3H), 4.05 (q, 2H, J=7.2 Hz), 3.81 (s, 3H), 0.96 (t, 3H, J=7.0 Hz).

2-Amino-4-(2-methoxyphenyl)thiophene-3-carboxylic acid ethyl ester

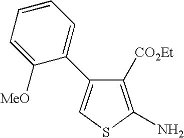

¹H NMR (CDCl₃, 500 MHz) δ (ppm): 7.29 (td, 1H, J=8 Hz, J′=1.5 Hz), 7.19 (dd, 1H, J=7.5 Hz, J′=1.5 Hz), 6.93 (t, 1H, J=7.5 Hz), 6.85 (d, 1H, J=7.5 Hz), 6.08 (s, 1H), 5.94 (bs, 2H), 3.98 (q, 2H, J=7.2 Hz), 3.73 (s, 3H), 0.87 (t, 3H, J=7.0 Hz).

2-Amino-4-isopropylthiophene-3-carboxylic acid ethyl ester

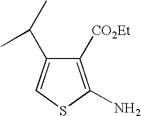

¹H NMR (CDCl₃, 500 MHz) δ (ppm): 6.04 (bs, 2H), 5.89 (s, 1H), 4.30 (q, 2H, J=7.2 Hz), 3.42 (m, 1H), 1.36 (t, 3H, J=7.5 Hz), 1.18 (d, 6H, J=7 Hz).

2-amino-5-methyl-4-phenylthiophene-3-carboxylic acid ethyl ester

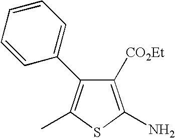

¹H NMR (CDCl₃, 500 MHz) δ (ppm): 7.34-7.27 (m, 3H), 7.15 (m, 2H), 5.94 (bs, 2H), 3.92 (q, 2H, J=7.2 Hz), 2.04 (s, 3H), 0.80 (t, 3H, J=7.0 Hz).

2-Amino-4-(2-fluorophenyl)-5-methylthiophene-3-carboxylic acid ethyl ester

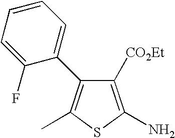

¹H NMR (CDCl₃, 500 MHz) δ (ppm): 7.26-7.31 (m, 1H), 7.14 (td, 1H, J=7.2Hz, J′=2 Hz), 7.11 (t, 1H, J=7.2 Hz), 7.05 (t, 1H, J=8.7 Hz), 5.96 (bs, 2H), 3.88-4.02 (m, 2H), 2.05 (s, 3H), 0.84 (t, 3H, J=7 Hz).

2-Amino-4-(2-fluorophenyl)-5-isopropylthiophene-3-carboxylic acid ethyl ester

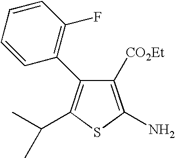

¹H NMR (CDCl₃, 500 MHz) δ (ppm): 7.26-7.30 (m, 1H), 7.14 (td, 1H, J=7.0Hz, J′=2 Hz), 7.10 (td, 1H, J=7.0 Hz, J′=1 Hz), 7.05 (t, 1H, J=9 Hz), 5.99 (bs, 2H), 3.85-3.99 (m, 2H), 2.83 (m, 1H), 1.15 (d, 3H, J=7 Hz), 1.10 (d, 3H, J=7 Hz), 0.80 (t, 3H, J=7 Hz).

### Example 2

### Gewald Reaction—Method B

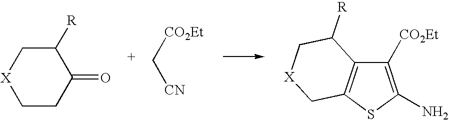

A mixture of ethyl cyanoacetate (1 eq.), ketone (1 eq.), sulfur (1 eq.) and morpholine (1 eq.) in ethanol (1 M) was heated at 50° C. for 18 h. The solvent was evaporated and then the residue was purified by chromatography on silica gel, eluting with hexane and ethyl acetate, to afford the 2-amino-thiophene-3-carboxylic acid ethyl ester.

2-Amino-4-benzyl-4,5,6,7-tetrahydrobenzo[b]thiophene-3-carboxylic acid ethyl ester

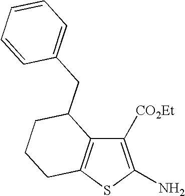

¹H NMR (CDCl₃, 500 MHz) δ (ppm): 7.30 (m, 2H), 7.26 (m, 2H), 7.19 (m, 1H), 5.99 (bs, 2H), 4.32-4.44 (m, 2H), 3.49 (m, 1H), 3.16 (dd, 1H, J=13.5 Hz, J′=3 Hz), 2.48-2.58 (m, 3H), 1.90 (m, 1H), 1.71 (m, 1H), 1.61 (m, 1H), 1.48 (m, 1H), 1.37 (t, 3H, J=7.2 Hz).

2-Amino-4-phenyl-4,5,6,7-tetrahydrobenzo[b]thiophene-3-carboxylic acid ethyl ester

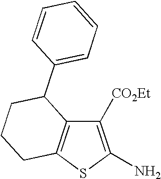

Isolated as a mixture with the starting material. This material was used without further purification.

### Example 3

### Gewald Reaction—Method C

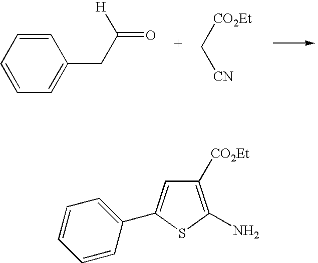

To a mixture of ethyl cyanoacetate (106 μL, 1.1 eq.), phenylacetaldehyde (117 μL, 1 eq.), sulfur (32 mg, 1 eq.), in toluene (5 mL, 0.2 M) was added DBU (150 μL, 1 eq.). The mixture was heated in a pressurized vessel under microwave irradiation at 120° C. for 20 min with stirring and then poured into HCl 1N, extracted with ether, washed with brine, dried over anhydrous Na₂SO₄, filtered and evaporated. The residue was purified by chromatography on silica gel, eluting with hexane and ethyl acetate, to afford the 2-amino-5-phenyl-thiophene-3-carboxylic acid ethyl ester (174 mg, 70%) as a brown solid.

2-amino-5-phenylthiophene-3-carboxylic acid ethyl ester

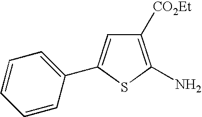

¹H NMR (CDCl₃, 500 MHz) δ (ppm): 7.44 (d, 2H, J=8 Hz), 7.33 (t, 2H, J=7.6 Hz), 7.24 (s, 1H), 7.20 (t, 1H, 7.5 Hz), 6.00 (bs, 2H), 4.30 (q, 2H, J=7.2 Hz), 1.37 (t, 3H, J=7.2 Hz).

### Example 4

### Synthesis of the 5-chlorothiophene Derivative

To a solution of 2-amino-4-(2-fluorophenyl)thiophene-3-carboxylic acid ethyl ester (530 mg, 1 eq.) in chloroform (20 mL, 0.1 M) was added, under argon, triethylamine (280 μL, 1.7 eq.) and trifluoroacetic anhydride (282 μL, 1.2 eq.). The mixture was stirred 1 h at room temperature, then treated with water and extracted with dichloromethane. The combined organic layers were dried over sodium sulfate, filtered and evaporated. The residue was purified by chromatography on silica gel, eluting with hexane and ethyl acetate, to afford 4-(2-fluorophenyl)-2-(2,2,2-trifluoroacetylamino)thiophene-3-carboxylic acid ethyl ester (609 mg, 84%).

4-(2-Fluorophenyl)-2-(2,2,2-trifluoroacetylamino)thiophene-3-carboxylic acid ethyl ester

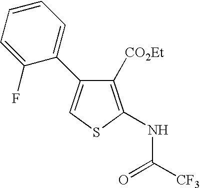

¹H NMR (CDCl₃, 500 MHz) δ (ppm): 12.32 (bs, 1H), 7.35 (m, 1H), 7.28 (td, 1H, J=7.2 Hz, J′=2 Hz), 7.16 (td, 1H, J=7.7 Hz, J′=1 Hz), 7.06 (t, 1H, J=8 Hz), 6.86 (d, 1H, J=1 Hz), 4.14 (q, 2H, J=7.2 Hz), 0.97 (t, 3H, J=7.0 Hz).

At 0° C., under argon, to a solution of trifluoroacetamide (472 mg, 1 eq.) in dichloromethane (6.5 mL, 0.2 M) was added sulfuryl chloride (110 μL, 1.05 eq.). The reaction mixture was stirred for 2 h at room temperature and then poured onto saturated NaHCO₃ and extracted with dichloromethane. The combined organic layers were dried over anhydrous sodium sulfate, filtered and evaporated to afford 5-chloro -4-(2-fluorophenyl)-2-(2,2,2-trifluoroacetylamino)thiophene-3-carboxylic acid ethyl ester (513 mg), which was used without further purification.

5-Chloro-4-(2-fluorophenyl)-2-(2,2,2-trifluoroacetylamino)thiophene-3-carboxylic acid ethyl ester

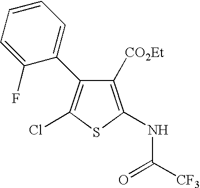

¹H NMR (CDCl₃, 500 MHz) δ (ppm): 12.40 (bs, 1H), 7.39 (m, 1H), 7.25 (td, 1H, J=7.5 Hz, J′=2 Hz), 7.22 (td, 1H, J=7.7 Hz, J′=1 Hz), 7.11 (t, 1H, J=8 Hz), 4.14 (m, 1H), 4.02 (m, 1H), 0.90 (t, 3H, J=7.2 Hz).

At 0° C., to a solution of 5-chloro-4-(2-fluorophenyl)-2-(2,2,2-trifluoroacetylamino)thiophene-3-carboxylic acid ethyl ester (500 mg, 1 eq.) in ethanol (13 mL, 0.1 M) was added (portion wise) sodium borohydride (96 mg, 2 eq.). The reaction mixture was stirred for 1 h at 0° C. and then treated with 1N HCl and extracted with AcOEt. The combined organic layers were washed with saturated NaHCO₃ and brine, dried over anhydrous sodium sulfate, filtered and evaporated. The residue was purified by chromatography on silica gel, eluting with hexane and ethyl acetate, to afford 2-amino-5-chloro-4-(2-fluorophenyl)thiophene-3-carboxylic acid ethyl ester (413 mg) as a pink oil.

2-Amino-5-chloro-4-(2-fluorophenyl)thiophene-3-carboxylic acid ethyl ester

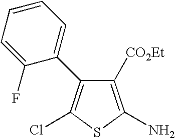

¹H NMR (CDCl₃, 500 MHz) δ (ppm): 7.31-7.38 (m, 1H), 7.23 (m, 1H), 7.15 (m, 1H), 7.08 (m, 1H), 6.11 (bs, 2H), 3.89-4.05 (m, 2H), 0.85 (td, 3H, J=7.5 Hz, J′=2.5 Hz).

### Example 5

### Pyrimidinone Formation

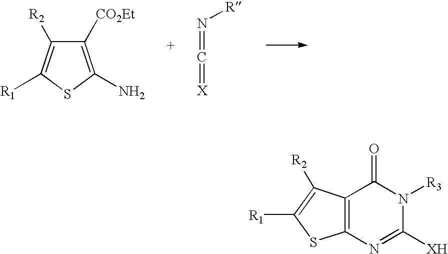

A solution of 2-aminothiophene-3-carboxylic acid ethyl ester (1 eq.), isothiocyanate or isocyanate (1.1 eq.) and pyridine (0.5 M) was heated at 50° C. for 6-24 h. The pyridine was evaporated and a solution of sodium methoxide in methanol (0.5 M, 2.5 eq.) was added. The mixture was stirred at room temperature for 16 h and then poured into 1N HCl. The aqueous layer was extracted with dichloromethane or AcOEt, dried over anhydrous sodium sulfate, filtered, evaporated and recrystallized from ethyl acetate and hexane.

2-Hydroxy-3,5-diphenyl-3H-thieno[2,3-d]pyrimidin-4-one

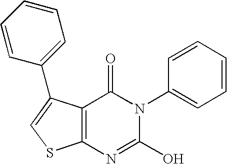

¹H NMR (DMSO-d₆, 500 MHz) δ (ppm): 12.42 (bs, 1H), 7.44 (m, 4H), 7.38 (m, 1H), 7.25-7.34 (m, 5H), 7.05 (s, 1H).

3-(3-Fluorophenyl)-2-mercapto-6-phenyl-3H-quinazolin-4-one

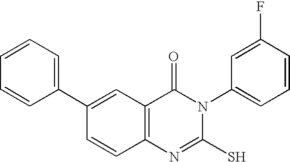

¹H NMR (CDCl₃, 500 MHz) δ (ppm): 10.36 (bs, 1H), 8.37 (d, 1H, J=2 Hz), 7.94 (dd, 1H, J=9 Hz, J′=2 Hz), 7.62 (m, 2H), 7.53 (td, 1H, J=8 Hz, J′=6 Hz), 7.48 (m, 2H), 7.41 (m, 1H), 7.23 (m, 2H), 7.11 (m, 1H), 7.05 (dt, 1H, J=8.5 Hz, J′=2 Hz).

2-Mercapto-3,5-diphenyl-3H-thieno[2,3-d]pyrimidin-4-one

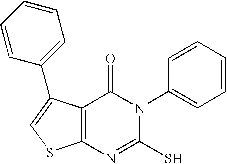

¹H NMR (DMSO-d₆, 500 MHz) δ (ppm): 13.85 (bs, 1H), 7.44 (m, 4H), 7.31-7.38 (m, 4H), 7.23 (m, 3H).

2-Mercapto-3-methyl-5-phenyl-3H-thieno[2,3-d]pyrimidin-4-one

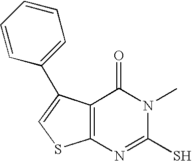

¹H NMR (DMSO-d₆, 500 MHz) δ (ppm): 13.77 (bs, 1H), 7.47 (m, 2H), 7.37 (m, 3H), 7.19 (s, 1H), 3.58 (s, 3H).

3-Benzyl-2-mercapto-5-phenyl-3H-thieno[2,3-d]pyrimidin-4-one

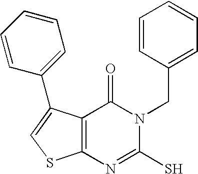

¹H NMR (DMSO-d₆, 500 MHz) δ (ppm): 13.89 (bs, 1H), 7.44 (m, 2H), 7.35 (m, 3H), 7.27 (d, 4H, J=4.5 Hz), 7.22 (s, 1H), 7.21 (m, 1H), 5.59 (s, 2H).

3-Cyclohexyl-2-mercapto-5-phenyl-3H-thieno[2,3-d]pyrimidin-4-one

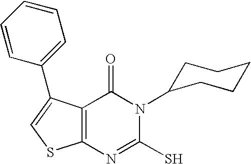

¹H NMR (CDCl₃, 500 MHz) δ (ppm): 13.15 (bs, 1H), 7.46 (m, 2H), 7.38 (m, 3H), 6.71 (s, 1H), 5.72 (m, 1H), 2.49 (m, 2H), 1.81 (d, 2H, J=13 Hz), 1.73 (d, 2H, J=10 Hz), 1.62 (d, 1H, J=13.5 Hz), 1.38 (m, 2H), 1.25 (m, 1H).

2-Mercapto-5-phenyl-3-pyridin-3-yl-3H-thieno[2,3-d]pyrimidin-4-one

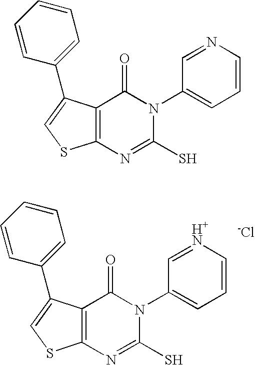

¹H NMR (DMSO-d₆, 500 MHz) δ (ppm): 14.19 (bs, 1H), 8.85 (t, 1H, J=3 Hz), 8.82 (dd, 1H, J=5 Hz, J′=1.5 Hz), 8.77 (m, 2H), 8.20 (d, 1H, J=8.5 Hz), 8.15 (m, 1H), 7.87 (m, 1H), 7.75 (m, 1H), 7.60 (s, 1H), 7.48 (m, 4H), 7.35 (m, 6H).

3-(4-Fluorophenyl)-2-mercapto-5-phenyl-3H-thieno[2,3-d]pyrimidin-4-one

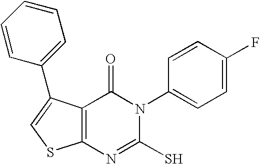

¹H NMR (DMSO-d₆, 500 MHz) δ (ppm): 13.87 (bs, 1H), 7.45 (m, 2H), 7.25-7.35 (m, 7H), 7.23 (s, 1H).

3-(3-Fluorophenyl)-2-mercapto-5-phenyl-3H-thieno[2,3-d]pyrimidin-4-one

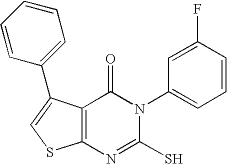

¹H NMR (DMSO-d₆, 500 MHz) δ (ppm): 13.91 (bs, 1H), 7.44-7.51 (m, 3H), 7.34 (m, 3H), 7.23 (m, 3H), 7.12 (d, 1H, J=8 Hz).

3-(2-Fluorophenyl)-2-mercapto-5-phenyl-3H-thieno[2,3-d]pyrimidin-4-one

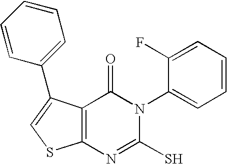

¹H NMR (CDCl₃, 500 MHz) δ (ppm): 11.96 (bs, 1H), 7.46 (m, 3H), 7.23-7.37 (m, 6H). 6.86 (s, 1H).

2-Mercapto-3-(4-methoxyphenyl)-5-phenyl-3H-thieno[2,3-d]pyrimidin-4-one

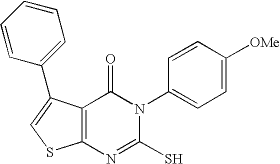

¹H NMR (CDCl₃, 500 MHz) δ (ppm): 12.00 (bs, 1H), 7.48 (m, 2H), 7.33 (m, 3H), 7.17 (d, 2H, J=9 Hz), 7.02 (d, 2H, J=9 Hz), 6.84 (s, 1H), 3.84 (s, 3H).

2-Mercapto-3-(3-methoxyphenyl)-5-phenyl-3H-thieno[2,3-d]pyrimidin-4-one

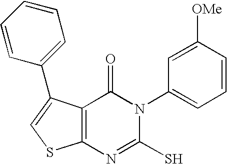

¹H NMR (DMSO-d₆, 500 MHz) δ (ppm): 13.84 (bs, 1H), 7.44 (m, 2H), 7.33 (m, 4H), 7.23 (s, 1H), 6.95 (dd, 1H, J=8 Hz, J′=3 Hz), 6.85 (t, 1H, J=2 Hz), 6.81 (d, 1H, J=8 Hz), 3.74 (s, 3H).

2-Mercapto-3-(2-methoxyphenyl)-5-phenyl-3H-thieno[2,3-d]pyrimidin-4-one

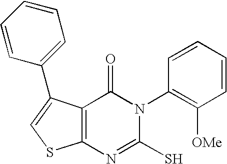

¹H NMR (DMSO-d₆, 500 MHz) δ (ppm): 13.86 (bs, 1H), 7.43 (m, 2H), 7.31-7.38 (m, 4H), 7.23 (s, 1H), 7.17 (dd, 1H, J=8 Hz, J′=1.5 Hz), 7.11 (d, 1H, J=8.5 Hz), 7.00 (td, 1H, J=7.7 Hz, J′=1 Hz), 3.70 (s, 3H).

3-(4-Chlorophenyl)-2-mercapto-5-phenyl-3H-thieno[2,3-d]pyrimidin-4-one

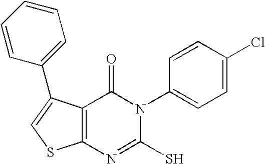

¹H NMR (DMSO-d₆, 500 MHz) δ (ppm): 13.90 (bs, 1H), 7.51 (d, 2H, J=8.5 Hz), 7.45 (m, 2H), 7.33 (m, 3H), 7.29 (d, 2H, J=8.5 Hz), 7.24 (s, 1H).

3-(3-Chlorophenyl)-2-mercapto-5-phenyl-3H-thieno[2,3-d]pyrimidin-4-one

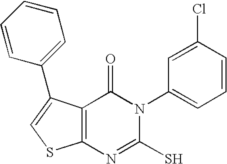

¹H NMR (DMSO-d₆, 500 MHz) δ (ppm): 13.91 (bs, 1H), 7.48 (t, 1H, J=7.7 Hz), 7.45 (m, 3H), 7.43 (t, 1H, J=1.5 Hz), 7.33 (m, 3H), 7.25 (m, 1H), 7.24 (s, 1H).

3-(2-Chlorophenyl)-2-mercapto-5-phenyl-3H-thieno[2,3-d]pyrimidin-4-one

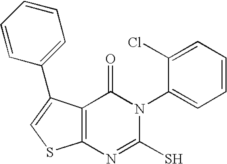

¹H NMR (DMSO-d₆, 500 MHz) δ (ppm): 14.02 (bs, 1H), 7.58 (m, 1H), 7.44 (m, 5H), 7.34 (m, 3H), 7.28 (s, 1H).

3-(2,5-Difluorophenyl)-2-mercapto-5-phenyl-3H-thieno[2,3-d]pyrimidin-4-one

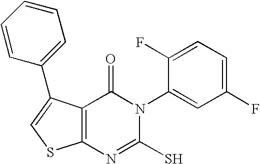

¹H NMR (DMSO-d₆, 500 MHz) δ (ppm): 14.11 (bs, 1H), 7.43 (m, 4H), 7.35 (m, 4H), 7.29 (s, 1H).

3-(3,5-Difluorophenyl)-2-mercapto-5-phenyl-3H-thieno[2,3-d]pyrimidin-4-one

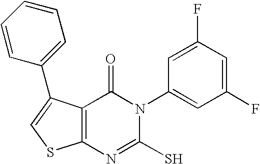

¹H NMR (DMSO-d₆, 500 MHz) δ (ppm): 13.96 (bs, 1H), 7.45 (m, 2H), 7.32 (m, 4H), 7.25 (s, 1H), 7.16 (dd, 2H, J=7.5 Hz, J′=2 Hz).

3-(2,6-Difluorophenyl)-2-mercapto-5-phenyl-3H-thieno[2,3-d]pyrimidin-4-one

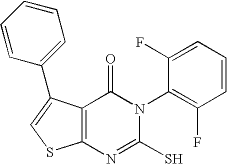

¹H NMR (DMSO-d₆, 500 MHz) δ (ppm): 14.30 (bs, 1H), 7.57 (m, 1H), 7.43 (m, 2H), 7.34 (m, 3H), 7.32 (s, 1H), 7.28 (t, 2H, J=8.2 Hz).

5-(4-Fluorophenyl)-2-mercapto-3-phenyl-3H-thieno[2,3-d]pyrimidin-4-one

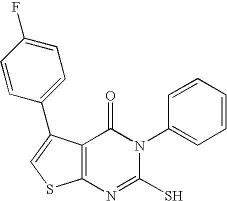

¹H NMR (DMSO-d₆, 500 MHz) δ (ppm): 13.86 (bs, 1H), 7.42-7.50 (m, 4H), 7.36 (t, 1H, J=7.4 Hz), 7.22 (m, 3H), 7.16 (t, 2H, J=9.0 Hz).

3,5-Bis-(4-fluorophenyl)-2-mercapto-3H-thieno[2,3-d]pyrimidin-4-one

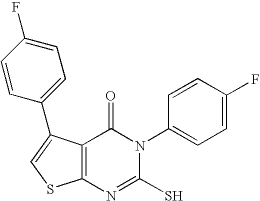

¹H NMR (CDCl₃, 500 MHz) δ (ppm): 11.69 (bs, 1H), 7.44 (m, 2H), 7.20 (m, 4H), 7.03 (t, 2H, J=8.8 Hz), 6.83 (s, 1H).

3,5-Bis-(3-fluorophenyl)-2-mercapto-3H-thieno[2,3-d]pyrimidin-4-one

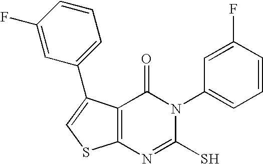

¹H NMR (CDCl₃, 500 MHz) δ (ppm): 11.80 (bs, 1H), 7.49 (td, 1H, J=8 Hz, J′=6 Hz), 7.31 (td, 1H, J=8 Hz, J′=6 Hz), 7.25 (m, 1H), 7.18 (m, 2H), 6.99-7.07 (m, 3H), 6.89 (s, 1H).

3-(3-Fluorophenyl)-5-(2-fluorophenyl)-2-mercapto-3H-thieno[2,3-d]pyrimidin-4-one

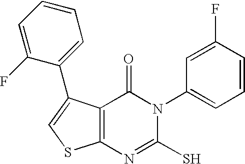

¹H NMR (CDCl₃, 500 MHz) δ (ppm): 11.79 (bs, 1H), 7.48 (td, 1H, J=8 Hz, J′=6 Hz), 7.33 (m, 2H), 7.15 (m, 2H), 7.07 (m, 2H), 7.00 (dt. 1H, J=9 Hz, J′=2 Hz), 6.93 (s, 1H).

3-(4-Fluorophenyl)-2-mercapto-5-methyl-3H-thieno[2,3-d]pyrimidin-4-one

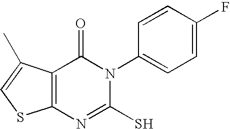

¹H NMR (DMSO-d₆, 500 MHz) δ (ppm): 11.51 (bs, 1H), 7.23 (d, 4H, J=6.4 Hz), 6.56 (m, 1H), 2.46 (d, 3H, J=0.9 Hz).

5 3-(3-Fluorophenyl)-2-mercapto-5-(4-methoxyphenyl)-3H-thieno[2,3-d]pyrimidin-4-one

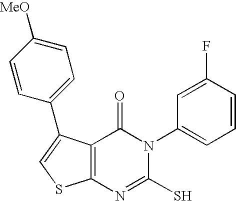

¹H NMR (DMSO-d₆, 500 MHz) δ (ppm): 13.88 (bs, 1H), 7.48 (m, 1H), 7.38 (d, 2H, J=8.5 Hz), 7.23 (m, 2H), 7.15 (s, 1H), 7.12 (d, 1H, J=8 Hz), 6.89 (d, 2H, J=9 Hz), 3.75 (s, 3H).

3-(3-Fluorophenyl)-2-mercapto-5-(3-methoxyphenyl)-3H-thieno[2,3-d]pyrimidin-4-one

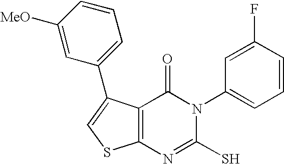

¹H NMR (CDCl₃, 500 MHz) δ (ppm): 11.78 (bs, 1H), 7.48 (td, 1H, J=8 Hz, J′=6 Hz), 7.26 (m, 1H), 7.16 (dd, 1H, J=9 Hz, J′=2.5 Hz), 7.05 (t, 2H, J=7.2 Hz), 7.00 (m, 2H), 6.87 (m, 2H), 3.79 (s, 3H).

3-(3-Fluorophenyl)-2-mercapto-5-(2-methoxyphenyl)-3H-thieno[2,3-d]pyrimidin-4-one

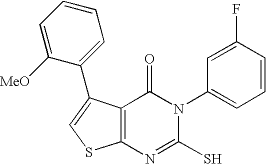

¹H NMR (CDCl₃, 500 MHz) δ (ppm): 11.89 (bs, 1H), 7.46 (td, 1H, J=8 Hz, J′=6.5 Hz), 7.31 (m, 1H), 7.22 (dd, 1H, J=7 Hz, J′=2 Hz), 7.15 (td, 1H, J=8 Hz, J′=2.5 Hz), 7.03 (d, 1H, J=7.5 Hz), 6.95 (m, 2H), 6.89 (d, 1H, J=7.5 Hz), 6.83 (s, 1H), 3.73 (s, 3H).

3-(3-Fluorophenyl)-5-isopropyl-2-mercapto-3H-thieno[2,3-d]pyrimidin-4-one

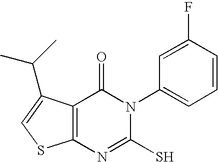

¹H NMR (CDCl₃, 500 MHz) δ (ppm): 11.57 (bs, 1H), 7.52 (td, 1H, J=8 Hz, J′=6 Hz), 7.20 (td, 1H, J=8.5 Hz, J′=2 Hz), 7.05 (m, 1H), 7.02 (dt, 1H, J=8.5 Hz, J′=2 Hz), 6.62 (d, 1H, J=1 Hz), 3.55 (m, 1H), 1.25 (d, 6H, J=7 Hz).

2-Mercapto-3,5-diphenyl-6-methyl-3H-thieno[2,3-d]pyrimidin-4-one

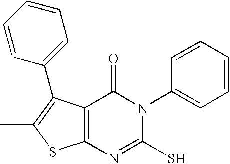

¹H NMR (DMSO-d₆, 500 MHz) δ (ppm): 13.75 (bs, 1H), 7.41 (t, 2H, J=7.3 Hz), 7.28-7.36 (m, 4H), 7.24 (m, 2H), 7.17 (d, 2H, J=7.2 Hz), 2.26 (s, 3H).

2-Mercapto-3,6-diphenyl-3H-thieno[2,3-d]pyrimidin-4-one

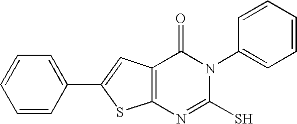

¹H NMR (DMSO-d₆, 500 MHz) δ (ppm): 13.90 (bs, 1H), 7.75 (d, 2H, J=7.5 Hz), 7.70 (s, 1H), 7.35-7.50 (m, 6H), 7.27 (d, 2H, J=7.5 Hz).

6-Chloro-3-(3-fluorophenyl)-5-(2-fluorophenyl)-2-mercapto-3H-thieno[2,3-d]pyrimidin-4-one

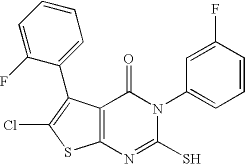

¹H NMR (CDCl₃, 500 MHz) δ (ppm): 12.01 (bs, 1H), 7.46 (m, 1H), 7.37 (m, 1H), 7.31 (m, 1H), 7.15 (m, 3H), 6.93-7.06 (m, 2H).

3-(3-Fluorophenyl)-5-(2-fluorophenyl)-2-mercapto-6-methyl-3H-thieno[2,3-d]pyrimidin-4-one

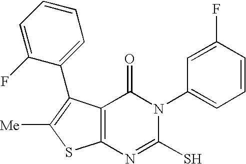

¹H NMR (DMSO-d₆, 500 MHz) δ (ppm): 13.83 (bs, 1H), 7.46 (m, 1H), 7.40 (m, 1H), 7.29 (m, 1H), 7.15-7.23 (m, 4H), 7.06 (m, 1H), 2.23 (d, 3H, J=3 Hz).

3-(3-Fluorophenyl)-2-mercapto-4-oxo-3,5,6,8-tetrahydro-4H-9-thia-1,3,7-triazafluorene-7-carboxylic acid tert-butyl ester

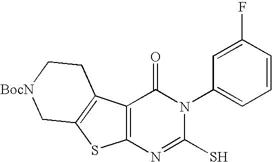

¹H NMR (DMSO-d₆, 500 MHz) δ (ppm): 13.75 (bs, 1H), 7.50 (m, 1H), 7.25 (td, 1H, J=8.5 Hz, J′=2.5 Hz), 7.20 (dt, 1H, J=10 Hz, J′=2 Hz), 7.10 (d, 1H, J=7 Hz), 4.52 (s, 2H), 358 (t, 2H, J=6 Hz), 2.79 (m, 2H), 1.42 (s, 9H).

3-(3-Fluorophenyl)-2-mercapto-5,6,7,8-tetrahydro-3H-benzo[4,5]thieno[2,3-d]pyrimidin-4-one

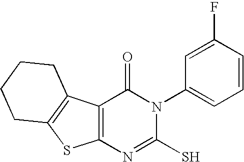

¹H NMR (DMSO-d₆, 500 MHz) δ (ppm): 13.71 (bs, 1H), 7.50 (td, 1H, J=8 Hz, J′=6.5 Hz), 7.25 (td, 1H, J=8.5 Hz, J′=2.5 Hz), 7.20 (dt, 1H, J=9.5 Hz, J′=2 Hz), 7.10 (m, 1H), 2.73 (m, 2H), 2.68 (m, 2H), 1.78 (m, 2H), 1.72 (m, 2H).

3-(3-Fluorophenyl)-2-mercapto-5-methyl-5,6,7,8-tetrahydro-3H-benzo[4,5]thieno[2,3-d]pyrimidin-4-one

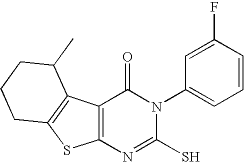

¹H NMR (DMSO-d₆, 500 MHz) δ (ppm): 13.73 (bs, 1H), 7.51 (m, 1H), 7.19-7.27 (m, 2H), 7.11 (m, 1H), 3.16 (m, 1H), 2.73 (m, 1H), 2.61 (m, 1H), 1.81 (m, 2H), 1.72 (m, 1H), 1.63 (m, 1H), 1.44 (m, 3H).

3-(3-Fluorophenyl)-2-mercapto-5-phenyl-5,6,7,8-tetrahydro-3H-benzo[4,5]thieno[2,3-d]pyrimidin-4-one

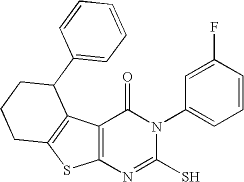

¹H NMR (DMSO-d₆, 500 MHz) δ (ppm): 13.78 (bs, 1H), 7.42 (m, 1H), 7.87-7.22 (m, 8H), 4.48 (d, 1H, J=4.5 Hz), 2.85 (dd, 1H, J=17 Hz), 2.72 (m, 1H), 1.99 (m, 1H), 1.80 (m, 1H), 1.68 (m, 1H), 1.54 (m, 1H).

5-Benzyl-3-(3-fluorophenyl)-2-mercapto-5,6,7,8-tetrahydro-3H-benzo[14,5]thieno[2,3-d]pyrimidin-4-one

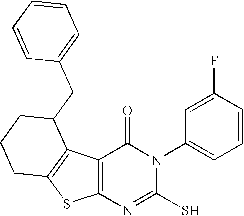

¹H NMR (CDCl₃, 500 MHz) δ (ppm): 11.77 (bs, 1H), 7.54 (m, 1H), 7.29 (d, 2H, J=7.5 Hz), 7.23 (m, 3H), 7.17 (t, 1H, J=7.5 Hz), 7.10 (m, 1H), 7.04 (m, 1H), 3.48 (m, 1H), 3.23 (d, 1H, J=11 Hz), 2.76 (m, 1H), 2.66 (m, 1H), 2.46 (t, 1H, J=12.2 Hz), 1.96 (m, 1H), 1.85 (m, 1H), 1.70 (d, 1H, J=13.5 Hz), 1.47 (m, 1H).

### Example 6

### Synthesis of O and N Analogs

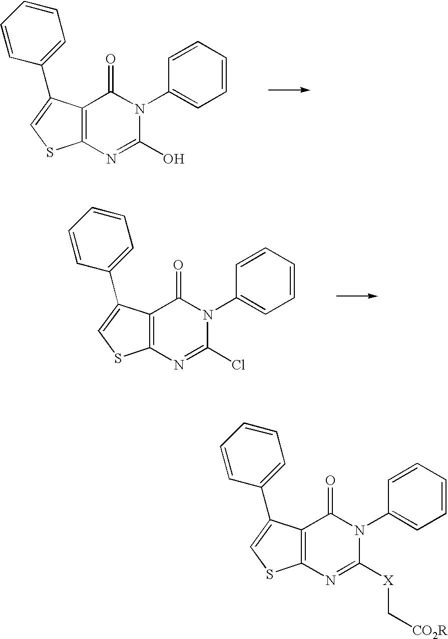

A mixture of 2-hydroxy-3,5-diphenyl-3H-thieno[2,3-d]pyrimidin-4-one (250 mg, 1 eq.) and POCl₃ (5 mL) was heated in a pressurized vessel at 150° C. for 1 h under microwave irradiation with stirring. The reaction mixture was then poured onto ice and the aqueous layer was extracted with AcOEt (3 times). The combined organic layers were washed with saturated NaHCO₃ and brine, dried over anhydrous sodium sulfate, filtered and evaporated to afford 2-chloro-3,5-diphenyl-3H-thieno[2,3-d]pyrimidin-4-one (267 mg), which was used without further purification.

2-Chloro-3,5-diphenyl-3H-thieno[2,3-d]pyrimidin-4-one

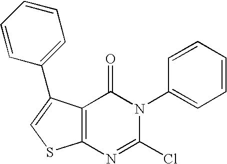

¹H NMR (CDCl₃, 500 MHz) δ (ppm): 7.50 (m, 5H), 7.34 (m, 3H), 7.26 (m, 2H), 7.18 (s, 1H).

Ethyl 2-hydroxyacetate (70 mL, 2.5 eq.) was added under argon at room temperature to a suspension of sodium hydride (30 mg, 60% in mineral oil, 2.5 eq.) in dry THF (1 mL). After 5 min, 2-chloro-3,5-diphenyl-3H-thieno[2,3-d]pyrimidin-4-one (100 mg, 1 eq.) was added and the reaction mixture was heated at reflux for 1.5 h. Next, HCl 1N was added, and the aqueous layer was extracted with CH₂Cl₂. The combined organic layers were dried over anhydrous sodium sulfate, filtered and evaporated. The residue was purified by chromatography on silica gel, eluting with hexane and ethyl acetate, to afford (4-oxo-3,5-diphenyl-3,4-dihydrothieno[2,3-d]pyrimidin-2-yloxy)acetic acid ethyl ester (63 mg, 52%) as a solid.

(4-Oxo-3,5-diphenyl-3,4-dihydrothieno[2,3-d]pyrimidin-2-yloxy)acetic acid ethyl ester

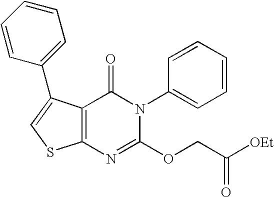

¹H NMR (CDCl₃, 500 MHz) δ (ppm): 7.53 (m, 2H), 7.48 (m, 2H), 7.41 (m, 1H), 7.32 (m, 5H), 6.93 (s, 1H), 4.89 (s, 2H), 4.25 (q, 2H, J=7.2 Hz), 1.30 (t, 3H, J=7 Hz).

A mixture of 2-chloro-3,5-diphenyl-3H-thieno[2,3-d]pyrimidin-4-one (100 mg, 1 eq.), sarcosine ethyl ester hydrochloride (227 mg, 5 eq.), triethylamine (250 μL, 6 eq.), acetonitrile (1.5 mL) and ethanol (0.5 mL) was heated at 60° C. in a closed flask. After 24 h, sarcosine ethyl ester hydrochloride (227 mg, 5 eq.), triethylamine (250 μL, 6 eq.), acetonitrile (0.5 mL) and ethanol (0.25 mL) were added, and the reaction mixture was heated at 60° C. for an additional 6 h. The reaction mixture was diluted with brine and extracted with CH₂Cl₂. The combined organic layers were dried over anhydrous sodium sulfate, filtered and evaporated. The residue was purified by chromatography on silica gel, eluting with hexane and ethyl acetate, to afford [methyl-(4-oxo-3,5-diphenyl-3,4-dihydrothieno[2,3-d]pyrimidin-2-yl)-amino]acetic acid ethyl ester (116 mg, 94%) as an oil.

[Methyl-(4-oxo-3,5-diphenyl-3,4-dihydrothieno[2,3-d]pyrimidin-2-yl) -amino]acetic acid ethyl ester

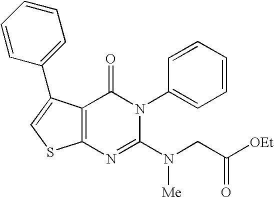

¹H NMR (CDCl₃, 500 MHz) δ (ppm): 7.51 (m, 2H), 7.47 (m, 2H), 7.31-7.38 (m, 5H), 7.28 (m, 1H), 6.80 (s, 1H), 4.18 (q, 2H, J=7.2 Hz), 3.93 (s, 2H), 2.58 (s, 3H), 1.29 (t, 3H, J=7.2 Hz).

A mixture of 2-chloro-3,5-diphenyl-3H-thieno[2,3-d]pyrimidin-4-one (100 mg, 1 eq.), glycine methyl ester hydrochloride (222 mg, 6 eq.), triethylamine (332 μL, 8 eq.), acetonitrile (3 mL) and ethanol (3 mL) was heated at 60° C. in a closed flask. After 48 h, the reaction mixture was diluted with brine and extracted with CH₂Cl₂. The combined organic layers were dried over anhydrous sodium sulfate, filtered and evaporated. The residue was purified by chromatography on silica gel, eluting with hexane and ethyl acetate, to afford a solid mixture of (4-oxo-3,5-diphenyl-3,4-dihydrothieno[2,3-d]pyrimidin-2-ylamino)acetic acid methyl ester and (4-Oxo-3,5-diphenyl-3,4-dihydrothieno[2,3-d]pyrimidin-2-ylamino)acetic acid ethyl ester (82 mg).

(4-Oxo-3,5-diphenyl-3,4-dihydrothieno[2,3-d]pyrimidin-2-ylamino)acetic acid ester

Isolated as a mixture with the starting material. This material was used without further purification.

### Example 7

### Michael Addition

A mixture of 2-mercapto-3,5-diphenyl-6-methyl-3H-thieno[2,3-d]pyrimidin-4-one (100 mg, 1 eq.), methylacrylate (129 μL, 5 eq.), triethylamine (40 μL, 1 eq.) and methanol (2.9 mL, 0.1 M) was heated at 60° C. in a closed reaction tube for 4 h and then at room temperature overnight. The precipitated solid was filtered, yielding 3-(6-methyl-4-oxo-3,5-diphenyl-3,4-dihydrothieno[2,3-d]pyrimidin-2-ylsulfanyl)propionic acid methyl ester (105 mg) as a white solid.

3-(6-Methyl-4-oxo-3,5-diphenyl-3,4-dihydrothieno[2,3-d]pyrimidin-2-ylsulfanyl)propionic acid methyl ester

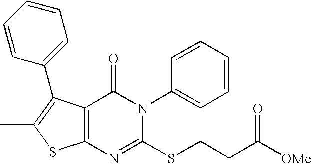

¹H NMR (CDCl₃, 400 MHz) δ (ppm): 7.45 (m, 3H), 7.35 (m, 2H), 7.29 (m, 3H), 7.22 (m, 2H), 3.70 (s, 3H), 3.36 (t, 2H, J=7 Hz), 2.81 (t, 2H, J=7.2 Hz), 2.36 (s, 3H).

### Example 8

### Thiol Alkylation

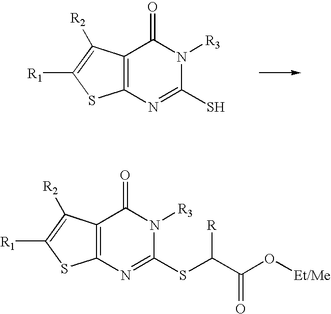

A mixture of heteroaromatic thiol (1 eq.), potassium carbonate (1.2 eq.) and ethyl bromoacetate (1 to 1.5 eq.) or methyl 2-chloropropionate (1.5 eq.) in THF (0.5 M) or THF/DMF (1/1, 0.5 eq.) was heated at 80° C. for 1-16 h. After completion of the reaction as indicated by thin layer chromatography (TLC), the mixture was diluted with water and extracted with dichloromethane. The combined organic layers were dried over anhydrous sodium sulfate, filtered and evaporated. The residue was purified by silica gel chromatography (hexane/ethyl acetate) to give the mercapto-acetic acid derivative.

(4-Oxo-3,5-diphenyl-3,4-dihydrothieno[2,3-d]pyrimidin-2-ylsulfanyl)acetic acid ethyl ester

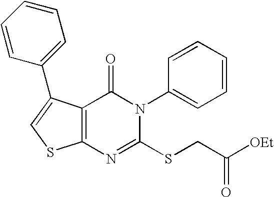

¹H NMR (DMSO-d₆, 500 MHz) δ (ppm): 7.57 (m, 3H), 7.48 (m, 2H), 7.44 (m, 3H), 7.33 (m, 3H), 4.15 (q, 2H, J=6.9 Hz), 3.97 (s, 2H), 1.24 (t, 3H, J=7.0 Hz).

(4-Oxo-3-phenyl-3,4-dihydroquinazolin-2-ylsulfanyl)acetic acid ethyl ester

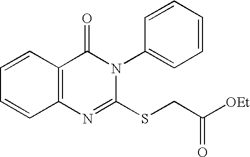

¹H NMR (CDCl₃, 500 MHz) δ (ppm): 8.24 (dd, 1H, J=8 Hz, J′=1.5 Hz), 7.73 (ddd, 1H, J=9 Hz, J′=7.5 Hz, J″=2 Hz), 7.57 (m, 4H), 7.42 (t, 1H, J=7.7 Hz), 7.37 (m, 2H), 4.23 (q, 2H, J=7.2 Hz), 3.92 (s, 2H), 1.31 (t, 3H, J=7 Hz).

[3-(3-Fluorophenyl)-4-oxo-6-phenyl-3,4-dihydroquinazolin-2-ylsulfanyl]acetic acid ethyl ester

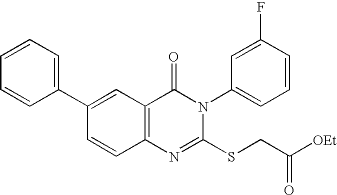

¹H NMR (CDCl₃, 500 MHz) δ (ppm): 8.44 (d, 1H, J=2 Hz), 7.98 (dd, 1H, J=8 Hz, J′=2 Hz), 7.68 (m, 2H), 7.63 (d, 1H, J=8.5 Hz), 7.55 (td, 1H, J=8 Hz, J′=6 Hz), 7.48 (m, 2H), 7.39 (m, 1H), 7.28 (m, 1H), 7.20 (m, 1H), 7.14 (dt, 1H, J=8 Hz, J′=2 Hz), 4.25 (q, 2H, J=7 Hz), 3.94 (s, 2H), 1.32 (t, 3H, J=7.2 Hz).

[3-(4-Fluorophenyl)-4-oxo-5-phenyl-3,4-dihydrothieno[2,3-d]pyrimidin-2-ylsulfanyl]acetic acid ethyl ester (986-58)

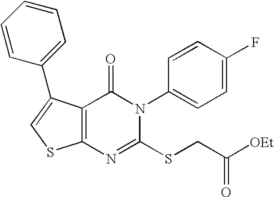

¹H NMR (DMSO-d₆, 500 MHz) δ (ppm): 7.54 (m, 2H), 7.49 (dd, 2H, J=7.5 Hz, J′=1.4 Hz), 7.43 (t, 3H, J=9.2 Hz), 7.30-7.36 (m, 3H), 4.15 (q, 2H, J=7.2 Hz), 3.98 (s, 2H), 1.24 (t, 3H, J=7.2 Hz).

[3-(4-Methoxyphenyl)-4-oxo-5-phenyl-3,4-dihydrothieno[2,3-d]pyrimidin -2-ylsulfanyl]acetic acid ethyl ester

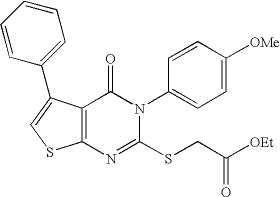

¹H NMR (CDCl₃, 500 MHz) δ (ppm): 7.53 (m, 2H), 7.32 (m, 3H), 7.23 (d, 2H, J=8.5 Hz), 7.01 (m, 3H), 4.24 (q, 2H, J=7.2 Hz), 3.87 (s, 2H), 3.84 (s, 3H), 1.33 (t, 3H, J=7.2 Hz).

[5-(4-Fluorophenyl)-4-oxo-3-phenyl-3,4-dihydrothieno[2,3-d]pyrimidin-2-ylsulfanyl]acetic acid ethyl ester

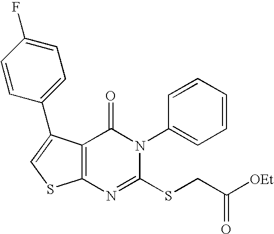

¹H NMR (DMSO-d₆, 500 MHz) δ (ppm): 7.57 (m, 3H), 7.52 (m, 2H), 7.44 (m, 3H), 7.17 (t, 2H, J=9.1 Hz), 4.15 (q, 2H, J=7.2 Hz), 3.97 (s, 2H), 1.24 (t, 3H, J=7.2 Hz).

[3,5-Bis-(4-fluorophenyl)-4-oxo-3,4-dihydrothieno[2,3-d]pyrimidin-2-ylsulfanyl]acetic acid ethyl ester

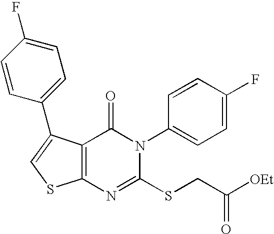

¹H NMR (CDCl₃, 500 MHz) δ (ppm): 7.48 (m, 2H), 7.31 (m, 2H), 7.21 (m, 2H), 7.03 (m, 2H), 7.00 (s, 1H), 4.25 (q, 2H, J=7.2 Hz), 3.89 (s, 2H), 1.33 (t, 3H, J=7.0 Hz).

(3-Methyl-4-oxo-5-phenyl-3,4-dihydrothieno[2,3-d]pyrimidin-2-ylsulfanyl)acetic acid ethyl ester

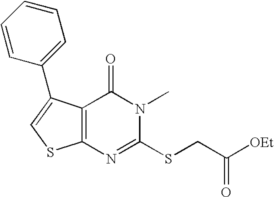

¹H NMR (CDCl₃, 500 MHz) δ (ppm): 7.51 (m, 2H), 7.34-7.42 (m, 3H), 6.96 (s, 1H), 4.27 (q, 2H, J=7.1 Hz), 4.02 (s, 2H), 3.59 (s, 3H), 1.34 (t, 3H, J=7.2 Hz).

(3-Cyclohexyl-4-oxo-5-phenyl-3,4-dihydrothieno[2,3-d]pyrimidin-2-ylsulfanyl)acetic acid ethyl ester

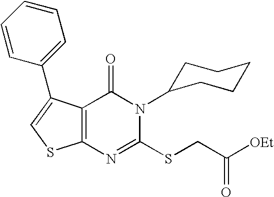

¹H NMR (CDCl₃, 500 MHz) δ (ppm): 7.49 (m, 2H), 7.40 (t, 2H, J=7.2 Hz), 7.35 (t, 1H, J=7.2 Hz), 6.89 (s, 1H), 4.27 (q, 2H, J=7 Hz), 4.10 (bs, 1H), 3.96 (s, 2H), 2.71 (m, 2H), 1.86 (d, 2H, J=13 Hz), 1.76 (m, 2H, J=1.5 Hz), 1.64 (m, 1H), 1.34 (t, 3H, J=7 Hz), 1.29 (m, 3H).

(4-Oxo-5-phenyl-3-pyridin-3-yl-3,4-dihydrothieno[2,3-d]pyrimidin-2-ylsulfanyl)acetic acid ethyl ester

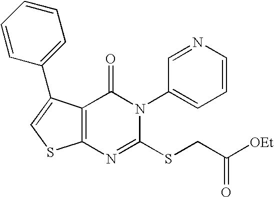

¹H NMR (CDCl₃, 500 MHz) δ (ppm): 8.74 (d, 1H, J=5 Hz), 8.60 (d, 1H, J=2.5 Hz), 7.70 (m, 1H), 7.51 (m, 2H), 7.47 (dd, 1H, J=8.5 Hz, J′=5 Hz), 7.34 (m, 3H), 7.05 (s, 1H), 4.25 (qd, 2H, J=7 Hz, J′=1.5 Hz), 3.97 (d, 1H, J=16 Hz), 3.87 (d, 1H, J=16 Hz), 1.33 (t, 3H, J=7 Hz).

(3-Benzyl-4-oxo-5-phenyl-3,4-dihydrothieno[2,3-d]pyrimidin-2-ylsulfanyl)acetic acid ethyl ester

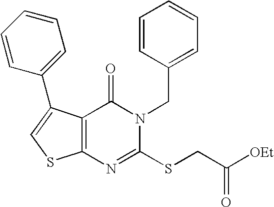

¹H NMR (CDCl₃, 500 MHz) δ (ppm): 7.54 (m, 2H), 7.24-7.42 (m, 8H), 6.98 (s, 1H), 5.38 (s, 2H), 4.24 (q, 2H, J=7 Hz), 3.96 (s, 2H), 1.32 (t, 3H, J=7.2 Hz).

[3-(4-Fluorophenyl)-5-methyl-4-oxo-3,4-dihydrothieno[2,3-d]pyrimidin-2-ylsulfanyl]acetic acid ethyl ester

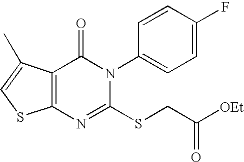

¹H NMR (CDCl₃, 500 MHz) δ (ppm): 7.32 (m, 2H), 7.24 (m, 2H), 6.70 (bd, 1H, J=1.0 Hz), 4.22 (q, 2H, J=7.2 Hz), 3.86 (s, 2H), 2.51 (d, 3H, J=1.4 Hz), 1.31 (t, 3H, J=7.1 Hz).

[3-(3-Fluorophenyl)-5-isopropyl-4-oxo-3,4-dihydrothieno[2,3-d]pyrimidin-2-ylsulfanyl]acetic acid ethyl ester

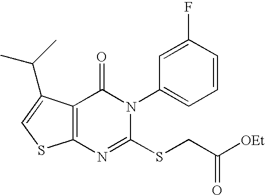

¹H NMR (CDCl₃, 500 MHz) δ (ppm): 7.53 (td, 1H, J=8 Hz, J′=6 Hz), 7.26 (m, 1H), 7.16 (d, 1H, J=8 Hz), 7.10 (dt, 1H, J=8 Hz, J′=2 Hz), 6.78 (s, 1H), 4.23 (q, 2H, J=7.2 Hz), 3.87 (s, 2H), 3.62 (m, 1H), 1.31 (t, 3H, J=7 Hz), 1.26 (d, 6H, J=6.5 Hz).

[3-(3-Fluorophenyl)-5-(4-methoxyphenyl)-4-oxo-3,4-dihydrothieno[2,3-d]pyrimidin-2-ylsulfanyl]acetic acid ethyl ester

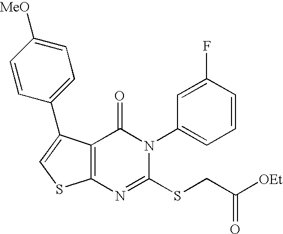

¹H NMR (CDCl₃, 500 MHz) δ (ppm): 7.49 (td, 1H, J=8.5 Hz, J′=6.5 Hz), 7.45 (d, 2H, J=9 Hz), 7.22 (m, 1H), 7.14 (m, 1H), 7.08 (dt, 1H, J=8.5 Hz, J′=2 Hz), 6.97 (s, 1H), 6.88 (d, 2H, J=8.5 Hz), 4.25 (q, 2H, J=7.7 Hz), 3.90 (s, 2H), 3.79 (s, 3H), 1.33 (t, 3H, J=7 Hz).

[3-(3-Fluorophenyl)-5-(3-methoxyphenyl)-4-oxo-3,4-dihydrothieno[2,3-d]pyrimidin-2-ylsulfanyl]acetic acid ethyl ester

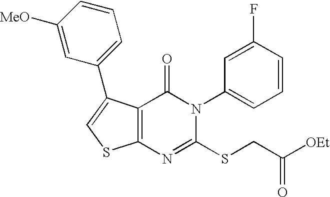

¹H NMR (CDCl₃, 500 MHz) δ (ppm): 7.49 (td, 1H, J=8 Hz, J′=6 Hz), 7.26 (m, 1H), 7.22 (td, 1H, J=8 Hz, J′=2.5 Hz), 7.04-7.15 (m, 5H), 6.86 (dd, 1H, J=8.5 Hz, J′=2.5 Hz), 4.25 (q, 2H, J=7.2 Hz), 3.90 (s, 2H), 3.79 (s, 3H), 1.33 (t, 3H, J=7.2 Hz).

[3-(3-Fluorophenyl)-5-(2-methoxyphenyl)-4-oxo-3,4-dihydrothieno[2,3-d]pyrimidin-2-ylsulfanyl]acetic acid ethyl ester

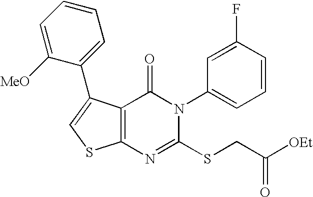

¹H NMR (CDCl₃, 500 MHz) δ (ppm): 7.46 (td, 1H, J=8 Hz, J′=6 Hz), 7.28 (m, 1H), 7.25 (m, 1H), 7.19 (td, 1H, J=8 Hz, J′=2.5 Hz), 7.10 (m, 1H), 7.04 (dt, 1H, J=9 Hz, J′=2.5 Hz), 7.00 (s, 1H), 6.94 (t, 1H, J=7.5 Hz), 6.89 (d, 1H, J=8 Hz), 4.25 (q, 2H, J=7.2 Hz), 3.89 (s, 2H), 3.73 (s, 3H), 1.34 (t, 3H, J=7.2 Hz).

[3,5-Bis-(3-fluorophenyl)-4-oxo-3,4-dihydrothieno[2,3-d]pyrimidin-2-5 ylsulfanyl]acetic acid ethyl ester

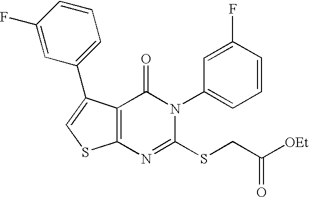

¹H NMR (CDCl₃, 500 MHz) δ (ppm): 7.50 (td, 1H, J=8 Hz, J′=6 Hz), 7.30 (m, 2H), 7.24 (m, 2H), 7.14 (m, 1H), 7.08 (dt, 1H, J=9 Hz, J′=2 Hz), 7.05 (s, 1H), 7.01 (m, 1H), 4.25 (q, 2H, J=7.2 Hz), 3.90 (s, 2H), 1.33 (t, 3H, J=7 Hz).

[3-(3-Fluorophenyl)-5-(2-fluorophenyl)-4-oxo-3,4-dihydrothieno[2,3-d]pyrimidin-2-ylsulfanyl]acetic acid ethyl ester

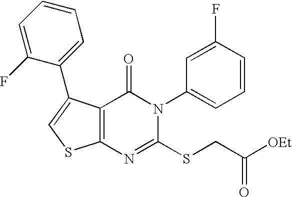

¹H NMR (CDCl₃, 500 MHz) δ (ppm): 7.48 (td, 1H, J=δ Hz, J'═δ Hz), 7.37 (td, 1H, J=8 Hz, J′=1.5 Hz), 7.30 (m, 1H), 7.21 (m, 1H), 7.13 (m, 2H), 7.08 (m, 3H), 4.25 (q, 2H, J=7.2 Hz), 3.90 (s, 2H), 1.33 (t, 3H, J=7 Hz).

2-(4-Oxo-3,5-diphenyl-3,4-dihydrothieno[2,3-d]pyrimidin-2-ylsulfanyl)propionic acid methyl ester

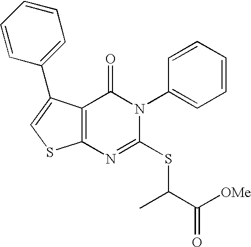

¹H NMR (CDCl₃, 500 MHz) δ (ppm): 7.51 (m, 5H), 7.26-7.35 (m, 5H), 7.01 (s, 1H), 4.51 (q, 1H, J=7.4 Hz), 3.78 (s, 3H), 1.55 (d, 3H, J=7.3 Hz).

(4-Oxo-3,6-diphenyl-3,4-dihydrothieno[2,3-d]pyrimidin-2-ylsulfanyl)acetic acid ethyl ester

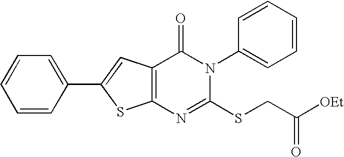

¹H NMR (CDCl₃, 500 MHz) δ (ppm): 7.64 (m, 3H), 7.57 (m, 3H), 7.42 (m, 2H), 7.36 (m, 3H), 4.24 (q, 2H, J=7.2 Hz), 3.87 (s, 2H), 1.33 (t, 3H, J=7.2 Hz).

(6-Methyl-4-oxo-3,5-diphenyl-3,4-dihydrothieno[2,3-d]pyrimidin-2-ylsulfanyl)acetic acid ethyl ester

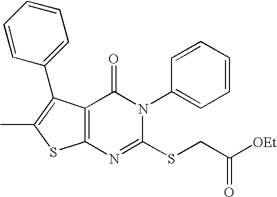

¹H NMR (CDCl₃, 400 MHz) δ (ppm): 7.47 (m, 3H), 7.35 (m, 2H), 7.29 (m, 5H), 4.24 (q, 2H, J=7.1 Hz), 3.86 (s, 2H), 2.35 (s, 3H), 1.33 (t, 3H, J=7.0 Hz).

[6-Chloro-3-(3-fluorophenyl)-5-(2-fluorophenyl)-4-oxo-3,4-dihydrothieno[2,3-d]pyrimidin-2-ylsulfanyl]acetic acid ethyl ester

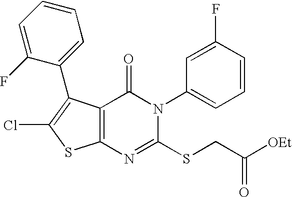

¹H NMR (CDCl₃, 500 MHz) δ (ppm): 7.47 (m, 1H), 7.34 (m, 2H), 7.01-7.22 (m, 5H), 4.25 (q, 2H, J=7 Hz), 3.87 (t, 2H, J=2.5Hz), 1.33 (t, 3H, J=7 Hz).

[3-(3-Fluorophenyl)-5-(2-fluorophenyl)-6-methyl-4-oxo-3,4-dihydrothieno[2,3-d]pyrimidin-2-ylsulfanyl]acetic acid ethyl ester

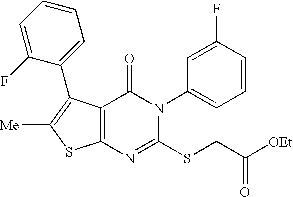

¹H NMR (CDCl₃, 500 MHz) δ (ppm): 7.45 (m, 1H), 7.23-7.32 (m, 2H), 7.02-7.20 (m, 5H), 4.25 (q, 2H, J=7.2 Hz), 3.88 (t, 2H, J=2.7 Hz), 2.34 (s, 3H), 1.33 (t, 3H, J=7.2 Hz).

[3-(3-Fluorophenyl)-5-(2-fluorophenyl)-6-isopropyl-4-oxo-3,4-dihydrothieno[2,3-d]pyrimidin-2-ylsulfanyl]-acetic acid ethyl ester

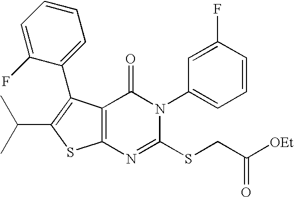

¹H NMR (CDCl₃, 500 MHz) δ (ppm): 7.44 (m, 1H), 7.30 (m, 1H), 6.99-7.25 (m, 6H), 4.25 (q, 2H, J=7.2 Hz), 3.87 (m, 2H), 3.11 (m, 1H), 1.34 (t, 3H, J=7 Hz), 1.29 (d, 3H, J=7 Hz), 1.25 (d, 3H, J=7 Hz).

[3-(3-Fluorophenyl)-4-oxo-3,4,5,6,7,8-hexahydrobenzo[4,5]thieno[2,3-d]pyrimidin-2-ylsulfanyl]acetic acid ethyl ester

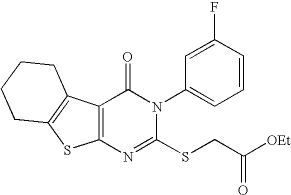

¹H NMR (CDCl₃, 500 MHz) δ (ppm): 7.52 (td, 1H, J=8 Hz, J′=6 Hz), 7.25 (m, 1H), 7.14 (m, 1H), 7.08 (dt, 1H, J=9 Hz, J′=2 Hz), 4.22 (q, 2H, J=7.2 Hz), 3.86 (s, 2H), 2.93 (m, 2H), 2.76 (m, 2H), 1.87 (m, 2H), 1.81 (m, 2H), 1.30 (t, 3H, J=7.2 Hz).

[3-(3-Fluorophenyl)-5-methyl-4-oxo-3,4,5,6,7,8-hexahydrobenzo[4,5]thieno[2,3-d]pyrimidin-2-ylsulfanyl]acetic acid ethyl ester

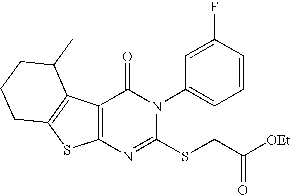

¹H NMR (CDCl₃, 500 MHz) δ (ppm): 7.52 (m, 1H), 7.05-7.26 (m, 3H), 4.22 (q, 2H, J=7.2 Hz), 3.85 (m, 2H), 3.37 (m, 1H), 2.66-2.80 (m, 2H), 1.79-1.98 (m, 3H), 1.71 (m, 1H), 1.31 (t, 3H, J=7 Hz), 1.25 (m, 3H).

[3-(3-Fluorophenyl)-4-oxo-5-phenyl-3,4,5,6,7,8-hexahydrobenzo[4,5]thieno[2,3-d]pyrimidin-2-ylsulfanyl]acetic acid ethyl ester

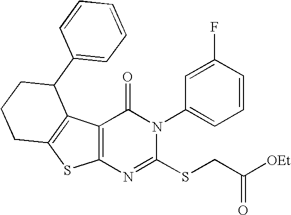

¹H NMR (CDCl₃, 500 MHz) δ (ppm): 7.41 (m, 1H), 7.21 (m, 2H), 7.13 (m, 2H), 6.90-7.05 (m, 4H), 4.68 (bs, 1H), 4.23 (q, 2H, J=7.2 Hz), 3.84 (m, 2H), 2.91 (m, 1H), 2.80 (m, 1H), 2.08 (m, 1H), 1.92 (m, 1H), 1.75 (m, 2H), 1.33 (t, 3H, J=7.5 Hz).

[5-Benzyl-3-(3-fluorophenyl)-4-oxo-3,4,5,6,7,8-hexahydrobenzo[4,5]thieno[2,3-d]pyrimidin-2-ylsulfanyl]acetic acid ethyl ester

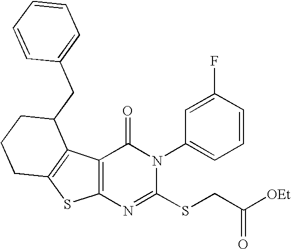

¹H NMR (CDCl₃, 500 MHz) δ (ppm): 7.55 (m, 1H), 7.33 (d, 2H, J=8 Hz), 7.10-7.29 (m, 6H), 4.23 (q, 2H, J=7 Hz), 3.86 (t, 2H, 2.7 Hz), 3.52 (m, 1H), 3.30 (d, 1H, J=12 Hz), 2.83 (m, 1H), 2.72 (m, 1H), 2.48 (m, 1H), 1.99 (m, 1H), 1.84 (m, 1H), 1.71 (d, 1H, J=14 Hz), 1.50 (m, 1H), 1.32 (t, 3H, J=7.2 Hz).

2-Ethoxycarbonylmethylsulfanyl-3-(3-fluorophenyl)-4-oxo-3,5,6,8-tetrahydro-4H-9-thia-1,3,7-triazafluorene-7-carboxylic acid tert-butyl ester

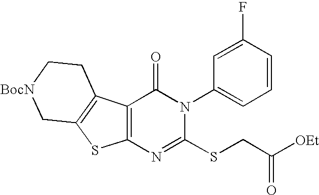

¹H NMR (CDCl₃, 500 MHz) δ (ppm): 7.53 (td, 1 H, J=8 Hz, J′=6 Hz), 7.26 (m, 1H), 7.14 (d, 1H, J=7.5 Hz), 7.08 (dt, 1H, J=8.5 Hz, J′=2 Hz), 4.61 (bs, 2H), 4.22 (q, 2H, J=7.2 Hz), 3.86 (s, 2H), 3.69 (bs, 2H), 3.02 (bs, 2H), 1.49 (s, 9H), 1.31 (t, 3H, J=7.2 Hz).

### Example 9

### Functionalization of the Fused Piperidine Analog

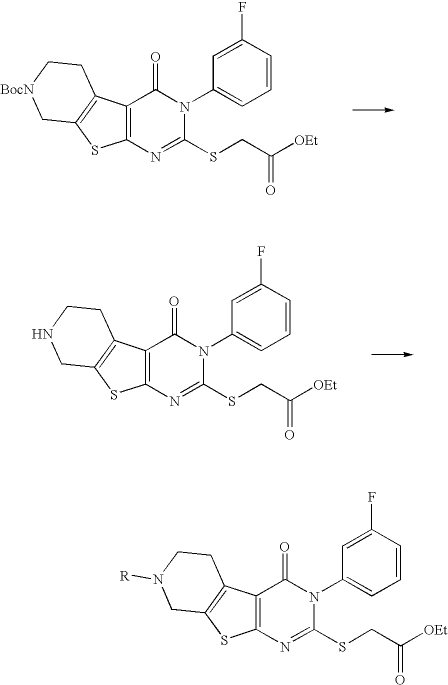

Trifluoroacetic acid (1.5 mL) was added to a solution of 2-ethoxycarbonylmethylsulfanyl-3 -(3 -fluorophenyl)-4-oxo-3,5,6,8-tetrahydro-4H-9-thia-1,3,7-triazafluorene-7-carboxylic acid tert-butyl ester (400 mg) in CH₂Cl₂. The resulting mixture was stirred at room temperature for 2 h and then treated with NaOH 1N and extracted with CH₂Cl₂. The combined organic layers were dried over anhydrous sodium sulfate, filtered and evaporated to afford [3-(3-fluorophenyl)-4-oxo-3,4,5,6,7,8-hexahydro-9-thia-1,3,7-triazafluoren-2-ylsulfanyl]acetic acid ethyl ester (318 mg) as a solid.

[3-(3-Fluorophenyl)-4-oxo-3,4,5,6,7,8-hexahydro-9-thia-1,3,7-triazafluoren-2-ylsulfanyl]acetic acid ethyl ester

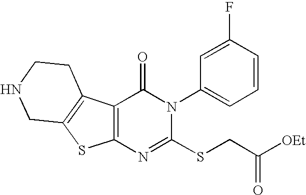

¹H NMR (CDCl₃, 500 MHz) δ (ppm): 7.53 (td, 1H, J=8.5 Hz, J'═δ Hz), 7.26 (m, 1H), 7.14 (d, 1H, J=7.5 Hz), 7.09 (dt, 1H, J=9 Hz, J′=2 Hz), 4.22 (q, 2H, J=7 Hz), 4.03 (bs, 2H), 3.86 (s, 2H), 3.15 (t, 2H, J=6 Hz), 2.96 (m, 2H), 1.30 (t, 3H, J=7 Hz).

At room temperature, to a solution of [3-(3-fluorophenyl)-4-oxo-3,4,5,6,7,8-hexahydro-9-thia- 1,3,7-triazafluoren-2-ylsulfanyl]acetic acid ethyl ester (100 mg, 1 eq.) in CH₂Cl₂ (1 mL) and methanol (0.5 mL) were sequentially added formaldehyde (37% in water, 36 μL, 2 eq.), AcOH (17 μL, 1.2 eq.) and NaBH(OAc)₂ (76 mg, 1.5 Seq.), portion wise. The resulting mixture was stirred at room temperature for 1 h and then treated with NaOH 0.1 N and extracted with CH₂Cl₂. The combined organic layers were dried over anhydrous sodium sulfate, filtered and evaporated to afford [3-(3-fluorophenyl)-7-methyl-4-oxo-3,4,5,6,7,8-hexahydro-9-thia-1,3,7-triazafluoren-2-ylsulfanyl]acetic acid ethyl ester (318 mg) as a solid.

[3-(3-Fluorophenyl)-7-methyl-4-oxo-3,4,5,6,7,8-hexahydro-9-thia-1,3,7-triazafluoren-2-ylsulfanyl]acetic acid ethyl ester

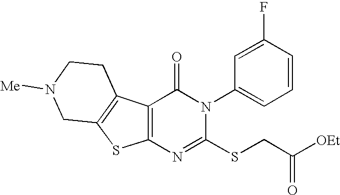

¹H NMR (CDCl₃, 500 MHz) δ (ppm): 7.53 (td, 1H, J=8′=6Hz, J'═δ Hz), 7.25 (m, 1H), 7.15 (d, 1H, J=7.5 Hz), 7.09 (dt, 1H, J=9 Hz, J′=2 Hz), 4.22 (q, 2H, J=7 Hz), 3.86 (s, 2H), 3.63 (bs, 2H), 3.06 (m, 2H), 2.75 (t, 2H, J=5.7 Hz), 2.50 (s, 3H), 1.30 (t, 3H, J=7 Hz).

At room temperature, to a solution of [3-(3-fluorophenyl)-4-oxo-3,4,5,6,7,8-hexahydro-9-thia- 1,3,7-triazafluoren-2-ylsulfanyl]acetic acid ethyl ester (100 mg, 1 eq.) in CH₂Cl₂ (1 mL) and methanol (0.5 mL) were sequentially added propionaldehyde (35 μL, 2 eq.), AcOH (17 μL, 1.2 eq.) and NaBH(OAc)₂ (76 mg, 1.5 eq.), portion wise. The resulting mixture was stirred at room temperature for 16 h and then treated with NaOH 0.1N and extracted with CH₂Cl₂. The combined organic layers were dried over anhydrous sodium sulfate, filtered and evaporated affording [3-(3 -fluorophenyl)-4-oxo-7-propyl-3,4,5,6,7,8-hexahydro-9-thia-1,3,7-triazafluoren-2-ylsulfanyl]acetic acid ethyl ester as a solid.

[3-(3-Fluorophenyl)-4-oxo-7-propyl-3,4,5,6,7,8-hexahydro-9-thia-1,3,7-triazafluoren-2-ylsulfanyl]acetic acid ethyl ester

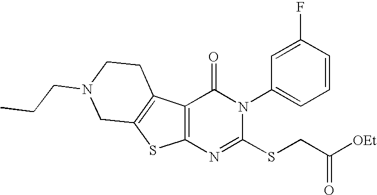

¹H NMR (CDCl₃, 500 MHz) δ (ppm): 7.52 (td, 1H, J=8 Hz, J′=6 Hz), 7.25 (m, 1H), 7.14 (d, 1H, J=7.5 Hz), 7.09 (dt, 1H, J=9 Hz, J′=2 Hz), 4.22 (q, 2H, J=7 Hz), 3.86 (s, 2H), 3.68 (s, 2H), 3.04 (m, 2H), 2.80 (t, 2H, J=5.7 Hz), 2.53 (t, 2H, J=8 Hz), 1.60 (m, 2H), 1.30 (t, 3H, J=7.2 Hz), 0.95 (t, 3H, J=7.2 Hz).

### Example 10

### Functionalization of the 2-methoxyphenvl Analog

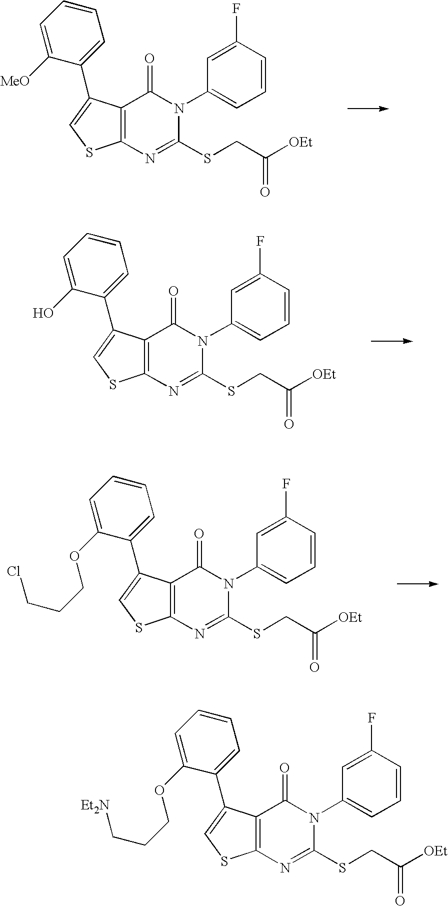

At −78° C., under argon, BBr₃ (1M/CH₂Cl₂, 2.82 mL, 5 eq.) was added to a solution of [3-(3-fluorophenyl)-5-(2-methoxyphenyl)-4-oxo-3,4-dihydrothieno[2,3-d]pyrimidin-2-ylsulfanyl]acetic acid ethyl ester (265 mg, 1 eq.) in CH₂Cl₂ (5.6 mL). The resulting mixture was stirred at room temperature for 1 h and then treated with brine, diluted with water and extracted with CH₂Cl₂. The combined organic layers were dried over anhydrous sodium sulfate, filtered and evaporated. The residue was purified by chromatography on silica gel, eluting with hexane and AcOEt, affording [3-(3-Fluorophenyl)-5-(2-hydroxyphenyl)-4-oxo-3,4-dihydrothieno[2,3-d]pyrimidin -2-ylsulfanyl]acetic acid ethyl ester (223 mg) as a solid.

[3-(3-Fluorophenyl)-5-(2-hydroxyphenyl)-4-oxo-3,4-dihydrothieno[2,3-d]pyrimidin-2-ylsulfanyl]acetic acid ethyl ester

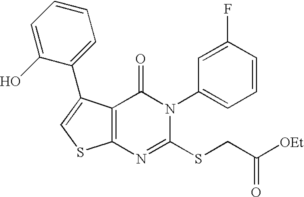

¹H NMR (CDCl₃, 500 MHz) δ (ppm): 7.52 (td, 1H, J=8 Hz, J′=6 Hz), 7.27 (m, 3H), 7.14 (m, 1H), 7.08 (m, 2H), 7.00 (m, 2H), 6.84 (s, 1H), 4.25 (q, 2H, J=7.2 Hz), 3.92 (s, 2H), 1.33 (t, 3H, J=7.2 Hz).

To a solution of [3-(3-fluorophenyl)-5-(2-hydroxyphenyl)-4-oxo-3,4-dihydrothieno[2,3-d]pyrimidin-2-ylsulfanyl]acetic acid ethyl ester (125 mg, 1 eq.) in DMF (1 mL) was added K₂CO₃ (76 mg, 2 eq.) and 1-bromo-3-chloropropane (54 μL, 2 eq.). The mixture was stirred at room temperature in a closed flask for 24 h. Then additional K₂CO₃ (76 mg, 2 eq.) and 1-bromo-3-chloropropane (54 μL, 2 eq.) were added and the mixture was stirred for another 24 h and then treated with saturated NaHCO₃ and extracted with AcOEt. The combined organic layers were washed with brine, dried over anhydrous sodium sulfate, filtered and evaporated. The residue was purified by chromatography on silica gel, eluting with hexane and AcOEt, affording [5 -[2-(3 -chloropropoxy)phenyl]-3 -(3 -fluorophenyl)-4-oxo-3,4-dihydrothieno[2,3-d]pyrimidin-2-ylsulfanyl]acetic acid ethyl ester (102 mg, 70%) as a solid.

[5-[2-(3-Chloropropoxy)phenyl]-3-(3-fluorophenyl)-4-oxo-3,4-dihydrothieno[2,3-d]pyrimidin-2-ylsulfanyl]acetic acid ethyl ester

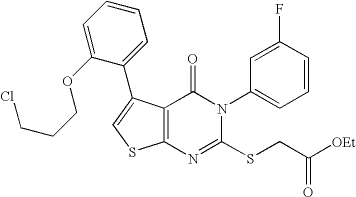

¹H NMR (CDCl₃, 500 MHz) δ (ppm): 7.47 (td, 1H, J=8 Hz, J′=6 Hz), 7.27 (m, 2H), 7.20 (td, 1H, J=9 Hz, J′=2 Hz), 7.11 (m, 1H), 7.05 (dt, 1H, J=8.5 Hz, J′=2 Hz), 6.99 (s, 1H), 6.95 (t, 1H, J=7.7 Hz), 6.89 (d, 1H, J=8 Hz), 4.25 (q, 2H, J=7.2 Hz), 4.05 (t, 2H, J=6 Hz), 3.91 (s, 2H), 3.49 (t, 2H, J=6.7 Hz), 2.07 (m, 2H), 1.33 (t, 3H, J=7.2 Hz).

A mixture of [5-[2-(3-chloropropoxy)phenyl]-3-(3-fluorophenyl)-4-oxo-3,4-dihydrothieno[2,3-d]pyrimidin-2-ylsulfanyl]acetic acid ethyl ester (50 mg, 1 eq.), KI (16 mg, 1 eq.), K₂CO₃ (13 mg, 1 eq.) and Et₂NH (156 mg, 16 eq.) in MeCN (1 mL) was stirred for 48 h at 80° C. in a closed flask. The reaction mixture was treated with water and extracted with CH₂Cl₂. The combined organic layers were dried over anhydrous sodium sulfate, filtered and evaporated. The residue was purified by chromatography on silica gel, eluting with CH₂Cl₂ and EtOH, affording [5-[2-(3-Diethylaminopropoxy)phenyl]-3 -(3 -fluoro-phenyl)-4-oxo-3,4-dihydrothieno[2,3 -d]pyrimidin-2-ylsulfanyl]acetic acid ethyl ester (47 mg).

[5-[2-(3-Diethylaminopropoxy)phenyl]-3-(3-fluorophenyl)-4-oxo-3,4-dihydrothieno[2,3-d]pyrimidin-2-ylsulfanyl]acetic acid ethyl ester

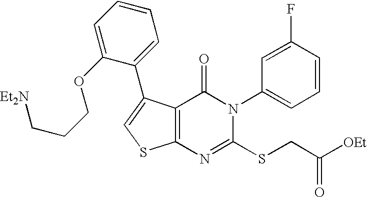

¹H NMR (CDCl₃, 500 MHz) δ (ppm): 7.46 (td, 1H, J=8 Hz, J′=6 Hz), 7.27 (m, 2H), 7.19 (td, 1H, J=7.5 Hz, J′=2.5 Hz), 7.13 (m, 1H), 7.09 (dt, 1H, J=9 Hz, J′=2 Hz), 6.99 (s, 1H), 6.92 (t, 1H, J=7 Hz), 6.86 (d, 1H, J=8.5 Hz), 4.25 (q, 2H, J=7.2 Hz), 3.93 (t, 2H, J=7 Hz), 3.89 (d, 2H, J=2 Hz), 2.47 (bs, 6H), 1.75 (bs, 2H),1.33 (t, 3H, J=7 Hz), 0.96 (t, 6H, J=7 Hz).

### Example 11

### Aminolysis of the Ester

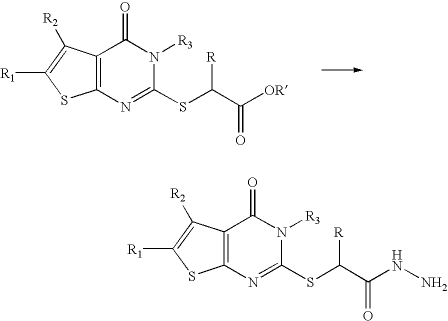

The methyl or ethyl ester was suspended in ethanol (0.4 M), and hydrazine was added (15 eq.). The reaction mixture was stirred at room temperature until completion (1-12 h). In case were the starting material was too insoluble to react, THF (0.8 M) was added. After the reaction was complete, the product was either filtered from the reaction mixture and rinsed with ethanol, or isolated after silica gel chromatography.

(4-Oxo-3,5-diphenyl-3,4-dihydro-thieno[2,3-d]pyrimidin-2-ylsulfanyl)acetic acid hydrazide ( 986-01)

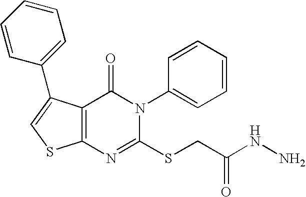

¹H NMR (CDCl₃, 500 MHz) δ (ppm): 7.84 (bs, 1H), 7.52 (m, 5H), 7.27-7.34 (m, 5H), 7.07 (s, 1H), 3.92 (bs, 2H), 3.78 (s, 2H).

(Quinolin-2-ylsulfanyl)acetic acid hydrazide (986-02)

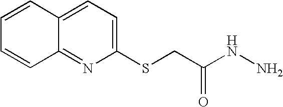

¹H NMR (CDCl₃, 500 MHz) δ (ppm): 8.84 (bs, 1H), 7.98 (d, 1H, J=9 Hz), 7.96 (d, 1H, J=8 Hz), 7.77 (dd, 1H, J=8 Hz, J′=1.5 Hz), 7.71 (ddd, 1H, J=8 Hz, J′=7 Hz, J″=15 Hz), 7.50 (ddd, 1H, J=8 Hz, J′=7 Hz, J″=1 Hz), 7.27 (d, 1H, J=8 Hz), 3.96 (s, 2H), 3.87 (bs, 2H).

(4-Oxo-3-phenyl-3,4-dihydroquinazolin-2-ylsulfanyl)acetic acid hydrazide (986-03)

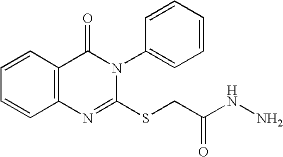

¹H NMR (CDCl₃, 400 MHz) δ (ppm): 8.31 (bs, 1H), 8.27 (dd, 1H, J=8 Hz), 7.79 (t, 1H, J=7.7 Hz), 7.64 (d, 1H, J=8 Hz), 7.57 (m, 3H), 7.47 (t, 1H, J=7.7 Hz), 7.33 (m, 2H), 3.89 (bs, 2H), 3.78 (s, 2H).

[3-(3-Fluorophenyl)-4-oxo-6-phenyl-3,4-dihydroquinazolin-2-ylsulfanyl]acetic acid hydrazide (986-04)

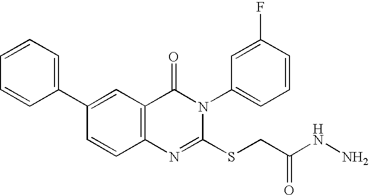

¹H NMR (CDCl₃, 500 MHz) δ (ppm): 8.47 (d, 1H, J=2 Hz), 8.22 (bs, 1H), 8.04 (dd, 1H, J=8 Hz, J′=2 Hz), 7.69 (m, 3H), 7.55 (td, 1H, J=8 Hz, J′=6 Hz), 7.49 (m, 2H), 7.41 (t, 1H, J=7 Hz), 7.29 (td, 1H, J=8.5 Hz, J′=2.5 Hz), 7.16 (d, 1H, 8 Hz), 7.10 (dt, 1H, J=8 Hz, J′=2 Hz), 3.92 (bs, 2H), 3.84 (d, 1H, J=15 Hz), 3.79 (d, 1H, J=15 Hz).

(4-Oxo-3,5-diphenyl-3,4-dihydrothieno[2,3-d]pyrimidin-2-ylamino)acetic acid hydrazide (986-05)

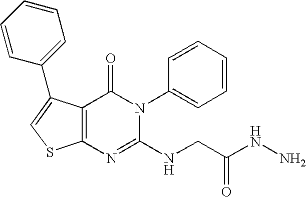

¹H NMR (CDCl₃, 500 MHz) δ (ppm): 7.50-7.59 (m, 5H), 7.37 (bs, 1H), 7.27-7.34 (m, 5H), 6.78 (s, 1H), 4.89 (t, 1H, J=5 Hz), 4.02 (d, 2H, J=5 Hz), 3.86 (bs, 2H).

[Methyl-(4-oxo-3,5-diphenyl-3,4-dihydrothieno[2,3-d]pyrimidin-2-yl) -amino]acetic acid hydrazide (986-06)

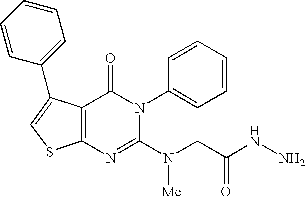

¹H NMR (CDCl₃, 500 MHz) δ (ppm): 11.89 (s, 1H), 7.93 (bs, 1H), 7.53 (m, 3H), 7.49 (m, 2H), 7.21 (m, 3H), 7.03 (m, 3H), 6.50 (s, 1H), 3.87 (s, 2H), 3.13 (s, 3H).

[3-(4-Fluorophenyl)-4-oxo-5-phenyl-3,4-dihydrothieno[2,3-d]pyrimidin-2-ylsulfanyl]acetic acid hydrazide (986-07)

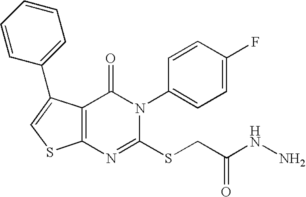

¹H NMR (DMSO-d₆, 500 MHz) δ (ppm): 9.32 (bs, 1H), 7.52 (m, 2H), 7.49 (m, 2H), 7.45 (s, 1H), 7.41 (t, 2H, J=8.8Hz), 7.33 (m, 3H), 4.27 (bm, 2H), 3.85 (s, 2H).

[5-(4-Fluorophenyl)-4-oxo-3-phenyl-3,4-dihydrothieno[2,3-d]pyrimidin-2-ylsulfanyl]acetic acid hydrazide (986-08)

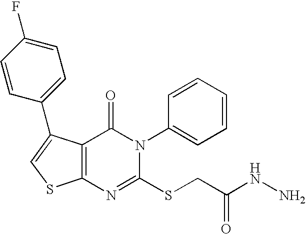

¹H NMR (CDCl₃, 500 MHz) δ (ppm): 7.80 (bs, 1H), 7.47-7.54 (m, 5H), 7.29 (m, 2H), 7.03 (m, 3H), 3.92 (bs, 2H), 3.78 (s, 2H).

[3,5-Bis-(4-fluorophenyl)-4-oxo-3,4-dihydrothieno[2,3-d]pyrimidin-2-ylsulfanyl]acetic acid hydrazide (986-09)

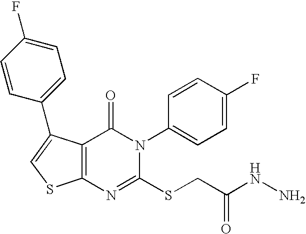

¹H NMR (DMSO-d₆, 500 MHz) δ (ppm): 9.32 (bs, 1H), 7.52 (m, 4H), 7.46 (s, 1H), 7.41 (t, 2H, J=8.9 Hz), 7.18 (t, 2H, J=9.2 Hz), 4.27 (bd, 2H), 3.85 (s, 2H).

(3-Methyl-4-oxo-5-phenyl-3,4-dihydrothieno[2,3-d]pyrimidin-2-ylsulfanyl)acetic acid hydrazide (986-10)

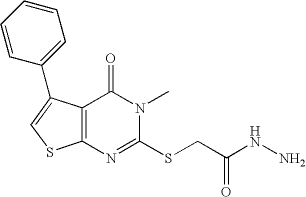

¹H NMR (CDCl₃, 500 MHz) δ (ppm): 7.85 (bs, 1H), 7.51 (m, 2H), 7.36-7.43 (m, 3H), 7.01 (s, 1H), 3.93 (s, 4H), 3.57 (s, 3H).

[3-(4-Fluorophenyl)-5-methyl-4-oxo-3,4-dihydrothieno[2,3-d]pyrimidin-2-ylsulfanyl]acetic acid hydrazide (986-11)

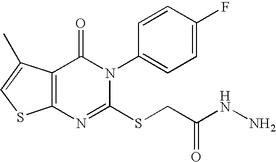

¹H NMR (CDCl₃, 500 MHz) δ (ppm): 7.79 (bs, 1H), 7.22-7.30 (m, 4H), 6.75 m, 1H), 3.90 (bs, 2H), 3.75 (s, 2H), 2.52 (d, 3H, J=1.5 Hz).

2-(4-Oxo-3,5-diphenyl-3,4-dihydrothieno[2,3-d]pyrimidin-2-ylsulfanyl)propionic acid hydrazide (986-12)

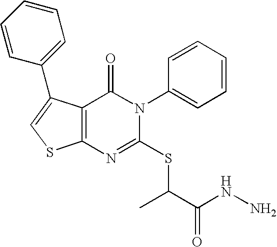

1H NMR (CDCl₃, 500 MHz) δ (ppm): 7.94 (bs, 1H), 7.51 (m, 5H), 7.33 (m, 3H), 7.26 (m, 2H), 7.05 (s, 1H), 4.43 (q, 1H, J=7.3 Hz), 1.49 (d, 3H, J=7 Hz).

(6-Methyl-4-oxo-3,5-diphenyl-3,4-dihydrothieno[2,3-d]pyrimidin-2-ylsulfanyl)acetic acid hydrazide (986-13)

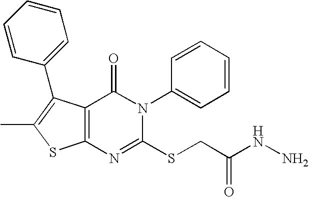

¹H NMR (CDCl₃, 400MHz) δ (ppm): 7.90 (bs, 1H), 7.48 (m, 3H), 7.36 (t, 2H, J=7.3 Hz), 7.30 (m, 3H), 7.24 (m, 2H), 3.92 (bs, 2H), 3.76 (s, 2H), 2.37 (s, 3H).

(3-Benzyl-4-oxo-5-phenyl-3,4-dihydrothieno[2,3-d]pyrimidin-2-ylsulfanyl)acetic acid hydrazide (986-14)

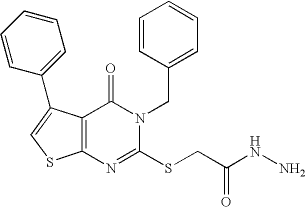

¹H NMR (CDCl₃, 400 MHz) δ (ppm): 7.76 (bs, 1H), 7.55 (d, 2H, J=6.6 Hz), 7.41 (m, 3H), 7.29 (m, 5H), 7.04 (s, 1H), 5.37 (s, 2H), 3.89 (s, 2H), 3.87 (s, 2H).

[3-(4-Methoxyphenyl)-4-oxo-5-phenyl-3,4-dihydrothieno[2,3-d]pyrimidin -2-ylsulfanyl]acetic acid hydrazide (986-15)

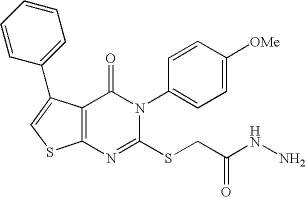

¹H NMR (CDCl₃, 400 MHz) δ (ppm): 7.86 (bs,1 H), 7.53 (d, 2H, J=6.6 Hz), 7.34 (m, 3H), 7.19 (d, 2H, J=9.1 Hz), 7.06 (s, 1H), 7.01 (d, 2H, J=8.8 Hz), 3.92 (bm, 2H), 3.85 (s, 3H), 3.77 (s, 2H).

(4-Oxo-5-phenyl-3-pyridin-2-yl-3,4-dihydrothieno[2,3-d]pyrimidin-2-ylsulfanyl)acetic acid hydrazide (986-16)

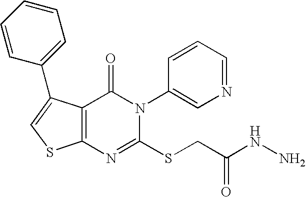

¹H NMR (CDCl₃, 400 MHz) δ (ppm): 8.75 (dd, 1H, J=4.8 Hz, J′=1.5 Hz), 8.57 (d, 1H, J=2.2 Hz), 7.75 (bs, 1H), 7.67 (d, 1H, J=8.0 Hz), 7.50 (m, 3H), 7.36 (m, 3H), 7.10 (s, 1H), 3.93 (bm, 2H), 3.86 (d, 1H, J=14.6 Hz), 3.77 (d, 1H, J=14.6 Hz).

(3-Cyclohexyl-4-oxo-5-phenyl-3,4-dihydrothieno[2,3-d]pyrimidin-2-ylsulfanyl)acetic acid hydrazide (986-17)

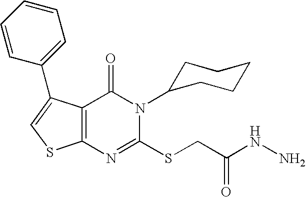

¹H NMR (CDCl₃, 500 MHz) δ (ppm): 7.83 (bs, 1H), 7.49 (m, 2H), 7.35-7.43 (m, 3H), 6.95 (s, 1H), 4.09 (m, 1H), 3.92 (m, 2H), 3.89 (s, 2H), 2.71 (m, 2H), 1.87 (d, 2, J=13.5 Hz), 1.73 (d, 2H, J=11 Hz), 1.64 (d, 1H, J=10 Hz), 1.30 (m, 3H).

[3-(2-Fluorophenyl)-4-oxo-5-phenyl-3,4-dihydrothieno[2,3-d]pyrimidin-2-ylsulfanyl]acetic acid hydrazide (986-18)

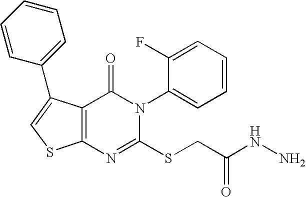

¹H NMR (CDCl₃, 500 MHz) δ (ppm): 7.81 (bs, 1H), 7.52 (m, 3H), 7.24-7.38 (m, 6H), 7.08 (s, 1H), 3.92 (bs, 2H), 3.81 (q, 2H, J=15 Hz).

[3-(3-Fluorophenyl)-4-oxo-5-phenyl-3,4-dihydrothieno[2,3-d]pyrimidin-2-ylsulfanyl]acetic acid hydrazide (986-19)

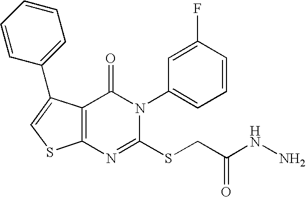

¹H NMR (CDCl₃, 500 MHz) δ (ppm): 7.77 (bs, 1H), 7.50 (m, 3H), 7.34 (m, 3H), 7.23 (td, 1H, J=8.2 Hz, J′=2.5 Hz), 7.10 (d. 1H, 7 Hz), 7.08 (s, 1H), 7.04 (dt, 1H, J=9 Hz, J′=2 Hz), 3.93 (bs, 2H), 3.81 (d, 1H, J=14.5 Hz), 3.77 (d, 1H, J=14.5 Hz).

[3-(2-Methoxyphenyl)-4-oxo-5-phenyl-3,4-dihydrothieno[2,3-d]pyrimidin-2-ylsulfanyl]acetic acid hydrazide (986-20)

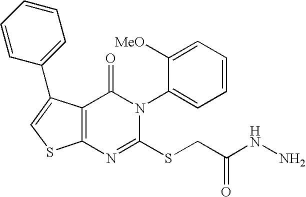

¹H NMR (CDCl₃, 500 MHz) δ (ppm): 7.94 (bs, 1H), 7.54 (m, 2H), 7.48 (td, 1H, J=8 Hz, J′=1.5 Hz), 7.29-7.36 (m, 3H), 7.21 (dd, 1H, J=8 Hz, J′=1.5 Hz), 7.03-7.09 (m, 2H), 7.05 (s, 1H), 3.90 (bs, 2H), 3.74-3.83 (m, 5H).

[3-(3-Methoxyphenyl)-4-oxo-5-phenyl-3,4-dihydrothieno[2,3-d]pyrimidin-2-ylsulfanyl]acetic acid hydrazide (986-21)

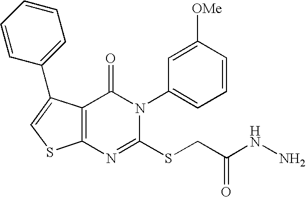

¹H NMR (CDCl₃, 500 MHz) δ (ppm): 7.85 (bs, 1H), 7.53 (m, 2H), 7.42 (t, 1H, J=8.5 Hz), 7.34 (m, 3H), 7.06 (s, 1H), 7.04 (m, 1H), 6.87 (m, 1H), 6.80 (t, 1H, J=4 Hz), 3.92 (bs, 2H), 3.81 (s, 3H), 3.78 (s, 2H).

3-(6-Methyl-4-oxo-3,5-diphenyl-3,4-dihydrothieno[2,3-d]pyrimidin-2-ylsulfanyl)propionic acid hydrazide (986-22)

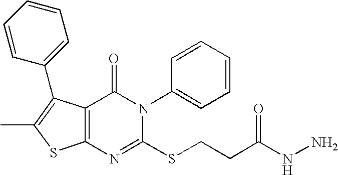

¹H NMR (CDCl₃, 500 MHz) δ (ppm): 7.45 (m, 3H), 7.35 (m, 2H), 7.29 (m, 3H), 7.21 (m, 2H), 6.85 (bs, 1H), 3.89 (bs, 2H), 3.39 (t, 2H, J=7 Hz), 2.62 (t, 2H, J=7 Hz), 2.36 (s, 3H).

[3-(2-Chlorophenyl)-4-oxo-5-phenyl-3,4-dihydrothieno[2,3-d]pyrimidin-2-ylsulfanyl]acetic acid hydrazide (986-23)

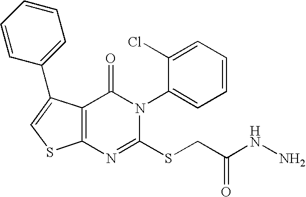

¹H NMR (CDCl₃, 500 MHz) δ (ppm): 7.81 (bs, 1H), 7.58 (dd, 1H, J=8.5 Hz, J′=1.5 Hz), 7.53 (m, 2H), 7.48 (td, 1H, J=7.7 Hz, J′=2 Hz), 7.43 (td, 1H, J=7.5 Hz, J′=1.5 Hz), 7.34 (m, 4H), 7.09 (s, 1H), 3.92 (bs, 2H), 3.82 (d, 2H, J=1.5 Hz).

[3-(3-Chlorophenyl)-4-oxo-5-phenyl-3,4-dihydrothieno[2,3-d]pyrimidin-2-ylsulfanyl]acetic acid hydrazide (986-24)

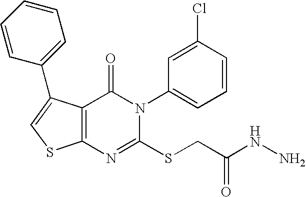

¹H NMR (CDCl₃, 500 MHz) δ (ppm): 7.77 (bs, 1H), 7.50 (m, 3H), 7.46 (t, 1H, J=8 Hz), 7.34 (m, 4H), 7.20 (m, 1H), 7.08 (s, 1H), 3.93 (bs, 2H), 3.80 (d, 2H, J=4 Hz).

[3-(4-Chlorophenyl)-4-oxo-5-phenyl-3,4-dihydrothieno[2,3-d]pyrimidin-2-ylsulfanyl]acetic acid hydrazide (986-25)

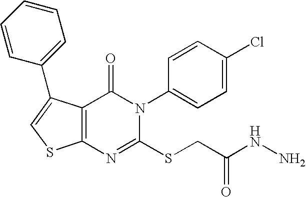

¹H NMR (CDCl₃, 500 MHz) δ (ppm): 7.77 (bs, 1H), 7.50 (m, 4H), 7.33 (m, 3H), 7.23 (d, 2H, J=8 Hz), 7.08 (s, 1H), 3.92 (bs, 2H), 3.79 (s, 2H).

[3-(2,5-Difluorophenyl)-4-oxo-5-phenyl-3,4-dihydrothieno[2,3-d]pyrimidin-2-ylsulfanyl]acetic acid hydrazide (986-26)

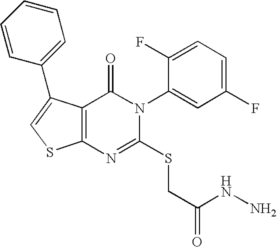

¹H NMR (CDCl₃, 500 MHz) δ (ppm): 7.75 (bs, 1H), 7.51 (m, 2H), 7.35 (m, 3H), 7.23 (m, 2H), 7.09 (s, 1H), 7.06 (m, 1H), 3.92 (m, 2H), 3.85 (d, 1H, J=15 Hz), 3.81 (d, 1H, J=14.5 Hz).

[3-(3,5-Difluorophenyl)-4-oxo-5-phenyl-3,4-dihydrothieno[2,3-d]pyrimidin-2-ylsulfanyl]acetic acid hydrazide (986-27)

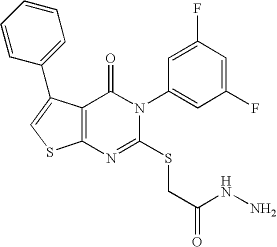

¹H NMR (CDCl₃, 500 MHz) δ (ppm): 7.71 (bs, 1H), 7.50 (d, 2H, J=7 Hz), 7.36 (m, 3H), 7.09 (s, 1H), 6.99 (tt, 1H, J=8.7 Hz, J′=2 Hz), 6.88 (dd, 2H, J=6 Hz, J′=2 Hz), 3.94 (bs, 1H), 3.81 (s, 2H).

[3-(2,6-Difluorophenyl)-4-oxo-5-phenyl-3,4-dihydrothieno[2,3-d]pyrimidin-2-ylsulfanyl]acetic acid hydrazide (986-28)

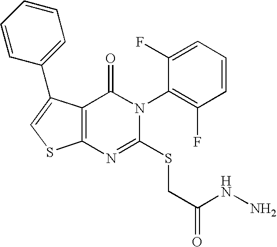

¹H NMR (CDCl₃, 500 MHz) δ (ppm): 7.78 (bs, 1H), 7.51 (m, 3H), 7.35 (m, 3H), 7.10 (s, 1H), 7.09 (m, 2H), 3.93 (bs, 2H), 3.85 (s, 2H).

(4-Oxo-3,6-diphenyl-3,4-dihydrothieno[2,3-d]pyrimidin-2-ylsulfanyl)acetic acid hydrazide (986-29)

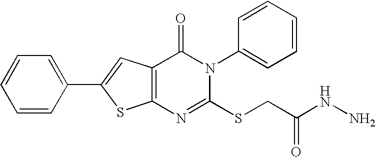

¹H NMR (CDCl₃, 500 MHz) δ (ppm): 7.82 (bs, 1H), 7.65 (m, 3H), 7.57 (m, 3H), 7.44 (t, 2H, J=7.5 Hz), 7.36 (t, 1H, J=7.2 Hz), 7.32 (m, 2H), 3.92 (bs, 2H),3.77 (s, 2H).

(4-Oxo-3,5-diphenyl-3,4-dihydrothieno[2,3-d]pyrimidin-2-yloxy)acetic acid hydrazide (986-30)

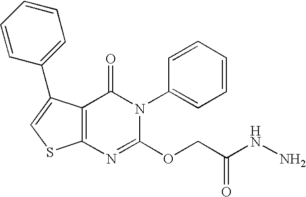

¹H NMR (CDCl₃, 500 MHz) δ (ppm): 7.46-7.56 (m, 5H), 7.26-7.36 (m, 5H), 6.99 (s, 1H), 6.68 (bs, 1H), 4.92 (s, 2H), 3.86 (bs, 2H).

[3-(3-Fluorophenyl)-5-(2-methoxyphenyl)-4-oxo-3,4-dihydrothieno[2,3-d]pyrimidin-2-ylsulfanyl]acetic acid hydrazide (986-31)

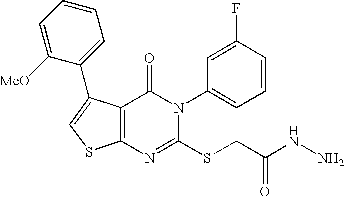

¹H NMR (CDCl₃, 500 MHz) (ppm): 7.84 (bs, 1H), 7.47 (td, 1H, J=8 Hz, J′=6 Hz), 7.30 (t, 1H, J=7.7 Hz), 7.26 (m, 1H), 7.21 (t, 1H, J=8.5 Hz), 7.06 (d, 1H, J=8 Hz), 7.05 (s, 1H), 7.00 (dt, 1H, J=9 Hz, J′=2 Hz), 6.95 (t, 1H, J=7.7 Hz), 6.90 (d, 1H, J=8.5 Hz), 3.94 (bs, 2H), 3.80 (d, 1H, J=14.5 Hz), 3.77 (d, 1H, J=14.5 Hz), 3.74 (s, 3H).

[3-(3-Fluorophenyl)-5-(3-methoxyphenyl)-4-oxo-3,4-dihydrothieno[2,3-d]pyrimidin-2-ylsulfanyl]acetic acid hydrazide (986-32)

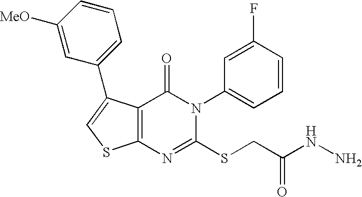

¹H NMR (DMSO-d₆, 500 MHz) δ (ppm): 9.32 (bs, 1H), 7.62 (m, 1H), 7.48 (m, 2H), 7.43 (td, 1H, J=7.7 Hz, J′=2 Hz), 7.34 (d, 1H, J=8 Hz), 7.26 (t, 1H, J=7.7 Hz), 7.06 (m, 2H), 6.89 (m, 1H), 4.28 (bm, 2H), 3.86 (s, 2H), 3.74 (s, 3H).

[3-(3-Fluorophenyl)-5-(4-methoxyphenyl)-4-oxo-3,4-dihydrothieno[2,3-d]pyrimidin-2-ylsulfanyl]acetic acid hydrazide (986-33)

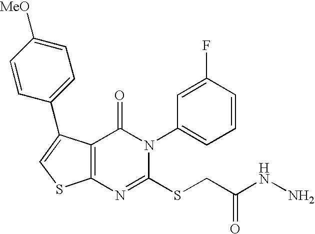

¹H NMR (CDCl₃ 500 MHz) δ (ppm): 7.79 (bs, 1H), 7.50 (td, 1H, J=8 Hz, J′=6 Hz), 7.45 (d, 2H, J=9 Hz), 7.23 (td, 1H, J=8.7 Hz, J′=2.5 Hz), 7.10 (d, 1H, J=7.5 Hz), 7.04 (dt, 1H, J=9 Hz, J′=2.5 Hz), 7.02 (s, 1H), 6.89 (d, 2H, J=8.5 Hz), 3.93 (bs, 2H), 3.79 (m, 5H).

[3-(3-Fluorophenyl)-5-(2-hydroxyphenyl)-4-oxo-3,4-dihydrothieno[2,3-d]pyrimidin-2-ylsulfanyl]acetic acid hydrazide (986-34)

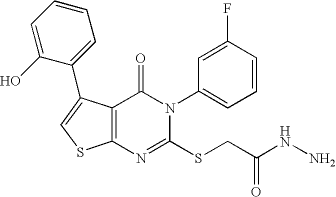

¹H NMR (DMSO-d₆, 500 MHz) δ (ppm): 9.32 (s, 2H), 7.59 (m, 1H), 7.41 (m, 2H), 7.29 (s, 1H), 7.27 (m, 1H), 7.11 (m, 2H), 6.79 (d, 1H, J=8 Hz), 6.74 (t, 1H, J=7.2 Hz), 4.27 (bm, 2H), 3.85 (s, 2H).

[5-[2-(3-Diethylaminopropoxy)phenyl]-3-(3-fluorophenyl)-4-oxo-3,4-dihydrothieno[2,3-d]pyrimidin-2-ylsulfanyl]acetic acid hydrazide (986-35)

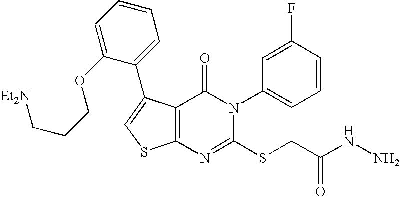

¹H NMR (CDCl₃, 500 MHz) δ (ppm): 7.98 (bs, 1H), 7.46 (td, 1H, J=8 Hz, J′=6 Hz), 7.27 (m, 2H), 7.20 (ts, 1H, J=9 Hz, J′=2.5 Hz), 7.13 (m, 2H), 7.03 (s, 1H), 6.94 (t, 1H, J=7.5 Hz), 6.86 (d, 1H, J=8.5 Hz), 3.94 (t, 2H, J=6 Hz), 3.81 (d, 1H, J=14.5 Hz), 3.77 (d, 1H, J=15 Hz), 2.50 (bs, 6H), 1.13 (m, 2H), 0.98 (t, 6H, J=7 Hz).

[3-(3-Fluorophenyl)-5-isopropyl-4-oxo-3,4-dihydrothieno[2,3-d]pyrimidin-2-ylsulfanyl]acetic acid hydrazide (986-36)

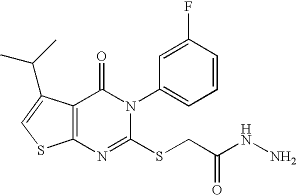

¹H NMR (CDCl₃, 500 MHz) δ (ppm): 7.81 (bs, 1H), 7.53 (td, 1H, J=8 Hz, J′=6 Hz), 7.27 (td, 1H, J=8.2 Hz, J′=2.5 Hz), 7.12 (d, 1H, J=7.5 Hz), 7.06 (dt, 1H, J=8 Hz, J′=2 Hz), 6.84 (d, 1H, J=1 Hz), 3.78 (d, 1H, J=15 Hz), 3.74 (d, 1H, J=15 Hz), 3.62 (m, 1H), 1.27 (d, 6H, J=7 Hz).

[3-(3-Fluorophenyl)-5-(2-fluorophenyl)-4-oxo-3,4-dihydrothieno[2,3-d]pyrimidin-2-ylsulfanyl]acetic acid hydrazide (986-37)

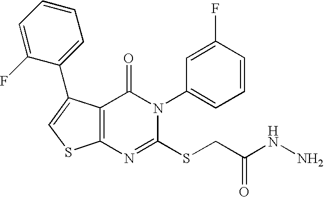

¹H NMR (CDCl₃ 500 MHz) δ (ppm): 7.78 (bs, 1H), 7.48 (td, 1H, J=8.2 Hz, J′=6 Hz), 7.37 (td, 1H, J=7.5 Hz, J′=1.5 Hz), 7.31 (m, 1H), 7.22 (td, 1H, J=8 Hz, J′=2.5 Hz), 7.14 (m, 2H), 7.09 (m, 2H), 7.04 (dt, 1H, J=9 Hz, J′=2 Hz), 3.93 (bs, 2H), 3.81 (d, 1H, J=15 Hz), 3.77 (d, 1H, J=15 Hz).

[3,5-Bis-(3-fluorophenyl)-4-oxo-3,4-dihydrothieno[2,3-d]pyrimidin-2-ylsulfanyl]acetic acid hydrazide (986-38)

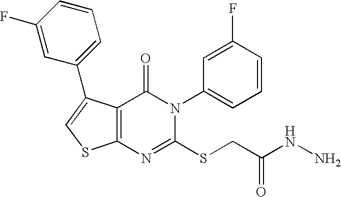

¹H NMR (CDCl₃ 500 MHz) δ (ppm): 7.75 (bs, 1H), 7.51 (td, 1H, J=8.2 Hz, J′=6 Hz), 7.31 (m, 2H), 7.23 (m, 2H), 7.10 (m, 2H), 7.03 (m, 2H), 3.82 (d, 1H, J=14,5 Hz), 3.78 (d, 1H, J=14.5 Hz).

[6-Chloro-3-(3-fluorophenyl)-5-(2-fluorophenyl)-4-oxo-3,4-dihydrothieno[2,3-d]pyrimidin-2-ylsulfanyl]acetic acid hydrazide (986-39)

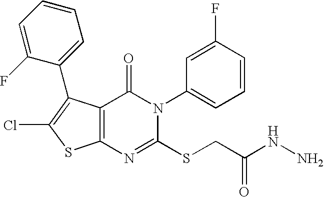

¹H NMR (CDCl₃ 500 MHz) δ (ppm): 7.61 (bs, 1H), 7.48 (m, 1H), 7.35 (m, 2H), 7.19 (m, 2H), 6.97-7.14 (m, 3H), 3.78 (m, 2H).

[3-(3-Fluorophenyl)-5-(2-fluorophenyl)-6-methyl-4-oxo-3,4-dihydrothieno[2,3-d]pyrimidin-2-ylsulfanyl]acetic acid hydrazide (986-40)

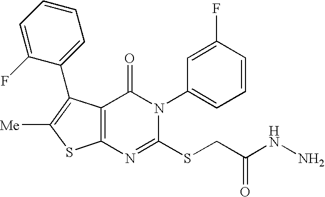

¹H NMR (CDCl₃ 500 MHz) δ (ppm): 7.82 (bs, 1H), 7.46 (m, 1H), 7.31 (m, 1H), 6.98-7.26 (m, 6H), 3.92 (bs, 2H), 3.79 (d, 1H, J=15.5 Hz), 3.75 (d, 1H, J=15.5 Hz), 2.36 (s, 3H).

[3-(3-Fluorophenyl)-5-(2-fluorophenyl)-6-isopropyl-4-oxo-3,4-dihydrothieno[2,3-d]pyrimidin-2-ylsulfanyl]acetic acid hydrazide (986-41)

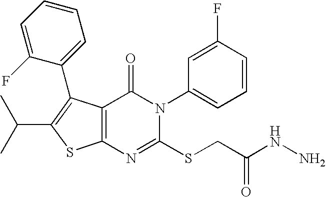

¹H NMR (CDCl₃ 500 MHz) δ (ppm): 7.88 (bs, 1H), 7.45 (m, 1H), 7.32 (m, 1H), 6.96-7.26 (m, 6H), 3.78 (dd, 1H, J=14.5 Hz, J′=1.5 Hz), 3.75 (dd, 1H, J=15 Hz, J′=1 Hz), 3.13 (m, 1H), 1.31 (d, 3H, J=7 Hz), 1.27 (d, 3H, J=6 Hz).

[3-(3-Fluorophenyl)-4-oxo-3,4,5,6,7,8-hexahydrobenzo[4,5]thieno[2,3-d]pyrimidin-2-ylsulfanyl]acetic acid hydrazide (986-42)

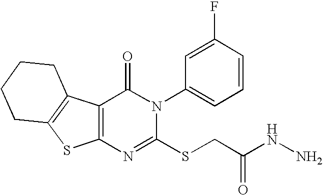

¹H NMR (CDCl₃ 500 MHz) δ (ppm): 7.82 (bs, 1H), 7.52 (td, 1H, J=8 Hz, J′=6 Hz), 7.26 (m, 1H), 7.10 (d, 1H, J=8 Hz), 7.04 (dt, 1H, J=8 Hz, J′=2 Hz), 3.89 (bs, 2H), 3.76 (d, 1H, J=14.5 Hz), 3.72 (d, 1H, J=14.5 Hz), 2.93 (m, 2H), 2.78 (m, 2H), 1.88 (m, 2H), 1.82 (m, 2H).

[3-(3-Fluorophenyl)-5-methyl-4-oxo-3,4,5,6,7,8-hexahydrobenzo[4,5]thieno[2,3-d]pyrimidin-2-ylsulfanyl]acetic acid hydrazide (986-43)

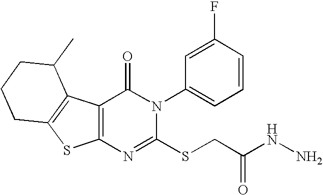

¹H NMR (CDCl₃ 500 MHz) δ (ppm): 7.83 (bs, 1H), 7.53 (m, 1H), 7.26 (m, 1H), 7.02-7.14 (m, 2H), 3.74 (m, 2H), 3.37 (m, 1H), 2.68-2.82 (m, 2H), 1.80-1.97 (m, 3H). 1.72 (m, 1H), 1.25 (dd, 3H, J=7 Hz, J'═2.5 Hz).

[3-(3-Fluorophenyl)-4-oxo-5-phenyl-3,4,5,6,7,8-hexahydrobenzo[4,5]thieno[2,3-d]pyrimidin-2-ylsulfanyl]acetic acid hydrazide (986-44)

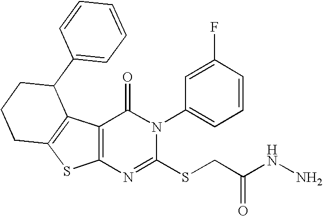

¹H NMR (CDCl₃ 500 MHz) δ (ppm): 7.83 (bs, 1H), 7.42 (m, 1H), 7.22 (m, 2H), 7.15 (m, 2H), 6.86-7.02 (m, 4H), 4.69 (s, 1H), 3.91 (bs, 2H), 3.73 (m, 2H), 2.94 (m, 1H), 2.82 (m, 1H), 2.09 (m, 1H), 1.93 (m, 1H), 1.76 (m, 2H).

[5-Benzyl-3-(3-fluorophenyl)-4-oxo-3,4,5,6,7,8-hexahydrobenzo[4,5]thieno[2,3-d]pyrimidin-2-ylsulfanyl]acetic acid hydrazide (986-45)

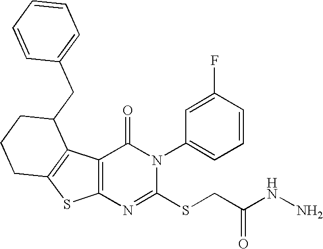

¹H NMR (CDCl₃ 500 MHz) δ (ppm): 7.84 (bs, 1H), 7.55 (m, 1H), 7.32 (d, 2H, J=8 Hz), 7.23-7.30 (m, 3H), 7.06-7.18 (m, 3H), 3.75 (m, 2H), 3.52 (m, 1H), 3.29 (d, 1H, J=13 Hz), 2.86 (dd, 1H, J=17 Hz, J′=5 Hz), 2.74 (m, 1H), 2.49 (m, 1H), 1.99 (m, 1H), 1.86 (m, 1H), 1.72 (d, 1H, J=13.5 Hz), 1.51 (m, 1H).

3-(3-Fluorophenyl)-2-hydrazinocarbonylmethylsulfanyl-4-oxo-3,5,6,8-tetrahydro-4H-9-thia-1,3,7-triazafluorene-7-carboxylic acid tert-butyl ester (986-46)

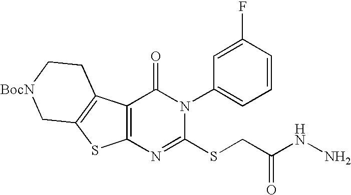

¹H NMR (CDCl₃ 500 MHz) δ (ppm): 7.71 (bs, 1H), 7.54 (td, 1H, J=8 Hz, J′=6 Hz), 7.28 (m, 1H), 7.10 (d, 1H, J=8 Hz), 7.05 (dt, 1H, J=8 Hz, J′=2 Hz), 4.63 (bs, 2H), 3.90 (bs, 2H), 3.78 (d, 1H, J=15 Hz), 3.74 (d, 1H, J=15 Hz), 3.69 (bm, 2H), 3.03 (bs, 2H), 1.49 (s, 9H).

[3-(3-Fluorophenyl)-4-oxo-3,4,5,6,7,8-hexahydro-9-thia-1,3,7-triazafluoren-2-ylsulfanyl]acetic acid hydrazide (986-47)

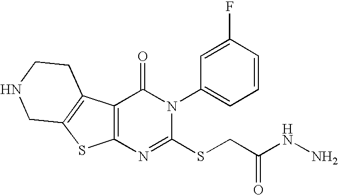

¹H NMR (DMSO-d₆ 500 MHz) δ (ppm): 9.30 (bs, 1H), 7.63 (m, 1H), 7.46 (m, 2H), 7.32 (d, 1H, J=7 Hz), 4.26 (bs, 2H), 3.87 (s, 2H), 3.82 (s, 2H), 2.93 (t, 2H, J=5.7 Hz), 2.76 (m, 2H).

[3-(3-Fluorophenyl)-7-methyl-4-oxo-3,4,5,6,7,8-hexahydro-9-thia-1,3,7-triazafluoren-2-ylsulfanyl]acetic acid hydrazide (986-48)

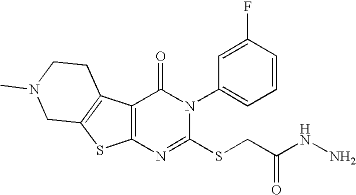

¹H NMR (CDCl₃ 500 MHz) δ (ppm): 7.76 (bs, 1H), 7.53 (td, 1H, J=8 Hz, J′=6 Hz), 7.26 (m, 1H), 7.11 (d, 1H, J=8 Hz), 7.05 (dt, 1H, J=8 Hz, J′=2 Hz), 3.90 (bs, 2H), 3.76 (d, 1H, J=14.5 Hz), 3.73 (d, 1H, J=15 Hz), 3.64 (s, 2H), 3.06 (m, 2H), 2.76 (t, 2H, J=5.7 Hz), 2.51 (s, 3H).

[3-(3-Fluorophenyl)-4-oxo-7-propyl-3,4,5,6,7,8-hexahydro-9-thia-1,3,7-triazafluoren-2-ylsulfanyl]acetic acid hydrazide (986-49)

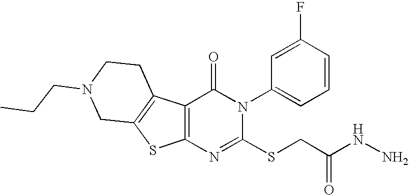

¹H NMR (DMSO-d₆ 500 MHz) δ (ppm): 9.29 (bs, 1H), 7.62 (m, 1H), 7.45 (m, 2H), 7.32 (d, 1H, J=6.5 Hz), 4.26 (bs, 2H), 3.82 (s, 2H), 3.61 (s, 2H), 2.84 (m, 2H), 2.71 (t, 2H, J=6 Hz), 2.45 (t, 2H, J=7.5 Hz), 1.52 (m, 2H), 0.88 (t, 3H, J=7.2 Hz).

### Example 12

### Reaction with Methyl Hydrazine

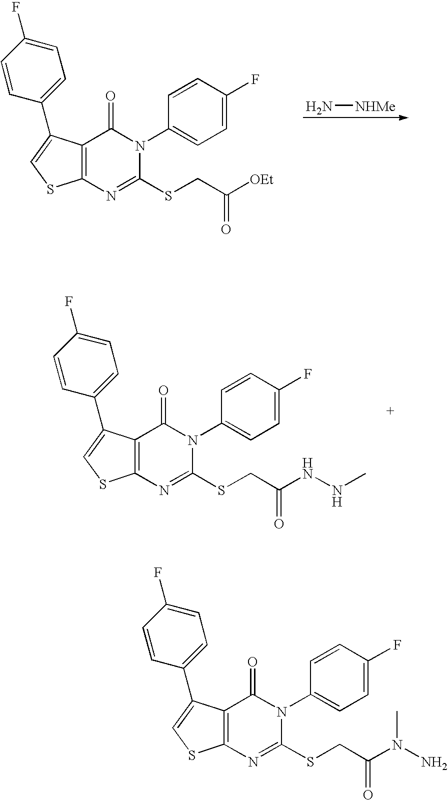

A mixture of [3,5-bis-(4-fluorophenyl)-4-oxo-3,4-dihydrothieno[2,3-d]pyrimidin-2-ylsulfanyl]acetic acid ethyl ester (1 eq.), methyl hydrazine (15 eq.) and ethanol (0.4 M) was heated at 80° C. in a closed tube for 2 h. The solvent was evaporated and the residue purified by silica gel chromatography, eluting with dichloromethane and methanol, to afford [3,5-bis-(4-fluorophenyl)-4-oxo-3,4-dihydrothieno[2,3-d]pyrimidin-2-ylsulfanyl]acetic acid N-methylhydrazide (ldn0058481) and [3,5-bis-(4-fluorophenyl)-4-oxo-3,4-dihydrothieno[2,3-d]pyrimidin-2-ylsulfanyl]acetic acid N'-methylhydrazide (986-51).

[3,5-Bis-(4-fluorophenyl)-4-oxo-3,4-dihydrothieno[2,3-d]pyrimidin-2-ylsulfanyl]acetic acid N-methylhydrazide (986-50)

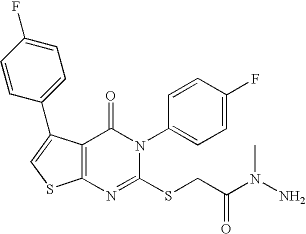

¹H NMR (CDCl₃, 500 MHz) δ (ppm): 7.48 (m, 2H), 7.30 (m, 2H), 7.19 (m, 2H), 7.02 (m, 2H), 6.97 (s, 1H), 4.37 (s, 2H), 3.95 (bs, 2H), 3.22 (s, 3H).

[3,5-Bis-(4-fluorophenyl)-4-oxo-3,4-dihydrothieno[2,3-d]pyrimidin-2-ylsulfanyl]acetic acid N′-methylhydrazide (986-51)

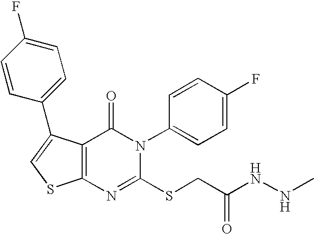

¹H NMR (CDCl₃, 500 MHz) δ (ppm): 7.94 (bs, 1H), 7.48 (m, 2H), 7.28 (m, 2H), 7.21 (m, 2H), 7.04 (m, 3H), 3.75 (s, 2H), 2.65 (s, 3H).

### Example 13

### Reaction with Methylamine

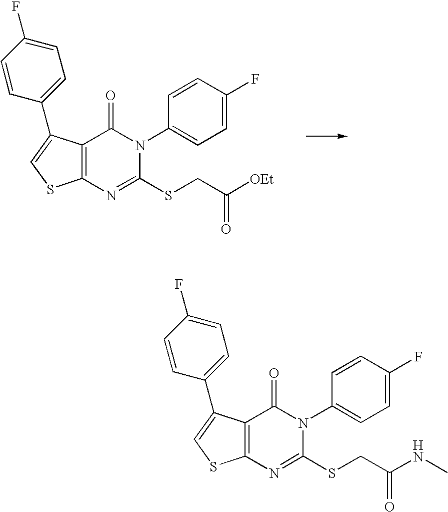

A solution of methylamine in methanol (2 M, 20 eq.) was added to [3,5-15 bis-(4-fluorophenyl)-4-oxo-3,4-dihydrothieno[2,3-d]pyrimidin-2-ylsulfanyl]acetic acid ethyl ester (1 eq.). The mixture was stirred 3 h at room temperature, then poured into 1N HCl and extracted with dichloromethane. The combined organic layers were dried on anhydrous sodium sulfate, filtered and evaporated. The residue was purified by silica gel chromatography, eluting with hexane and ethyl acetate, to afford 2-[3,5-bis-(4-fluorophenyl)-4-oxo-3 ,4-dihydrothieno[2,3 -d]pyrimidin-2-ylsulfanyl]-N-methylacetamide (986-52).

2-[3,5-Bis-(4-fluorophenyl)-4-oxo-3,4-dihydrothieno[2,3-d]pyrimidin-2-ylsulfanyl]-N-methylacetamide (986-52)

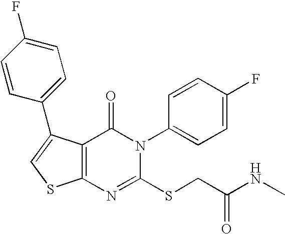

¹H NMR (CDCl₃, 500 MHz) δ (ppm): 7.48 (m, 2H), 7.28 (m, 2H), 7.21 (m, 2H), 7.03 (m, 3H), 6.57 (bs, 1H), 3.79 (s, 2H), 2.85 (d, 3H, J=5 Hz).

### Example 14

### Reaction with Ammonia

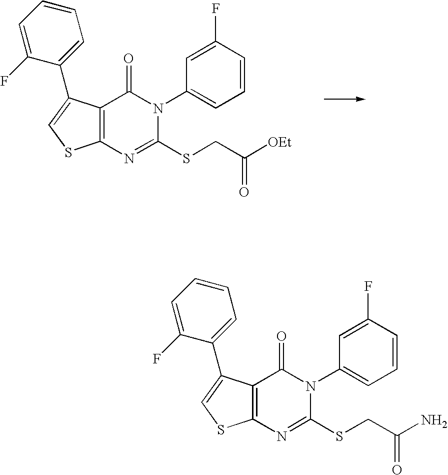

A solution of [3-(3-fluorophenyl)-5-(2-fluorophenyl)-4-oxo-3,4-dihydrothieno[2,3-d]pyrimidin-2-ylsulfanyl]acetic acid ethyl ester (1 eq.) in ethanol (0.2 M) was saturated with ammonia. The mixture was stirred 16 h at room temperature and then evaporated. The residue was purified by silica gel chromatography, eluting with hexane and ethyl acetate, to afford 2-[3-(3-Fluorophenyl)-5-(2-fluorophenyl)-4-oxo-3 ,4-dihydrothieno[2,3-d]pyrimidin-2-ylsulfanyl]acetamide (986-53).

2-[3-(3-Fluorophenyl)-5-(2-fluorophenyl)-4-oxo-3,4-dihydrothieno[2,3-d]pyrimidin-2-ylsulfanyl]acetamide (986-53)

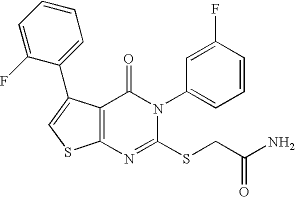

¹H NMR (CDCl₃, 500 MHz) δ (ppm): 7.49 (td, 1H, J=8 Hz, J′=6 Hz), 7.37 (td, 1H, J=8 Hz, J′=2 Hz), 7.31 (m, 1H), 7.22 (td, 1H, J=8 Hz, J′=2 Hz), 7.03-7.16 (m, 5H), 6.56 (bs, 1H), 5.46 (bs, 1H), 3.83 (d, 1H, J=15 Hz), 3.79 (d, 1H, J=15.5 Hz).

### Example 15

### Saponification

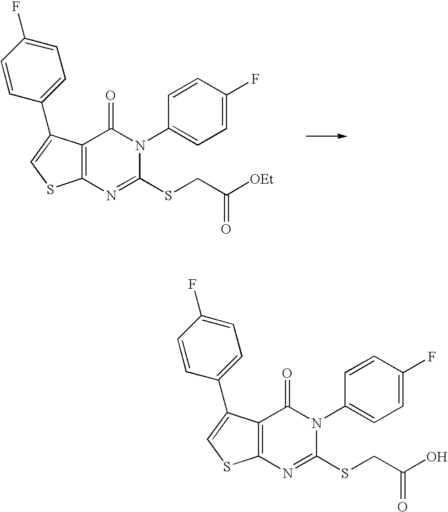

[3,5-Bis-(4-fluorophenyl)-4-oxo-3,4-dihydrothieno[2,3-d]pyrimidin-2-ylsulfanyl]acetic acid ethyl ester (1 eq.) was dissolved in a mixture of THF and methanol (1/1, 0.25 M) and the resulting solution cooled to 0° C. An aqueous solution of sodium hydroxide (10 N, 5 eq.) was added. The reaction mixture was stirred 2 h at 0° C., then poured into 1N HCl and extracted with dichloromethane. The combined organic layers were dried over anhydrous sodium sulfate, filtered and concentrated to afford [3,5-Bis-(4-fluorophenyl)-4-oxo-3,4-dihydrothieno[2,3-d]pyrimidin-2-ylsulfanyl]acetic acid (986-54) as a solid.

[3,5-Bis-(4-fluorophenyl)-4-oxo-3,4-dihydrothieno[2,3-d]pyrimidin-2-ylsulfanyl]acetic acid (986-54)

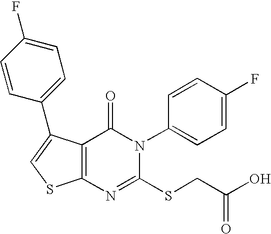

¹H NMR (CDCl₃, 500 MHz) δ (ppm): 7.48 (m, 2H), 7.30 (m, 2H), 7.23 (m, 2H), 7.03 (m, 3H), 3.91 (s, 2H).

### Example 16

### Hydroxamic Acid

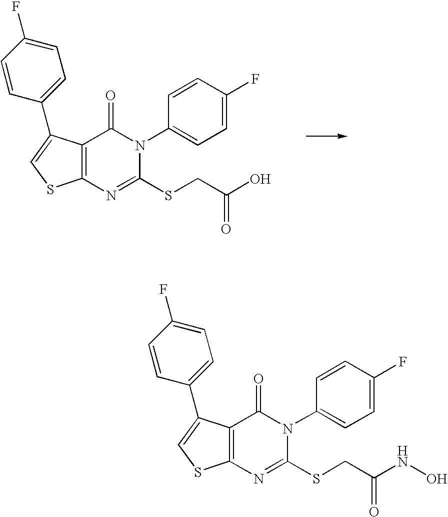

Oxalyl chloride (91 μL, 1.2 eq.) and DMF (1 drop) were added at 0° C. under argon to a suspension of [3,5-bis-(4-fluorophenyl)-4-oxo-3,4-dihydrothieno[2,3-d]pyrimidin-2-ylsulfanyl]acetic acid (373 mg, 1 eq.) in dichloromethane (4.3 mL, 0.2 M). The mixture was stirred for 2 h at room temperature and then added to a mixture of hydroxylamine hydrochloride (301 mg, 5 eq.), Et₃N (608 μL, 5 eq.), THF and water (10/1 ratio, 5 mL). The reaction mixture was stirred 4 h at room temperature and then poured into 1N HCl and extracted sequentially with dichloromethane and AcOEt. The combined organic layers were combined, dried over anhydrous sodium sulfate, filtered and concentrated. The residue was purified by silica gel chromatography, eluting with dichloromethane and methanol (3%), to afford 2-[3,5-bis-(4-fluorophenyl)-4-oxo-3 ,4-dihydrothieno[2,3 -d]pyrimidin-2-ylsulfanyl]-N-hydroxyacetamide.

2-[3,5-Bis-(4-fluorophenyl)-4-oxo-3,4-dihydrothieno[2,3-d]pyrimidin-2-ylsulfanyl]-N-hydroxyacetamide (986-55)

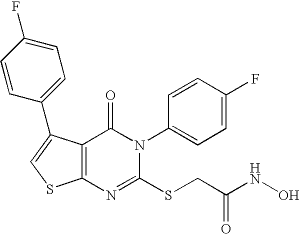

¹H NMR (CDCl₃, 500 MHz) δ (ppm): 9.40 (bs, 1 H), 7.48 (dd, 2H, J=9 Hz, J′=5 Hz), 7.27 (m, 2H), 7.21 (m, 2H). 7.04 (m, 3H), 3.77 (s, 2H).

MS (ESI): M+H, 446.05

### Example 17

### Formation of the α-aminoketone

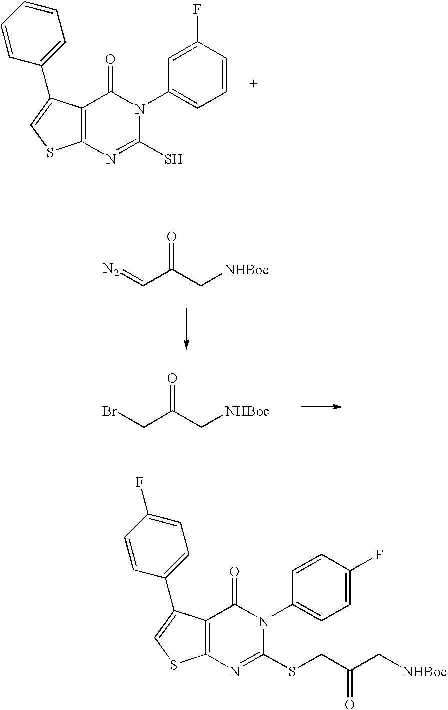

A solution of tert-butyl (3-diazo-2-oxo-propyl)carbamate (80 mg, 2 eq.) in ether (2 mL, 0.2 M) was treated at 0° C. with concentrated HBr (48% in water, 70 μL) and stirred at 0° C. for 30 min. The mixture was then quenched with saturated NaHCO₃, extracted with ether; the combined organic layers were washed with brine, dried over anhydrous Na₂SO₄, filtered and concentrated. The residue was dissolved in DMF (0.4 mL), and to this solution were added 3-(3-fluorophenyl)-2-mercapto-5-phenyl-3H-thieno[2,3-d]pyrimidin-4-one (70 mg, 1 eq.) and K₂CO₃ (41 mg, 1.5 eq.). The reaction mixture was stirred at room temperature for 16 h. The solvent was evaporated and the residue purified by silica gel chromatography, eluting with hexane and ethyl acetate, to afford {3-[3-(3-Fluorophenyl)-4-oxo-5-phenyl-3,4-dihydrothieno[2,3-d]pyrimidin-2-ylsulfanyl]-2-oxo-propyl}carbamic acid tert-butyl ester (114 mg) as an oil.

{3-[3-(3-Fluorophenyl)-4-oxo-5-phenyl-3,4-dihydrothieno[2,3-d]pyrimidin-2-ylsulfanyl]-2-oxo-propyl}carbamic acid tert-butyl ester

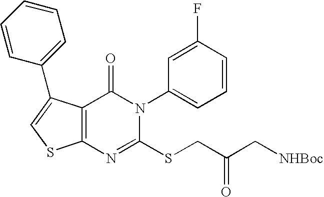

¹H NMR (CDCl₃, 500 MHz) δ (ppm): 7.50 (m, 3H), 7.34 (m, 3H), 7.23 (td, 1H, J=8.2 Hz, J′=2 Hz), 7.13 (d, 1H, J=8 Hz), 7.07 (dt, 1H, J=8.5 Hz, J′=2 Hz), 7.03 (s, 1H), 5.22 (bs, 1H), 4.30 (bd, 2H, J=5.5 Hz), 3.95 (d, 1H, J=16.5 Hz), 3.90 (d, 1H, J=16.5 Hz), 1.46 (s, 9H).

A solution of the {3-[3-(3-fluorophenyl)-4-oxo-5-phenyl-3,4-dihydrothieno[2,3-d]pyrimidin-2-ylsulfanyl]-2-oxo-propyl}carbamic acid tert-butyl ester (78 mg, 1 eq.) in methanol (1.5 mL, 0.1 M) was treated with HCl in ether (2 M, excess). The mixture was stirred 16 h at room temperature, the solvents were evaporated and the residue triturated in ether to afford 2-(3-amino-2-oxo-propylsulfanyl)-3-(3-fluorophenyl)-5-phenyl-3H-thieno[2,3 -d]pyrimidin-4-one hydrochloride as a solid.

2-(3-Amino-2-oxo-propylsulfanyl)-3-(3-fluorophenyl)-5-phenyl-3H-thieno[2,3-d]pyrimidin-4-one hydrochloride (986-56)

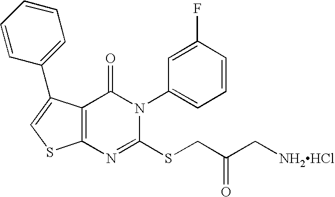

¹H NMR (DMSO-d₆, 500 MHz) δ (ppm): 8.24 (bs, 2H), 7.64 (m, 1H), 7.44-7.52 (m, 5H), 7.35 (m, 4H), 4.21 (s, 2H), 4.15 (m, 2H).

### Example 18

### Synthesis of [3-(3-fluorophenyl)-4-oxo-5-phenyl-3,4-dihydroquinazolin-2-ylsulfanyl]acetic acid hydrazide

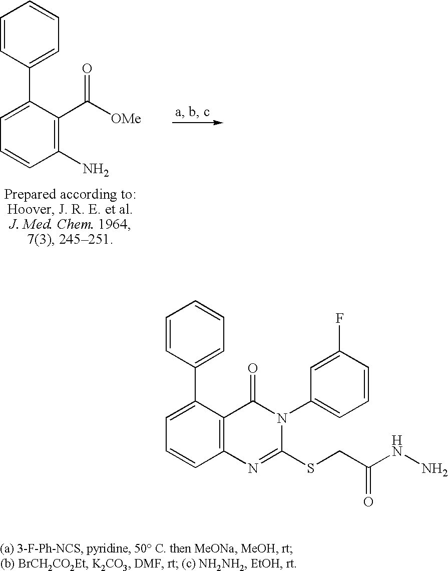

### Example 19

### Structure-activity Relationship Study of Tissue Transglutaminase Inhibitors

This example explored the structure-activity-relationship (SAR) for a class of inhibitors of a specific transglutaminase isopeptide, transglutaminase 2(TGase 2), in an assay that monitored the increase in fluorescence intensity (FI) that accompanies incorporation of the dansylated Lys derivative, α-N-Boc-Lys-CH₂-CH₂-dansyl (KXD), into the protein substrate, N,N-dimethylated-casein (NMC) as described in (Case et al., Biochemistry 2003, 42, 9466-9481). The compounds were based on the following structure (1):

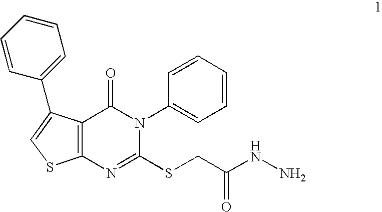

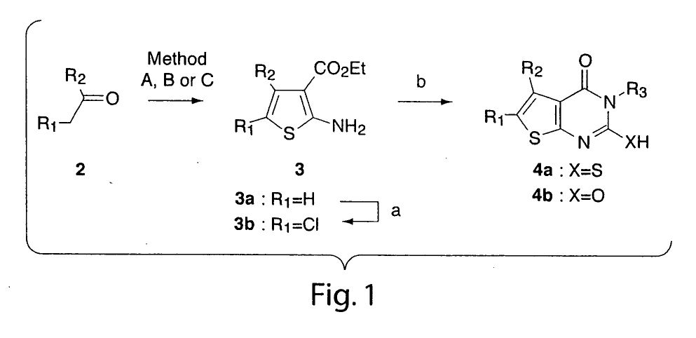

The 2-aminothiophene-3-carboxylates 3 were prepared from aldehydes or ketones 2 using the Gewald reaction (FIG. 1). Depending on the starting material different reaction conditions were used (Method A (as described in Buchstaller et al., Monatsh. Chem. 2001, 132, 279-293): R₁=i-Pr or aryl, R₂=H, Me or i-Pr; Method B, as described in Andersen et al., J Med. Chem. 2002, 45, 4443-4449): for cyclohexanone derivatives and N-Boc-4-piperidone; Method C: R₁=H, R₂=Ph). The 2-chlorothiophene 3b was obtained from 3a by sequential protection of the amine, chlorination of the thiophene ring with sulfuryl chloride (Russell et al., J Med. Chem. 1988, 31, 1786-1793) followed by amine de-protection. Reaction of 3 with alkyl or aryl isothiocyanates (or isocyanates) in basic conditions afforded compounds 4a (or 4b) (Sasaki et al., J Med. Chem. 2003, 46, 113-124).

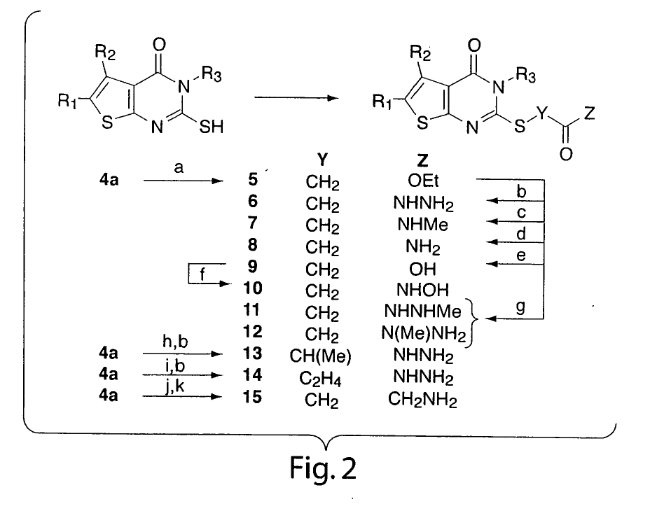

Alkylation of thiol 4a with ethyl bromoacetate followed by aminolysis of the resulting ester 5 with hydrazine led to acylhydrazine 6 (FIG. 2). Compound 5 was also converted into amides 7 and 8, acid 9 and hydroxamic acid 10. Reaction of 5 with methylhydrazine led to a separable mixture of regioisomers 11 and 12. Similarly, reaction of 4a to methyl chloropropionate followed by aminolysis led to the acylhydrazine 13. The homologated analog 14 was accessible by a Michael addition of 4a with methylacrylate followed by aminolysis of the ester with hydrazine. Alternatively, alkylation of 4a with the α-bromoketone BrCH₂C(O)CH₂NHBoc, 16 (prepared in two steps from N-Boc-glycine, (a) Podlech, J.; Seebach, D. Liebig Ann. 1995, 1217-1228; (b) Sengupta, S.; Mondal, S. Tetrahedron 2002, 58, 7983-7986), followed by de-protection of the amine led to the α-aminoketone 15.

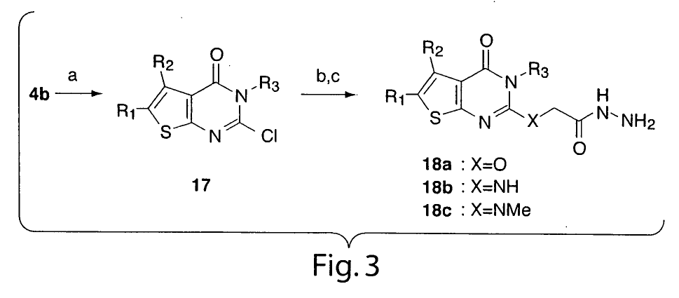

The oxygen and nitrogen analogs 18a-c (FIG. 3) were obtained in three steps from compound 4b. First, 4b was converted into chloride 17 by treatment with POCl₃ under microwave (MW) irradiation. Addition of the requisite nucleophile to 17 followed by aminolysis of the ester gave 18a-c.

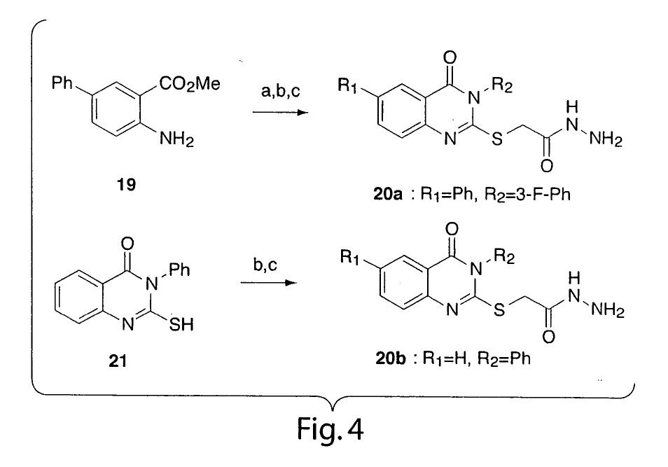

Compounds 20a and 20b, in which the thiophene moiety has been replaced with a benzene, were prepared from 19 and 21 using the same procedures that were employed for the thiophene series (FIG. 4).

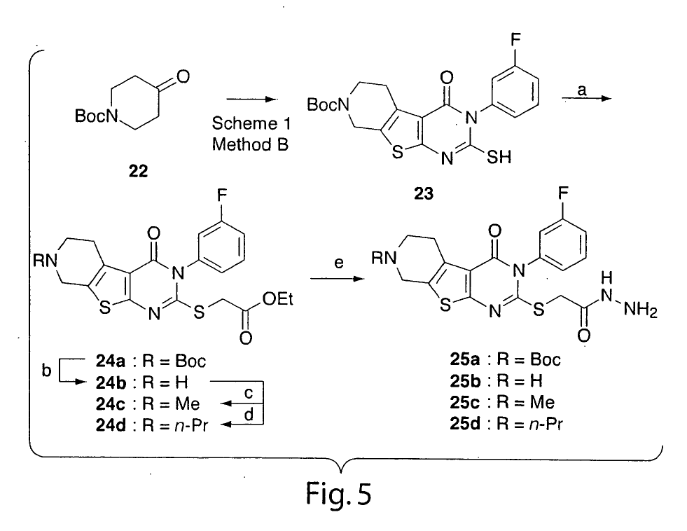

The N-Boc-protected thiophene derivative 23 (FIG. 5), prepared from N-Boc-4-piperidone, 22, utilizing methodology described in Scheme 1/Method B, was alkylated with ethyl bromoacetate to afford 24a. The amine was de-protected to give 24b, which in turn was alkylated to afford 24c and 24d. Each of these derivatives (24a-d) was subsequently converted into acylhydrazines 25a-d.

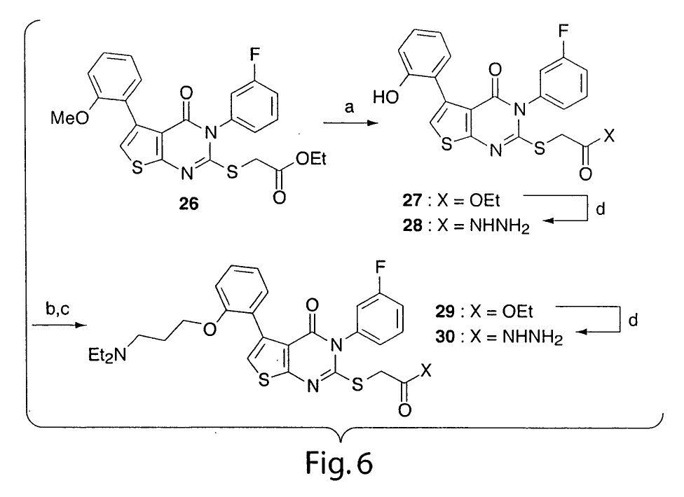

The phenol derivative 27 (FIG. 6) was obtained by first demethylating methyl ether 26. Next, the hydroxyl group was utilized to introduce a diethylaminopropyl side chain in two steps to give 29. Finally, both 27 and 29 were converted into acylhydrazines 28 and 30.

The compounds were evaluated for TGase 2 inhibitory activity utilizing a previously reported assay (Case et al., Biochemistry 2003, 42, 9466-9481). Transpeptidase activity was monitored as the increase in fluorescence intensity (FI) that accompanies incorporation of the dansylated Lys derivative, α-N-Boc-Lys-CH₂-CH₂-dansyl (KXD), into the protein substrate, N,N-dimethylated-casein (NMC). In the standard format (Std) of this assay (Tables 1-4), reactions in the presence of inhibitor were terminated after 60 min and the FI recorded IC₅₀ values were calculated using a four-parameter fit from the dependence of the FI values on inhibitor concentration.

It was noted that some inhibitors exhibited a phenomenon known as “slow-binding inhibition.” The inhibitor binds to the enzyme on a time scale of minutes rather than the time scale of classical inhibitors (milliseconds) (Morrison et al., Adv. Enzym. 1988, 61, 201-301). For these compounds, this standard format provides less accurate IC₅₀ values. Therefore, a method was used in which full progress curves (FPC) were recorded for the TGase-catalyzed reaction of KXD with NMC in the presence of inhibitor (Table 1-4). Reactions were initiated by enzyme addition to a solution of substrate and inhibitor. Reaction progress curves were characterized by an initial rapid velocity, frequently equal to control velocity, vc, in the absence of inhibitor (I), followed by a first-order decrease in velocity to the final steady-state velocity, vₛₛ, that reflects the full potency of the compound. From such progress curves, IC₅₀ values were calculated using the following equation: IC₅₀=[I]/{(v_{c}/vₛₛ)−1}. Progress curves recorded at several inhibitor concentrations were used to determine FPC IC₅₀ values (Table 1-4). Standard errors were typical <15% of these values.

The replacement of the thiophene with a benzene ring, a known bioisoster, was briefly studied (Table 1). Compound 986-03 exhibited decreased activity compared to the original inhibitor 1. Activity was partly restored by adding a phenyl substituent at R₁ (986-04).

TABLE 1
Quinazolin-4-one derivatives prepared for structure - activity relationship
studies and IC₅₀ values for TGase 2 inhibition.

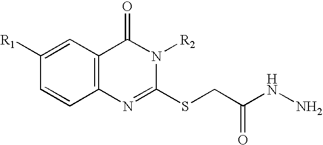

| Cmpd | R₁ | R₂ | IC₅₀ (μM, Std) | IC₅₀ (μM, FPC) |
|---|---|---|---|---|
| 986-03 | H | Ph | 18 | — |
| 986-04 | Ph | 3-F—Ph | — | 1.5 |

A second series of analogs was generated, with the purpose of replacing the thioether and acylhydrazide moieties (X and R₄, Table 2). Replacement of the sulfur atom with an oxygen (986-30) or a nitrogen (986-05) resulted in decreased activity, possibly due to the shorter C—X bond length. Homologation of the side chain R₄ (986-22) also led to diminished activity. All modifications of the acylhydrazide met with little success: the α- and β-methylhydrazides, acid, ester, amides and hydroxamic acid analogs showed lower inhibition at 20 μM. Only the α-aminoketone exhibited some activity, albeit weaker than the acylhydrazide.

TABLE 2
Thieno[2,3-d]pyrimidin-4-one derivatives prepared for structure -
activity relationship studies and IC₅₀ values for TGase 2 inhibition.

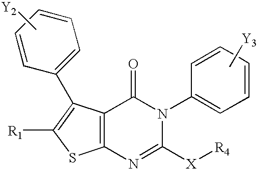

| | | | | | | IC₅₀ | IC₅₀ |
|---|---|---|---|---|---|---|---|
| Cmpd | R₁ | Y₂ | Y₃ | X | R₄ | (μM, Std) | (μM, FPC) |
| 986-01 | H | H | H | S | CH₂C(O)NHNH₂ | 0.8 | 0.25 |
| 986-30 | H | H | H | O | CH₂C(O)NHNH₂ | 4.5 | — |
| 986-05 | H | H | H | NH | CH₂C(O)NHNH₂ | — | 3.7 |
| 986-22 | Me | H | H | S | CH₂CH₂C(O)NHNH₂ | 1.3 | — |
| 986-12 | H | H | H | S | CH(Me)C(O)NHNH₂ | >10 | — |
| 986-06 | H | H | H | NMe | CH₂C(O)NHNH₂ | >20 | — |
| 986-50 | H | 4-F | 4-F | S | CH₂C(O)NMeNH₂ | >20 | — |
| 986-51 | H | 4-F | 4-F | S | CH₂C(O)NHNHMe | >20 | — |
| 986-54 | H | 4-F | 4-F | S | CH₂CO₂H | >20 | — |
| 986-58 | H | H | 4-F | S | CH₂CO₂Et | >20 | — |
| 986-52 | H | 4-F | 4-F | S | CH₂C(O)NHMe | >20 | — |
| 986-53 | H | 2-F | 3-F | S | CH₂C(O)NH₂ | >10 | — |
| 986-55 | H | 4-F | 4-F | S | CH₂C(O)NHOH | >20 | — |
| 986-56^{a} | H | H | 3-F | S | CH₂C(O)CH₂NH₂ | 5.3 | — |

^{a}HCl salt

Next, a SAR study of the R₁, R₂ and R₃ substituents was undertaken (Table 3). Replacement of the phenyl at R₃ with a methyl, cyclohexyl, benzyl or 3-pyridyl decreased activity. Methoxy or chloro substitution on the R₃ phenyl decreased activity, but introduction of a fluoro group led to equal or slightly better activities. Replacing the phenyl substituent at R₂ with a 2-fluorophenyl or a 2- or 3-methoxyphenyl led to a two-fold increase in activity. However, an attempt to increase solubility by introducing a basic amine into the ether side-chain (986-35) resulted in decreased inhibitory activity. The R₂ phenyl group could also be replaced with an isopropyl group (986-36) with no loss in activity. Finally, substitution at R₁ was studied. Introduction of a methyl (986-13, mp 227-228 ° C.; ¹H NMR (CDCl₃, 400 MHz): δ 7.90 (bs, 1H), 7.48 (m, 3H), 7.36 (t, 2H, J=7.3 Hz), 7.30 (m, 3H), 7.24 (m, 2H), 3.92 (bs, 2H), 3.76 (s, 2H), 2.37 (s, 3H); ¹³C NMR, (CDCl₃, 100 MHz) δ (ppm): 169.2, 161.3, 157.5, 157.1, 135.2, 135.1, 134.4, 132.1, 130.4, 130.3, 130.0, 129.3, 127.9, 127.6, 119.6, 34.0, 14.1) or chloro (986-39) substituent increased activity, whereas an isopropyl (986-41) was detrimental. Interestingly, transposing the phenyl group from R₂ to the R₁ position (986-29) resulted in loss of inhibitory activity.

TABLE 3
Thieno[2,3-d]pyrimidin-4-one derivatives prepared for structure -
activity relationship studies and IC₅₀ values for TGase 2 inhibition.

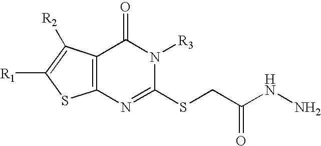

| Cmpd | R₁ | R₂ | R₃ | IC₅₀ (μM, Std) | IC₅₀ (μM, FPC) |
|---|---|---|---|---|---|
| 986-01 | H | Ph | Ph | 0.8 | 0.25 |
| 986-09 | H | 4-F—Ph | 4-F—Ph | 1.5 | 0.67 |
| 986-10 | H | Ph | Me | 8.4 | — |
| 986-14 | H | Ph | CH₂Ph | 2.3 | — |
| 986-16 | H | Ph | 3-Py | 1.2 | 0.48 |
| 986-17 | H | Ph | Cy | 2.6 | — |
| 986-20 | H | Ph | 2-OMe—Ph | 2.0 | — |
| 986-21 | H | Ph | 3-OMe—Ph | 0.82 | 0.47 |
| 986-15 | H | Ph | 4-OMe—Ph | 2.1 | — |
| 986-23 | H | Ph | 2-Cl—Ph | 1.8 | — |
| 986-24 | H | Ph | 3-Cl—Ph | 1.5 | — |
| 986-25 | H | Ph | 4-Cl—Ph | 1.8 | — |
| 986-18 | H | Ph | 2-F—Ph | 0.50 | 0.16 |
| 986-19 | H | Ph | 3-F—Ph | 0.25 | 0.18 |
| 986-07 | H | Ph | 4-F—Ph | 1.3 | 0.32 |
| 986-26 | H | Ph | 2,5-di-F—Ph | 0.59 | 0.23 |
| 986-27 | H | Ph | 3,5-di-F—Ph | 0.48 | 0.22 |
| 986-28 | H | Ph | 2,6-di-F—Ph | 20 | — |
| 986-11 | H | Me | 4-F—Ph | 6.3 | — |
| 986-36 | H | iPr | 3-F—Ph | — | 0.23 |
| 986-33 | H | 4-OMe—Ph | 3-F—Ph | — | 0.28 |
| 986-32 | H | 3-OMe—Ph | 3-F—Ph | 0.21 | 0.14 |
| 986-31 | H | 2-OMe—Ph | 3-F—Ph | — | 0.14 |
| 986-34 | H | 2-OH—Ph | 3-F—Ph | 1.4 | — |
| 986-35 | H | 2-(OC₃H₆NEt₂)—Ph | 3-F—Ph | 4.8 | — |
| 986-08 | H | 4-F—Ph | Ph | 0.80 | 0.29 |
| 986-38 | H | 3-F—Ph | 3-F—Ph | — | 0.29 |
| 986-37 | H | 2-F—Ph | 3-F—Ph | — | 0.14 |
| 986-39 | Cl | 2-F—Ph | 3-F—Ph | — | 0.13 |
| 986-13 | Me | Ph | Ph | 1.5 | 0.16 |
| 986-40 | Me | 2-F—Ph | 3-F—Ph | — | 0.15 |
| 986-41 | iPr | 2-F—Ph | 3-F—Ph | — | 0.90 |
| 986-29 | Ph | H | Ph | >20 | — |

Analogs bearing a fused cyclohexyl or a piperidinyl moiety on the thiophene were also prepared (Table 4). Whereas the piperidinyl derivatives were generally less potent inhibitors, the cyclohexyl derivatives exhibited activities more reminiscent of 1. Introducing substituents (i.e. R=Me or Ph) on the fuse cyclohexyl ring modestly increased activity.

TABLE 4
Thieno[2,3-d]pyrimidin-4-one derivatives prepared for structure -
activity relationship studies and IC₅₀ values for TGase 2 inhibition.

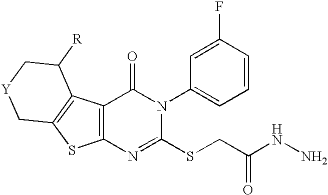

| Cmpd | Y | R | IC₅₀ (μM, Std) | IC₅₀ (μM, FPC) |
|---|---|---|---|---|
| 986-42 | CH₂ | H | — | 0.45 |
| 986-43 | CH₂ | Me | — | 0.20 |
| 986-44 | CH₂ | Ph | 0.93 | 0.17 |
| 986-45 | CH₂ | CH₂Ph | 2.3 | — |
| 986-46 | NBoc | H | — | 1.1 |
| 986-47 | NH | H | — | 7.1 |
| 986-48 | NMe | H | — | 0.53 |
| 986-49 | N—n-Pr | H | 6.0 | — |

A SAR study for TGase 2 inhibition by thieno[2,3-d]pyrimidin-4-one acylhydrazides revealed several interesting findings. First, the acylhydrazide side-chain increased inhibitory activity and modifications of the acylhydrazide side-chain decreased inhibitory activity. Also, the thiophene ring increased inhibitory activity. The other substituents were tolerant to some changes resulting in compounds (986-18, 986-19, 986-32, 986-31, 986-37, 986-39, 986-13, and 986-40) with FPC IC₅₀ values <0.16 μM.

This example illustrates that SAR studies can be carried out to derive specific compounds that have inhibitory activity towards particular transglutaminase isoforms, in this case Tgase 2. It will be appreciated that the results of this example measured the inhibitory activity of this class of compounds in an in vitro assay based on one transglutaminase activity. For the therapeutic methods of the invention, it may be desirable to use a transglutaminase inhibitor that inhibits only certain transglutaminase activities or certain transglutaminase isoforms and/or inhibits them only to a certain degree. Although some compounds have lower inhibitory activity in this in vitro assay, that does not signify that they would have lower threapeutic value.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims.

What is claimed is:

1. A compound of the formula: 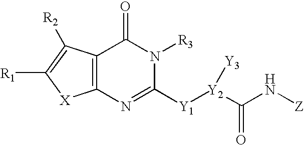
a stereoisomeric form thereof, or a pharmaceutically acceptable acid or base addition salt thereof;
wherein
R₁ is selected from the group consisting of: H, halogen, alkyl, substituted alkyl, aryl and substituted aryl;
R₂ is selected from the group consisting of: H, alkyl, substituted alkyl, aryl and substituted aryl;
R₃ is selected from the group consisting of: alkyl, substituted alkyl, aryl, substituted aryl, pyridine and substituted pyridine;
X is selected from the group consisting of: S, O and NH;
Y₁ is selected from the group consisting of: S, CH₂, NH, O and N-alkyl;
Y₂ is selected from the group consisting of: CH, alkyl and substituted alkyl;
Y₃ is selected from the group consisting of: H and CH₃;
Z is selected from the group consisting of: OH and NH₂;
with the provision that
when X is S, Y₁ is S, Y₂ is CH, Y₃ is H, Z is NH₂, R₁ is Me, and R₂ is Me,
R₃ can not be Ph or a propylene group (CH₂═CH—CH₂—); and
when X is S, Y₁ is S, Y₂ is CH, Y₃ is H, Z is NH₂, R₁ is H, and R₂ is Ph, R₃ can not be Ph.

2. The compound of claim 1, a stereoisomeric form thereof, or a pharmaceutically acceptable acid or base addition salt thereof, wherein
R₁ is selected from the group consisting of: H, Me and Cl;
R₂ is selected from the group consisting of: phenyl and substituted phenyl;
R₃ is selected from the group consisting of: phenyl and substituted phenyl;
X is S;
Y₁ is S;
Y₂ is CH;
Y₃ is H; and
Z is NH₂.

3. A pharmaceutical preparation comprising a compound of claim 1 and a pharmaceutically acceptable carrier.

4-5. (canceled)

6. A method of treating a transglutaminase-associated disorder comprising administering to a subject having the transglutaminase-associated disorder a compound of claim 1
wherein the compound is administered in an amount effective to treat the transglutaminase-associated disorder.

7. The method of claim 6, wherein the transglutaminase-associated disorder is a neurodegenerative disorder chosen from the list consisting of: Parkinson's disease, Alzheimer's disease, and progressive supranuclear palsy.

8. (canceled)

9. The method of claim 6, wherein the transglutaminase-associated disorder is a CAG-expansion disorder chosen from the list consisting of: spinocerebellar ataxia type 1, spinocerebellar ataxia type 2, Machado-Joseph disorder, spinocerebellar ataxia type 6, spinocerebellar ataxia type 7, spinocerebellar ataxia type 12, spinocerebellar ataxia type 17, spinobulbar muscular atrophy, dentatorubral-pallidoluysian atrophy and Huntington's disease.

10. (canceled)

11. The method of claim 6, wherein the transglutaminase-associated disorder is an autoimmune disorder chosen from the list consisting of: hepatitis, hemolytic anemia, myasthenia, subepidermal blisters, multiple sclerosis, lupus, necrobiosis lipoidica, myasthenia gravis, bullous pemphigoid, Goodpasture disease, rheumatoid arthritis, amyloid lateral sclerosis, inclusion body myositis and celiac spru.

12-53. (canceled)

54. A compound of the formula: 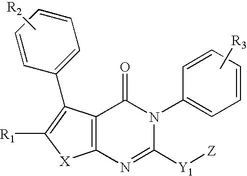
a stereoisomeric form thereof, or a pharmaceutically acceptable acid or base addition salt thereof;
wherein
R₁ is selected from the group consisting of: H, halogen and Me;
R₂ is selected from the group consisting of: H, 4-F, and 2-F;
R₃ is selected from the group consisting of: H, 4-F, and 3-F;
X is selected from the group consisting of: S, O and NH;
Y₁ is selected from the group consisting of: S, O, NH and NMe; and
Z is selected from the group consisting of: CH₂C(O)NHNH₂, CH₂CH₂C(O)NHNH₂, CH(Me)C(O)NHNH₂, CH₂C(O)NMeNH₂, CH₂C(O)NHNHMe, CH₂CO₂H, CH₂CO₂Et, CH₂C(O)NHMe, CH₂C(O)NH₂, CH₂C(O)NHOH, and CH₂C(O)CH₂NH₂.

55. The compound of claim 54, wherein R₁ is Cl or F.

56. A pharmaceutical preparation comprising a compound of claim 54 and a pharmaceutically acceptable carrier.

57-58. (canceled)

59. A method of treating a transglutaminase-associated disorder comprising administering to a subject having the transglutaminase-associated disorder a compound of claim 54
wherein the compound is administered in an amount effective to treat the transglutaminase-associated disorder.

60. (canceled)

61. The method of claim 59, wherein the transglutaminase-associated disorder is a neurodegenerative disorder chosen from the list consisting of: Parkinson's disease, Alzheimer's disease, and progressive supranuclear palsy.

62. (canceled)

63. The method of claim 59, wherein the transglutaminase-associated disorder is a CAG-expansion disorder chosen from the list consisting of: spinocerebellar ataxia type 1, spinocerebellar ataxia type 2, Machado-Joseph disorder, spinocerebellar ataxia type 6, spinocerebellar ataxia type 7, spinocerebellar ataxia type 12, spinocerebellar ataxia type 17, spinobulbar muscular atrophy, dentatorubral-pallidoluysian atrophy and Huntington's disease.

64. (canceled)

65. The method of claim 59, wherein the transglutaminase-associated disorder is an autoimmune disorder chosen from the list consisting of: hepatitis, hemolytic anemia, myasthenia, subepidermal blisters, multiple sclerosis, lupus, necrobiosis lipoidica, myasthenia gravis, bullous pemphigoid, Goodpasture disease, rheumatoid arthritis, amyloid lateral sclerosis, inclusion body myositis and celiac spru.

66-87. (canceled)

88. A compound of the formula: 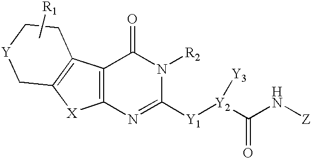
or a stereoisomeric form thereof, a pharmaceutically acceptable acid or base addition salt thereof,
wherein
Y is selected from the group consisting of: CH₂, N-Boc, NH, NMe, N-alkyl; and
R₁ is selected from the group consisting of: H, Me, Ph, alkyl, arylalkyl, t-butyl, and CH₂Ph;
R₂ is selected from the group consisting of: alkyl, substituted alkyl, aryl, substituted aryl; pyridine, and substituted pyridine;
X is selected from the group consisting of: S, O and NH;
Y₁ is selected from the group consisting of: S, CH₂, O, NH, N-alkyl;
Y₂ is selected from the group consisting of: CH, alkyl and substituted alkyl;
Y₃ is selected from the group consisting of: H and CH₃; and
Z is selected from the group consisting of: OH and NH₂.

89. A pharmaceutical preparation comprising a compound of claim 88 and a pharmaceutically acceptable carrier.

90-91. (canceled)

92. A method of treating a transglutaminase-associated disorder comprising administering to a subject having the transglutaminase-associated disorder a compound of claim 88
wherein the compound is administered in an amount effective to treat the transglutaminase-associated disorder.

93. The method of claim 92, wherein the transglutaminase-associated disorder is a neurodegenerative disorder chosen from the list consisting of: Parkinson's disease, Alzheimer's disease, and progressive supranuclear palsy.

94. (canceled)

95. The method of claim 92, wherein the transglutaminase-associated disorder is a CAG-expansion disorder chosen from the list consisting of: spinocerebellar ataxia type 1, spinocerebellar ataxia type 2, Machado-Joseph disorder, spinocerebellar ataxia type 6, spinocerebellar ataxia type 7, spinocerebellar ataxia type 12, spinocerebellar ataxia type 17, spinobulbar muscular atrophy, dentatorubral-pallidoluysian atrophy and Huntington's disease.

96. (canceled)

97. The method of claim 92, wherein the transglutaminase-associated disorder is an autoimmune disorder chosen from the list consisting of: hepatitis, hemolytic anemia, myasthenia, subepidermal blisters, multiple sclerosis, lupus, necrobiosis lipoidica, myasthenia gravis, bullous pemphigoid, Goodpasture disease, rheumatoid arthritis, amyloid lateral sclerosis, inclusion body myositis and celiac spru.

98-103. (canceled)


SEQUENCE LISTING (15 sequences)

SEQ ID NO: 1 - protein, 4 aa, Artificial
  Synthetic peptide
IETD

SEQ ID NO: 2 - protein, 4 aa, Artificial
  Synthetic peptide
YVAD

SEQ ID NO: 3 - protein, 4 aa, Artificial
  Synthetic peptide
DEVD

SEQ ID NO: 4 - protein, 4 aa, Artificial
  Synthetic peptide
LEHD

SEQ ID NO: 5 - protein, 6 aa, Artificial
  Synthetic peptide
RKLMEI

SEQ ID NO: 6 - protein, 8 aa, Artificial
  Synthetic peptide
GTLAKKLT

SEQ ID NO: 7 - protein, 9 aa, Artificial
  Synthetic peptide
SHLRKVFDK

SEQ ID NO: 8 - protein, 9 aa, Artificial
  Synthetic peptide
HDMNKVLDL

SEQ ID NO: 9 - protein, 9 aa, Artificial
  Synthetic peptide
MQMKKVLDS

SEQ ID NO: 10 - protein, 4 aa, Artificial
  Synthetic peptide
KVLD

SEQ ID NO: 11 - protein, 8 aa, Artificial
  Synthetic peptide
KVLDPVKG

SEQ ID NO: 12 - protein, 8 aa, Artificial
  Synthetic peptide
KVLDGQDP

SEQ ID NO: 13 - protein, 4 aa, Artificial
  Synthetic peptide
PVKG

SEQ ID NO: 14 - protein, 5 aa, Artificial
  Synthetic peptide
DPVKG

SEQ ID NO: 15 - protein, 4 aa, Artificial
  Synthetic peptide
GQDP